SEQUENCE LISTING (106 sequences)

SEQ ID NO: 1 - protein, 25 aa, Artificial Sequence
  source: /note=“Description of Artificial Sequence Synthetic peptide”
MTEYKLVVVGACGVGKSALTIQLIQ

SEQ ID NO: 2 - protein, 25 aa, Artificial Sequence
  source: /note=“Description of Artificial Sequence Synthetic peptide”
MTEYKLVVVGAAGVGKSALTIQLIQ

SEQ ID NO: 3 - protein, 25 aa, Artificial Sequence
  source: /note=“Description of Artificial Sequence Synthetic peptide”
MTEYKLVVVGADGVGKSALTIQLIQ

SEQ ID NO: 4 - protein, 25 aa, Artificial Sequence
  source: /note=“Description of Artificial Sequence Synthetic peptide”
MTEYKLVVVGARGVGKSALTIQLIQ

SEQ ID NO: 5 - protein, 25 aa, Artificial Sequence
  source: /note=“Description of Artificial Sequence Synthetic peptide”
MTEYKLVVVGASGVGKSALTIQLIQ

SEQ ID NO: 6 - protein, 25 aa, Artificial Sequence
  source: /note=“Description of Artificial Sequence Synthetic peptide”
MTEYKLVVVGAVGVGKSALTIQLIQ

SEQ ID NO: 7 - protein, 25 aa, Artificial Sequence
  source: /note=“Description of Artificial Sequence Synthetic peptide”
MTEYKLVVVGAGCVGKSALTIQLIQ

SEQ ID NO: 8 - protein, 25 aa, Artificial Sequence
  source: /note=“Description of Artificial Sequence Synthetic peptide”
MTEYKLVVVGAGAVGKSALTIQLIQ

SEQ ID NO: 9 - protein, 25 aa, Artificial Sequence
  source: /note=“Description of Artificial Sequence Synthetic peptide”
MTEYKLVVVGAGDVGKSALTIQLIQ

SEQ ID NO: 10 - protein, 25 aa, Artificial Sequence
  source: /note=“Description of Artificial Sequence Synthetic peptide”
MTEYKLVVVGAGRVGKSALTIQLIQ

SEQ ID NO: 11 - protein, 25 aa, Artificial Sequence
  source: /note=“Description of Artificial Sequence Synthetic peptide”
MTEYKLVVVGAGSVGKSALTIQLIQ

SEQ ID NO: 12 - protein, 25 aa, Artificial Sequence
  source: /note=“Description of Artificial Sequence Synthetic peptide”
MTEYKLVVVGAGVVGKSALTIQLIQ

SEQ ID NO: 13 - protein, 25 aa, Artificial Sequence
  source: /note=“Description of Artificial Sequence Synthetic peptide”
ETCLLDILDTAGEEEYSAMRDQYMR

SEQ ID NO: 14 - protein, 25 aa, Artificial Sequence
  source: /note=“Description of Artificial Sequence Synthetic peptide”
ETCLLDILDTAGHEEYSAMRDQYMR

SEQ ID NO: 15 - protein, 25 aa, Artificial Sequence
  source: /note=“Description of Artificial Sequence Synthetic peptide”
ETCLLDILDTAGKEEYSAMRDQYMR

SEQ ID NO: 16 - protein, 25 aa, Artificial Sequence
  source: /note=“Description of Artificial Sequence Synthetic peptide”
ETCLLDILDTAGLEEYSAMRDQYMR

SEQ ID NO: 17 - protein, 25 aa, Artificial Sequence
  source: /note=“Description of Artificial Sequence Synthetic peptide”
ETCLLDILDTAGPEEYSAMRDQYMR

SEQ ID NO: 18 - protein, 25 aa, Artificial Sequence
  source: /note=“Description of Artificial Sequence Synthetic peptide”
ETCLLDILDTAGREEYSAMRDQYMR

SEQ ID NO: 19 - protein, 30 aa, Artificial Sequence
  source: /note=“Description of Artificial Sequence Synthetic polypeptide”
QDLARSYGIPFIETSTKTRQRVEDAFYTLV

SEQ ID NO: 20 - protein, 30 aa, Artificial Sequence
  source: /note=“Description of Artificial Sequence Synthetic polypeptide”
QDLARSYGIPFIETSVKTRQRVEDAFYTLV

SEQ ID NO: 21 - protein, 30 aa, Artificial Sequence
  source: /note=“Description of Artificial Sequence Synthetic polypeptide”
QDLARSYGIPFIETSPKTRQRVEDAFYTLV

SEQ ID NO: 22 - protein, 25 aa, Artificial Sequence
  source: /note=“Description of Artificial Sequence Synthetic peptide”
GETCLLDILDTADQEEYSAMRDQYM

SEQ ID NO: 23 - protein, 25 aa, Artificial Sequence
  source: /note=“Description of Artificial Sequence Synthetic peptide”
GETCLLDILDTAVQEEYSAMRDQYM

SEQ ID NO: 24 - protein, 25 aa, Artificial Sequence
  source: /note=“Description of Artificial Sequence Synthetic peptide”
GETCLLDILDTARQEEYSAMRDQYM

SEQ ID NO: 25 - protein, 25 aa, Artificial Sequence
  source: /note=“Description of Artificial Sequence Synthetic peptide”
DSEDVPMVLVGNNCDLPSRTVDTKQ

SEQ ID NO: 26 - protein, 25 aa, Artificial Sequence
  source: /note=“Description of Artificial Sequence Synthetic peptide”
DSEDVPMVLVGNRCDLPSRTVDTKQ

SEQ ID NO: 27 - protein, 25 aa, Artificial Sequence
  source: /note=“Description of Artificial Sequence Synthetic peptide”
DGETCLLDILDTTGQEEYSAMRDQY

SEQ ID NO: 28 - protein, 25 aa, Artificial Sequence
  source: /note=“Description of Artificial Sequence Synthetic peptide”
DGETCLLDILDTGGQEEYSAMRDQY

SEQ ID NO: 29 - protein, 25 aa, Artificial Sequence
  source: /note=“Description of Artificial Sequence Synthetic peptide”
DGETCLLDILDTEGQEEYSAMRDQY

SEQ ID NO: 30 - protein, 25 aa, Artificial Sequence
  source: /note=“Description of Artificial Sequence Synthetic peptide”
DGETCLLDILDTSGQEEYSAMRDQY

SEQ ID NO: 31 - protein, 25 aa, Artificial Sequence
  source: /note=“Description of Artificial Sequence Synthetic peptide”
VVVGAGGVGKSAFTIQLIQNHFVDE

SEQ ID NO: 32 - protein, 25 aa, Artificial Sequence
  source: /note=“Description of Artificial Sequence Synthetic peptide”
ETCLLDILDTAGREEYSAMRDQYMR

SEQ ID NO: 33 - protein, 25 aa, Artificial Sequence
  source: /note=“Description of Artificial Sequence Synthetic peptide”
ETCLLDILDTAGKEEYSAMRDQYMR

SEQ ID NO: 34 - protein, 25 aa, Artificial Sequence
  source: /note=“Description of Artificial Sequence Synthetic peptide”
ETCLLDILDTAGLEEYSAMRDQYMR

SEQ ID NO: 35 - protein, 25 aa, Artificial Sequence
  source: /note=“Description of Artificial Sequence Synthetic peptide”
ETCLLDILDTAGHEEYSAMRDQYMR

SEQ ID NO: 36 - protein, 25 aa, Artificial Sequence
  source: /note=“Description of Artificial Sequence Synthetic peptide”
ETCLLDILDTAGPEEYSAMRDQYMR

SEQ ID NO: 37 - protein, 25 aa, Artificial Sequence
  source: /note=“Description of Artificial Sequence Synthetic peptide”
ETCLLDILDTAGEEEYSAMRDQYMR

SEQ ID NO: 38 - protein, 25 aa, Artificial Sequence
  source: /note=“Description of Artificial Sequence Synthetic peptide”
MTEYKLVVVGADGVGKSALTIQLIQ

SEQ ID NO: 39 - protein, 25 aa, Artificial Sequence
  source: /note=“Description of Artificial Sequence Synthetic peptide”
MTEYKLVVVGAAGVGKSALTIQLIQ

SEQ ID NO: 40 - protein, 25 aa, Artificial Sequence
  source: /note=“Description of Artificial Sequence Synthetic peptide”
MTEYKLVVVGACGVGKSALTIQLIQ

SEQ ID NO: 41 - protein, 25 aa, Artificial Sequence
  source: /note=“Description of Artificial Sequence Synthetic peptide”
MTEYKLVVVGARGVGKSALTIQLIQ

SEQ ID NO: 42 - protein, 25 aa, Artificial Sequence
  source: /note=“Description of Artificial Sequence Synthetic peptide”
MTEYKLVVVGASGVGKSALTIQLIQ

SEQ ID NO: 43 - protein, 25 aa, Artificial Sequence
  source: /note=“Description of Artificial Sequence Synthetic peptide”
MTEYKLVVVGAVGVGKSALTIQLIQ

SEQ ID NO: 44 - protein, 25 aa, Artificial Sequence
  source: /note=“Description of Artificial Sequence Synthetic peptide”
MTEYKLVVVGAGCVGKSALTIQLIQ

SEQ ID NO: 45 - protein, 25 aa, Artificial Sequence
  source: /note=“Description of Artificial Sequence Synthetic peptide”
MTEYKLVVVGAGAVGKSALTIQLIQ

SEQ ID NO: 46 - protein, 25 aa, Artificial Sequence
  source: /note=“Description of Artificial Sequence Synthetic peptide”
MTEYKLVVVGAGDVGKSALTIQLIQ

SEQ ID NO: 47 - protein, 25 aa, Artificial Sequence
  source: /note=“Description of Artificial Sequence Synthetic peptide”
MTEYKLVVVGAGRVGKSALTIQLIQ

SEQ ID NO: 48 - protein, 25 aa, Artificial Sequence
  source: /note=“Description of Artificial Sequence Synthetic peptide”
MTEYKLVVVGAGSVGKSALTIQLIQ

SEQ ID NO: 49 - protein, 25 aa, Artificial Sequence
  source: /note=“Description of Artificial Sequence Synthetic peptide”
MTEYKLVVVGAGVVGKSALTIQLIQ

SEQ ID NO: 50 - protein, 25 aa, Artificial Sequence
  source: /note=“Description of Artificial Sequence Synthetic peptide”
GETCLLDILDTAEQEEYSAMRDQYM

SEQ ID NO: 51 - protein, 25 aa, Artificial Sequence
  source: /note=“Description of Artificial Sequence Synthetic peptide”
GETCLLDILDTARQEEYSAMRDQYM

SEQ ID NO: 52 - protein, 25 aa, Artificial Sequence
  source: /note=“Description of Artificial Sequence Synthetic peptide”
GETCLLDILDTAVQEEYSAMRDQYM

SEQ ID NO: 53 - protein, 25 aa, Artificial Sequence
  source: /note=“Description of Artificial Sequence Synthetic peptide”
ETCLLDILDTAGREEYSAMRDQYMR

SEQ ID NO: 54 - protein, 25 aa, Artificial Sequence
  source: /note=“Description of Artificial Sequence Synthetic peptide”
ETCLLDILDTAGHEEYSAMRDQYMR

SEQ ID NO: 55 - protein, 25 aa, Artificial Sequence
  source: /note=“Description of Artificial Sequence Synthetic peptide”
ETCLLDILDTAGKEEYSAMRDQYMR

SEQ ID NO: 56 - protein, 25 aa, Artificial Sequence
  source: /note=“Description of Artificial Sequence Synthetic peptide”
ETCLLDILDTAGLEEYSAMRDQYMR

SEQ ID NO: 57 - protein, 25 aa, Artificial Sequence
  source: /note=“Description of Artificial Sequence Synthetic peptide”
ETCLLDILDTAGPEEYSAMRDQYMR

SEQ ID NO: 58 - protein, 25 aa, Artificial Sequence
  source: /note=“Description of Artificial Sequence Synthetic peptide”
MTEYKLVVVGAGRVGKSALTIQLIQ

SEQ ID NO: 59 - protein, 25 aa, Artificial Sequence
  source: /note=“Description of Artificial Sequence Synthetic peptide”
MTEYKLVVVGAGCVGKSALTIQLIQ

SEQ ID NO: 60 - protein, 25 aa, Artificial Sequence
  source: /note=“Description of Artificial Sequence Synthetic peptide”
MTEYKLVVVGAGDVGKSALTIQLIQ

SEQ ID NO: 61 - protein, 25 aa, Artificial Sequence
  source: /note=“Description of Artificial Sequence Synthetic peptide”
MTEYKLVVVGAGSVGKSALTIQLIQ

SEQ ID NO: 62 - protein, 25 aa, Artificial Sequence
  source: /note=“Description of Artificial Sequence Synthetic peptide”
MTEYKLVVVGAGVVGKSALTIQLIQ

SEQ ID NO: 63 - protein, 25 aa, Artificial Sequence
  source: /note=“Description of Artificial Sequence Synthetic peptide”
MTEYKLVVVGAVGVGKSALTIQLIQ

SEQ ID NO: 64 - protein, 25 aa, Artificial Sequence
  source: /note=“Description of Artificial Sequence Synthetic peptide”
MTEYKLVVVGAAGVGKSALTIQLIQ

SEQ ID NO: 65 - protein, 25 aa, Artificial Sequence
  source: /note=“Description of Artificial Sequence Synthetic peptide”
MTEYKLVVVGACGVGKSALTIQLIQ

SEQ ID NO: 66 - protein, 25 aa, Artificial Sequence
  source: /note=“Description of Artificial Sequence Synthetic peptide”
MTEYKLVVVGADGVGKSALTIQLIQ

SEQ ID NO: 67 - protein, 25 aa, Artificial Sequence
  source: /note=“Description of Artificial Sequence Synthetic peptide”
MTEYKLVVVGARGVGKSALTIQLIQ

SEQ ID NO: 68 - protein, 25 aa, Artificial Sequence
  source: /note=“Description of Artificial Sequence Synthetic peptide”
MTEYKLVVVGASGVGKSALTIQLIQ

SEQ ID NO: 69 - protein, 25 aa, Artificial Sequence
  source: /note=“Description of Artificial Sequence Synthetic peptide”
DSDDVPMVLVGNNCDLAARTVESRQ

SEQ ID NO: 70 - protein, 189 aa, Artificial Sequence
  source: /note=“Description of Artificial Sequence Synthetic polypeptide”
MTEYKLVVVGADDVGKSAFTIQLIQNHFVDEYDPTIEDSYRKQVVIDGETCLLDILDTTDHEEYSAMRDQYMRTGEGFLCVFAINNTKSFEDIHHYREQIKRVKDSEDVPMVLVGNNCDLPSRTVDTKQAQDLARSYGIPFIETSTKTRQRVEDAFYTLVREIRQYRLKKISKEEKTPGCVKIKKCIIM

SEQ ID NO: 71 - protein, 25 aa, Artificial Sequence
  source: /note=“Description of Artificial Sequence Synthetic peptide”
QNAADSYSWVPEQAESRAMENQYSP

SEQ ID NO: 72 - protein, 20 aa, Artificial Sequence
  source: /note=“Description of Artificial Sequence Synthetic peptide”
KHKHHKHHKHHKHHKHHKHK

SEQ ID NO: 73 - protein, 17 aa, Artificial Sequence
  source: /note=“Description of Artificial Sequence Synthetic peptide”
KHHHKHHHKHHHKHHHK

SEQ ID NO: 74 - protein, 18 aa, Artificial Sequence
  source: /note=“Description of Artificial Sequence Synthetic peptide”
KHHHKHHHKHHHHKHHHK

SEQ ID NO: 75 - protein, 18 aa, Artificial Sequence
  source: /note=“Description of Artificial Sequence Synthetic peptide”
  MOD_RES (1)..(1): D-Lysine
  MOD_RES (5)..(5): D-Lysine
  MOD_RES (9)..(9): D-Lysine
  MOD_RES (14)..(14): D-Lysine
  MOD_RES (18)..(18): D-Lysine
XHHHXHHHXHHHHXHHHX

SEQ ID NO: 76 - protein, 19 aa, Artificial Sequence
  source: /note=“Description of Artificial Sequence Synthetic peptide”
HKHHHKHHHKHHHHKHHHK

SEQ ID NO: 77 - protein, 20 aa, Artificial Sequence
  source: /note=“Description of Artificial Sequence Synthetic peptide”
HHKHHHKHHHKHHHHKHHHK

SEQ ID NO: 78 - protein, 21 aa, Artificial Sequence
  source: /note=“Description of Artificial Sequence Synthetic peptide”
KHHHHKHHHHKHHHHKHHHHK

SEQ ID NO: 79 - protein, 18 aa, Artificial Sequence
  source: /note=“Description of Artificial Sequence Synthetic peptide”
KHHHKHHHKHHHKHHHHK

SEQ ID NO: 80 - protein, 18 aa, Artificial Sequence
  source: /note=“Description of Artificial Sequence Synthetic peptide”
KHHHKHHHHKHHHKHHHK

SEQ ID NO: 81 - protein, 19 aa, Artificial Sequence
  source: /note=“Description of Artificial Sequence Synthetic peptide”
KHHHKHHHHKHHHKHHHHK

SEQ ID NO: 82 - protein, 4 aa, Artificial Sequence
  source: /note=“Description of Artificial Sequence Synthetic peptide”
HHHK

SEQ ID NO: 83 - protein, 10 aa, Artificial Sequence
  source: /note=“Description of Artificial Sequence Synthetic peptide”
GGSGGGGSGG

SEQ ID NO: 84 - protein, 10 aa, Artificial Sequence
  source: /note=“Description of Artificial Sequence Synthetic peptide”
GGSGGGGSGG

SEQ ID NO: 85 - protein, 10 aa, Artificial Sequence
  source: /note=“Description of Artificial Sequence Synthetic peptide”
GGSLGGGGSG

SEQ ID NO: 86 - protein, 20 aa, Unknown
  source: /note=“Description of Unknown IL-2 signal peptide sequence”
MYRMQLLSCIALSLALVTNS

SEQ ID NO: 87 - protein, 15 aa, Severe acute respiratory syndrome coronavirus 2
MFVFLVLLPLVSSQC

SEQ ID NO: 88 - RNA, 615 nt, Artificial Sequence
  source: /note=“Description of Artificial Sequence Synthetic polynucleotide”
auguuuguuuuucuuguuuuauugccacuagucucuagucaguguaugacugaauauaaacuugugguaguuggagcugaugacguaggcaagagugccuuuacgauacagcuaauucagaaucauuuuguggacgaauaugauccaacaauagaggauuccuacaggaagcaaguaguaauugauggagaaaccugucucuuggauauucucgacacaacagaucacgaggaguacagugcaaugagggaccaguacaugaggacuggggagggcuuucuuuguguauuugccauaaauaauacuaaaucauuugaagauauucaccauuauagagaacaaauuaaaagaguuaaggacucugaagauguaccuaugguccuaguaggaaauaauugugauuugccuucuagaacaguagacacaaaacaggcucaggacuuagcaagaaguuauggaauuccuuuuauugaaacaucaacaaagacaagacagagaguggaggaugcuuuuuauacauuggugagagagauccgacaauacagauugaaaaaaaucagcaaagaagaaaagacuccuggcugugugaaaauuaaaaaaugcauuauaauguaa

SEQ ID NO: 89 - RNA, 59 nt, Artificial Sequence
  source: /note=“Description of Artificial Sequence Synthetic oligonucleotide”
aggacauuugcuucugacacaacuguguucacuagcaaccucaaacagacaccgccacc

SEQ ID NO: 90 - RNA, 162 nt, Artificial Sequence
  source: /note=“Description of Artificial Sequence Synthetic polynucleotide”
gcucgcuuucuugcuguccaauuucuauuaaagguuccuuuguucccuaaguccaacuacuaaacugggggauauuaugaagggccuugagcaucuggauucugccuaauaaaaaacauuuauuuucauugccaauaggccgaaaucggcaagcgcgaucgc

SEQ ID NO: 91 - DNA, 3335 bp, Artificial Sequence
  source: /note=“Description of Artificial Sequence Synthetic polynucleotide”
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagctttaatacgactcactataaggacatttgcttctgacacaactgtgttcactagcaacctcaaacagacaccgccaccatgtttgtttttcttgttttattgccactagtctctagtcagtgtatgactgaatataaacttgtggtagttggagctgatgacgtaggcaagagtgcctttacgatacagctaattcagaatcattttgtggacgaatatgatccaacaatagaggattcctacaggaagcaagtagtaattgatggagaaacctgtctcttggatattctcgacacaacagatcacgaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataattgtgatttgccttctagaacagtagacacaaaacaggctcaggacttagcaagaagttatggaattccttttattgaaacatcaacaaagacaagacagagagtggaggatgctttttatacattggtgagagagatccgacaatacagattgaaaaaaatcagcaaagaagaaaagactcctggctgtgtgaaaattaaaaaatgcattataatgtaagctcgctttcttgctgtccaatttctattaaaggttcctttgttccctaagtccaactactaaactgggggatattatgaagggccttgagcatctggattctgcctaataaaaaacatttattttcattgccaataggccgaaatcggcaagcgcgatcgcaaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaagaattcctcgaggcgcgcccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 92 - RNA, 132 nt, Artificial Sequence
  source: /note=“Description of Artificial Sequence Synthetic polynucleotide”
gcucgcuuucuugcuguccaauuucuauuaaagguuccuuuguucccuaaguccaacuacuaaacugggggauauuaugaagggccuugagcaucuggauucugccuaauaaaaaacauuuauuuucauugc

SEQ ID NO: 93 - RNA, 55 nt, Artificial Sequence
  source: /note=“Description of Artificial Sequence Synthetic oligonucleotide”
ggcgcucgagcagguucagaaggagaucaaaaacccccaaggaucaaacgccacc

SEQ ID NO: 94 - RNA, 50 nt, Artificial Sequence
  source: /note=“Description of Artificial Sequence Synthetic oligonucleotide”
gggcgcucgagcagguucagaaggagaucaaaaacccccaaggaucaaac

SEQ ID NO: 95 - RNA, 50 nt, Artificial Sequence
  source: /note=“Description of Artificial Sequence Synthetic oligonucleotide”
acauuugcuucugacacaacuguguucacuagcaaccucaaacagacacc

SEQ ID NO: 96 - RNA, 55 nt, Artificial Sequence
  source: /note=“Description of Artificial Sequence Synthetic oligonucleotide”
ggcgcacgagcagggagagaaggagaucaaaaacccccaaggaucaaacgccacc

SEQ ID NO: 97 - RNA, 121 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaagaaaaaaaaaaaaaaaaaaaaaaaaaaaaaaaaaaaaaaaaaaaaaaaaaaaaaaaaaaaa

SEQ ID NO: 98 - RNA, 48 nt, Artificial Sequence
  source: /note=“Description of Artificial Sequence Synthetic oligonucleotide”
cggcaauaaaaagacagaauaaaacgcacgguguugggucguuuguuc

SEQ ID NO: 99 - protein, 8 aa, Artificial Sequence
  source: /note=“Description of Artificial Sequence Synthetic peptide”
  VARIANT (2)..(2): /replace=“Ile”
  MOD_RES (4)..(4): Any amino acid
  SITE (1)..(8): /note=“Variant residues given in the sequence have no preference with respect to those in the annotations for variant positions”
DVEXNPGP

SEQ ID NO: 100 - protein, 75 aa, Artificial Sequence
  source: /note=“Description of Artificial Sequence Synthetic polypeptide”
  SITE (1)..(75): /note=“This sequence may encompass 1-15 ′Gly Gly Gly Gly Ser′ repeating units”
GGGGSGGGGSGGGGSGGGGSGGGGSGGGGSGGGGSGGGGSGGGGSGGGGSGGGGSGGGGSGGGGSGGGGSGGGGS

SEQ ID NO: 101 - protein, 189 aa, Homo sapiens [sequence omitted]

SEQ ID NO: 102 - protein, 188 aa, Homo sapiens [sequence omitted]

SEQ ID NO: 103 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 104 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 105 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 106 - RNA, 300 nt, Artificial Sequence
  source: /note=“Description of Artificial Sequence Synthetic polynucleotide”
  misc_feature (1)..(300): /note=“This sequence may encompass 10-300 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0026317 A1
(43) Pub. Date: Jan. 25, 2024

(54) PAN-RAS mRNA CANCER VACCINES

(71) Applicants: Dong SHEN, Gaithersburg, MD (US); David BROWN, Gaithersburg, MD (US); Renxiang CHEN, Gaithersburg, MD (US); RNAimmune, Inc, Gaithersburg, MD (US)

(72) Inventors: Dong Shen, Gaithersburg, MD (US); David Brown, Gaithersburg, MD (US); Renxiang Chen, Gaithersburg, MD (US)

(21) Appl. No.: 18/249,023

(22) PCT Filed: Oct. 13, 2021
(86) PCT No.: PCT/US2021/054859

§ 371 Date: Apr. 13, 2023

Related U.S. Application Data
(60) Provisional application No. 63/091,711, filed on Oct. 14, 2020.

(51) Int. Cl. C12N 9/14; C12P 19/30; A61K 39/00; A61K 9/127; A61K 47/69; A61P 35/00; A61K 47/54

(52) U.S. Cl. CPC C12N 9/14; C12Y 306/05002; C12P 19/30; A61K 39/001164; A61K 9/1273; A61K 47/6911; A61P 35/00; A61K 47/54; A61K 2039/53; A61K 2039/585; A61K 2039/55555

(57) ABSTRACT

Compositions and methods are provided for potent mRNA vaccines for treatment of cancers with a mutation in the ras gene family. The compositions include a pharmaceutical composition comprising, or consisting essentially of, or yet further comprising mRNA molecules encoding at least one of multiple peptides of a group of somatic mutants and a pharmaceutically acceptable carrier. Methods for stimulating system immune responses and treatment are provided, including intratumoral, intravenous, intramuscular, intradermal, or subcutaneous injection of the composition as disclosed herein.

### CROSS-REFERENCE TO RELATED APPLICATION

This application claims priority under 35 U.S.C. § 119(e) of U.S. Provisional Application No. 63/091,711, filed Oct. 14, 2020, the contents of which are incorporated by reference in its entirety into the present application.

### FIELD OF THE DISCLOSURE

This disclosure is related to therapeutic vaccines for treating cancer patients bearing mutations in tumor-suppressor genes such as the Pan-RAS family of genes.

### BACKGROUND

Cancer is a family of genetic disorders that changes of genetic material drive a normal cell into a dysregulated state that manifests as malignant growth of tumor tissues. With aging of the society, cancer poses an increasing burden both in mortality and healthcare cost. According to data from the National Cancer Institute (NCI), in 2020, roughly 1.8 million people will be diagnosed with cancer and an estimated 606,520 people will die of cancer in the United States. Of all different types of cancers, lung cancer is responsible for the most deaths with 135,720 people expected to die from this disease. That is nearly three times the 53,200 deaths due to colorectal cancer, which is the second most common cause of cancer death. Pancreatic cancer is the third deadliest cancer, causing 47,050 deaths.

Thus, a need exists to treat a cancer effectively. This disclosure satisfies this need and provides related advantages as well.

### SUMMARY

In one aspect, provided herein is a composition or a vaccine that comprises, or consists essentially of, or yet further consists of a messenger ribonucleic acid (mRNA) molecule that expresses cancer neoantigens that are derived from mutated human ras genes. In another aspect, they are formulated with a carrier, e.g., they are formulated with a pharmaceutically acceptable carrier. Suitable carriers include, but are not limited to, Histidine-Lysine Co-polymers (HKP), 4-(dimethylamino)-butanoic acid, (10Z,13Z)-1-(9Z,12Z)-9,12-octadecadien-1-yl-10,13-nonadecadien-1-yl ester (DLIN-MC3-DMA or MC3), 1,2-Dioleoyl-3-trimethylammonium propane (DOTAP), or any combination thereof, which in some aspects, can serve as an adjuvant for amplifying an immune response against cancer cells that harbor mutations in ras. Methods also are provided for using these pharmaceutical compositions, including methods of treatment, process development, and specific delivery routes.

In one aspect, provided is a ribonucleic acid (RNA) comprising, or consisting essentially of, or yet further consisting of an open reading frame (ORF) encoding one or more ras derived peptides. In some embodiments, each of the one or more ras derived peptides consists of between 23 and 29 amino acid residues, for example 25 amino acid residues. Additionally or alternatively, the encoded peptides are selected from the group as set forth in SEQ ID NOs:1 to 69, or an equivalent of each thereof. In some embodiments, the one or more ras derived peptides do not comprise or alternatively consist essentially of, or alternatively consisting of any one or more of SEQ ID NOs: 1-18, 32-49 or 53-68.

In another aspect, provided is an isolated ribonucleic acid (RNA) comprising, or consisting essentially of, or yet further consisting of an open reading frame (ORF) encoding a ras derived peptide. In some embodiments, the encoded ras derived peptide comprises one or more (for example, any one, or any two, or any three, or any four, or all five) of the following mutations: a phenylalanine (F) aligned to the 19th amino acid residue of SEQ ID NO: 70 (referred to herein as L19F); a threonine (T) aligned to the 59th amino acid residue of SEQ ID NO: 70 (referred to herein as A59T); an aspartic acid (D) aligned to the 60th amino acid residue of SEQ ID NO: 70 (referred to herein as G60D); an asparagine (N) aligned to the 117th amino acid residue of SEQ ID NO: 70 (referred to herein as K117N); or a T aligned to the 146th amino acid residue of SEQ ID NO: 70 (referred to herein as A146T). In some embodiments, the encoded ras derived peptide further comprises any one or more (for example, any one, or any two, or any three) of the following mutations: a D aligned to the 12th amino acid residue of SEQ ID NO: 70 (referred to herein as a G12D); a D aligned to the 13th amino acid residue of SEQ ID NO: 70 (referred to herein as G13D); or a histidine (H) aligned to the 61th amino acid residue of SEQ ID NO: 70 (referred to herein as Q61H). In some embodiments, the encoded ras derived peptide comprises the following mutations: G12D, G13D, L19F, A59T, G60D, Q61H, K117N, and A146T. In some embodiments, the encoded ras derived peptide comprises, or consists essentially of, or yet further consists of the polypeptide as set forth in SEQ ID NO: 70 or an equivalent thereof, with the proviso that the equivalent retains the eight mutations of G12D, G13D, L19F, A59T, G60D, Q61H, K117N, and A146T. In some embodiments, the RNA comprises, or consists essentially of, or yet further consists of the polynucleotide as set forth in SEQ ID NO: 88 or nucleotide (nt) 1 to nt 612 of SEQ ID NO: 88. In further embodiments, the RNA is formulated in a pharmaceutically acceptable carrier, such as encapsulated in a nanoparticle.

In a further aspect, provided is a polynucleotide (such as a DNA) encoding an RNA as disclosed herein, or a polynucleotide complementary thereto, or both. In yet a further aspect, provided is a vector comprising, or consisting essentially of, or yet further consisting of a polynucleotide as disclosed herein. In some embodiments, the vector further comprises a regulatory sequence operatively linked to the polynucleotide to direct the replication or transcription thereof, such as a promoter. In some embodiments, the vector is a non-viral vector, such as a plasmid, a liposome, or a micelle. In further embodiments, the vector is pUC57, or pSFV1, or pcDNA3, or pTK126. In yet further embodiments, the vector comprises, or consists essentially of, or yet further consist of the polynucleotide as set forth in SEQ ID NO: 91 or an equivalent thereof which transcribes to the same RNA. In some embodiments, the vector is a viral vector, such as an adenoviral vector, or an adeno-associated viral vector, or a retroviral vector, or a lentiviral vector, or a plant viral vector.

In one aspect, provided is a cell comprising one or more of: an RNA as disclosed herein, a polynucleotide as disclosed herein, or a vector as disclosed herein. In one aspect, the cell is a prokaryotic cell. In another aspect, the cell is a eukaryotic cell.

In a further aspect, provided is a composition comprising, or consisting essentially of, or yet further consisting of a carrier (e.g., a pharmaceutically acceptable carrier) and one or more of: an RNA as disclosed herein, a polynucleotide as disclosed herein, a vector as disclosed herein, or a cell as disclosed herein.

In yet a further aspect, provided is a method of producing an RNA of this disclosure. In some embodiments, the method comprises, or consists essentially of, or yet further consists of culturing a cell as disclosed herein under conditions suitable for expressing the RNA (such as transcribing a DNA to the RNA). In one aspect, the cell comprises the DNA encoding the RNA of this disclosure. In some embodiments, the method comprises, or consists essentially of, or yet further consists of contacting a polynucleotide as disclosed herein or a vector as disclosed herein with an RNA polymerase, adenosine triphosphate (ATP), cytidine triphosphate (CTP), guanosine-5′-triphosphate (GTP), and uridine triphosphate (UTP) or a chemically modified UTP under conditions suitable for expressing the RNA (such as transcribing a DNA to the RNA). In further embodiments, the method further comprises isolating the RNA. Additionally provided is an RNA produced by a method as disclosed herein.

Additionally provided is a composition (such as an immunogenic composition) comprising, or consisting essentially of, or yet further consisting of an effective amount of an RNA as disclosed herein formulated in a carrier, e.g., a pharmaceutically acceptable carrier, such as a nanoparticle. In some embodiments, the nanoparticle is a polymeric nanoparticle carrier, for example those comprising, or consisting essentially of, or yet further consisting of a Histidine-Lysine co-polymer (HKP), such as H3K(+H)4b or H3k(+H)4b or both. In some embodiments, the nanoparticle is a lipid nanoparticle, for example, 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof.

In one aspect, provided is a method of producing a composition (such as an immunogenic composition) as disclosed herein. The method comprises, or consists essentially of, or yet further consists of contacting an RNA as disclosed herein with an HKP or a lipid or both, thereby the RNA and the HKP or lipid or both HKP and lipid are self-assembled into nanoparticles.

In another aspect, provided is a method of treating a subject having a cancer or suspect of having a cancer, or at risk or alternatively a high risk of having a cancer. The method comprises, or consists essentially of, or yet further consists of administering to the subject, for example a pharmaceutically effective amount of, any one or more of: an RNA as disclosed herein, a polynucleotide as disclosed herein, a vector as disclosed herein, a cell as disclosed herein, or a composition (such as an immunogenic composition) as disclosed herein. In some embodiments, the cancer comprises (such as expresses) one or more mutations (also referred to herein as neoantigens) expressed by an RNA as disclosed herein, such as a ras mutation. In some embodiments, the cancer comprises a mutated ras gene encoding a neoantigen as disclosed herein. In further embodiments, the method further comprises, or consists essentially of, or yet further consists of administering to the subject an additional anti-cancer therapy.

In yet another aspect, provided is a kit for use in a method as disclosed herein. The kit comprises, or consists essentially of, or yet further consists of instructions for use and one or more of: an RNA as disclosed herein, a polynucleotide as disclosed herein, a vector as disclosed herein, a cell as disclosed herein, a composition as disclosed herein, a pharmaceutically acceptable carrier as disclosed herein, or an anti-cancer therapy.

### BRIEF DESCRIPTION OF THE DRAWINGS

FIG. 1 illustrates the major protein domains of ras.

FIG. 2 lists advantages and disadvantages of viral vectored vaccines, DNA vaccines and RNA vaccines.

FIG. 3 illustrates screening neoantigen by in silico prediction and in vitro assays.

FIG. 4 illustrates a minigene structure of an mRNA vaccine. An exemplified amino acid sequence encoded by the minigene, QNAADSYSWVPEQAESRAMENQYSP, is provided herein as SEQ ID NO: 71.

FIG. 5 provides a schematic representation of an optimized mRNA vaccine expression structure. This structure can be contained and/or transcribed in a linear in vitro transcription (IVT) expression system or plasmid DNA delivery vector.

FIG. 6 illustrates plasmid vectors which can be used for mRNA production. The commonly used plasmids are pSFV1, pcDNA3 and pTK126.

FIG. 7 illustrates polylipid nanoparticle (PLNP) and lipid nanoparticle (LNP) structures.

FIG. 8 shows that H3K(+H)4b is a significantly better carrier than H3K4b in mRNA delivery. To form polyplexes, the mRNA (1 g) was mixed with 3 different ratios of the HK (4, 8, 12 g) polymer for 30 minutes. The mRNA was then added to the cells for 24 h before the luciferase activity was measured. H3K(+H)4b vs H3K4b, P<0.0001 and P<0.001 respectively.

FIG. 9 shows that H3K(+H)4b binds to mRNA more tightly than H3K4b. The retardation electrophoresis assay of H3K(+H)4b and H3K4b at the various ratios of mRNA and polypeptide (wt: wt; peptide: mRNA) in the 1% agarose gel. Lane 1 and Lane 7: mRNA alone (1 μg). Other Lanes: weight ratios of mRNA and polypeptide.

FIG. 10 shows that HK carriers with extra histidine in the second motif have improved performance in mRNA transfection comparing H3K(+H)4b with other 4-branched HK peptides for mRNA transfection. H3K(+H)4b was also compared to HK peptides without additional histidine in the second motif. H3k(+H)4b was the most effective peptide carrier of mRNA (H3k(+H)4b vs H3K(+4b); *, P<0.05).

FIG. 11 shows transfection of MDA-MB-231 cells with a combination of DOTAP and HK peptides. The combination of DOTAP and HK peptides significantly increased the expression rate of mRNA in cells.

FIG. 12 provides a comparison of mRNA transfection with DOTAP and several HK peptides. DOTAP combined with H3k(+H)4b is the most efficient in mRNA transfection.

FIG. 13 provides exemplified structures of Spermine-Lipid Conjugates (SLiC) species.

FIG. 14 provides an alignment among the sequences set forth in SEQ ID NOs: 70, 101, 103 and 104 performed using Clustal Omega accessible at www.ebi.ac.uk/Tools/msa/clustalo/.

FIG. 15 shows in vitro expression of RAS. ELISA was used to detect Ras antibodies (Ab) generated in immunized mice. A His-tagged Ras protein was used as the ELISA antigen. Various mRNA formulations of a RAS cancer vaccine candidate were used to immunize mice.

FIG. 16 illustrates 8 mutational hotspots in RAS.

FIG. 17 provides RAS expression confirmed using western blot. Expression of β-action was measured and served as a loading control.

FIG. 18 shows in vitro RAS expression in cells transfected with HKP(H) formulated nanoparticles. Expression of β-action was measured and served as a loading control.

FIG. 19 shows in vitro RAS expression in cells transfected with LNP formulated nanoparticles. Expression of β-action was measured and served as a loading control.

FIG. 20 illustrates an in vivo animal model evaluating a composition as disclosed herein. Briefly, mice were immunized on Day 0 and a booster immunization was given on Day 28. Sera were collected on Day 28 and Day 42. Anti-RAS antibodies in the sera were then assessed. After sacrificing the mice, spleens were removed and evaluated using qRT-PCR.

FIG. 21 plots ELISA data detecting the anti-RAS antibodies in sera.

FIGS. 22A to 22D show results evaluating IgG isotypes (FIG. 22A, IgG2a; FIG. 22B, IgG2b; FIG. 22C, IgG1; and FIG. 22D, IgG3) in mice immunized with the Ras vaccine. The dominant IgG isotype in mice immunized with Ras vaccine is IgG2b.

FIGS. 23A to 23B provide expression result of Th1 (FIG. 23A) and Th2 (FIG. 23B) related genes assessed using qRT-PCR.

FIGS. 24A to 24C provide NGS results of mice immunized with the RAS vaccine. RNAs from spleen were isolated from 6 mice, and sent for NGS analysis. NGS was done using the RNAs from mice #1, #2, #3, and #5. Such mouse numbering correlates with the numbering in FIGS. 21-23. Based on ELISA results, #5 mouse was used as relative negative control. Accordingly, FIG. 24A shows the NGS result from mouse #1 compared to that of mouse #5; FIG. 24B shows the NGS result from mouse #2 compared to that of mouse #5; and FIG. 24C shows the NGS result from mouse #3 compared to that of mouse #5.

FIGS. 25A to 25C provide the top 20 KEGG pathways shown by NGS of mice immunized with the RAS vaccine. In the figure titles, mouse #1 as identified in FIGS. 21-23 is noted as “LNP-1”, mouse #2 as identified in FIGS. 21-23 is noted as “LNP_2”, mouse #3 as identified in FIGS. 21-23 is noted as “HKPH”, and mouse #5 as identified in FIGS. 21-23 is noted as “HKP”. Accordingly, FIG. 25A lists the top 20 KEGG pathways identified using the sample from mouse #1; FIG. 25B lists the top 20 KEGG pathways identified using the sample from mouse #2; and FIG. 25C lists the top 20 KEGG pathways identified using the sample from mouse #3.

FIGS. 26A to 26B provide up-regulated genes revealed by NGS in mice immunized with the RAS vaccine. In the figure legends, mouse #1 as identified in FIGS. 21-23 is noted as “LNP-1”, mouse #2 as identified in FIGS. 21-23 is noted as “LNP_2”, mouse #3 as identified in FIGS. 21-23 is noted as “HKPH”, and mouse #5 as identified in FIGS. 21-23 is noted as “HKP”. FIG. 26A shows pathways of Th1 and Th2 differentiation. Six genes are marked with boxes in FIG. 26A and their expression levels are further plotted in FIG. 26B using FKPM counts. FKPM (short for the expected number of Fragments Per Kilobase of transcript sequence per Millions base pairs sequenced) is the most common method of estimating gene expression levels.

FIGS. 27A to 27B provide pathway analysis revealed by NGS in mice immunized with the RAS vaccine. In the figure legends, mouse #1 as identified in FIGS. 21-23 is noted as “LNP-1”, mouse #2 as identified in FIGS. 21-23 is noted as “LNP_2”, mouse #3 as identified in FIGS. 21-23 is noted as “HKPH”, and mouse #5 as identified in FIGS. 21-23 is noted as “HKP”. FIG. 27A shows FKPM counts of genes involved in the antigen processing and presentation pathway as illustrated in FIG. 27B.

FIG. 28 plots gene expression levels shown as FKPM counts of Th1 and Th2 related genes assessed using NGS. In the figure legends, mouse #1 as identified in FIGS. 21-23 is noted as “LNP-1”, mouse #2 as identified in FIGS. 21-23 is noted as “LNP_2”, mouse #3 as identified in FIGS. 21-23 is noted as “HKPH”, and mouse #5 as identified in FIGS. 21-23 is noted as “HKP”.

FIG. 29 plots gene expression levels shown as FKPM counts of CTLA-4 and LFA-1 assessed using NGS. CTLA-4 and LFA-1 are phenotypic markers for activated CD8+ cells. In the figure legends, mouse #1 as identified in FIGS. 21-23 is noted as “LNP-1”, mouse #2 as identified in FIGS. 21-23 is noted as “LNP_2”, mouse #3 as identified in FIGS. 21-23 is noted as “HKPH”, and mouse #5 as identified in FIGS. 21-23 is noted as “HKP”.

FIGS. 30A to 30B show immunization with the LNP-RAS vaccine reduces tumor growth in vivo. FIG. 30A plots sizes of the tumors in mm 3 while FIG. 30B plots weights of the tumor in g.

FIG. 31 illustrates a further in vivo animal model evaluating a composition as disclosed herein. Briefly, mice were immunized on Day 0 and a booster immunization was given on Day 14. Blood was collected on Day 21. And the tumor cells were inoculated on Day 28.

FIG. 32 shows ELISA results detecting anti-RAS antibodies in mouse sera.

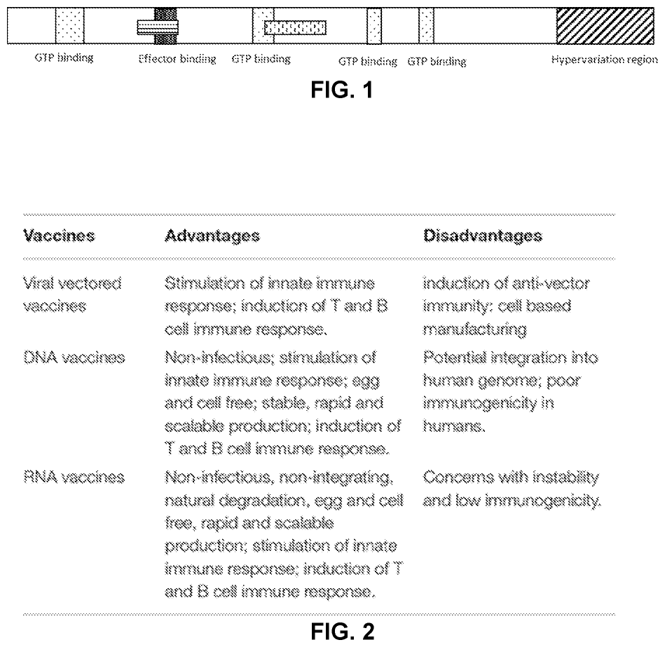

### DETAILED DESCRIPTION

### Definitions

As it would be understood, the section or subsection headings as used herein is for organizational purposes only and are not to be construed as limiting and/or separating the subject matter described.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press); and Plotkin et al., Plotkin; Human Vaccines, 7^{th} edition (Elsevier).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

In some embodiments, the terms “first” “second” “third” “fourth” or similar in a component name are used to distinguish and identify more than one components sharing certain identity in their names. For example, “first RNA” and “second RNA” are used to distinguishing two RNAs.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

In some embodiments, a fragment of a protein can be an immunogenic fragment. As used herein, the term “immunogenic fragment” refers to such a polypeptide fragment, which at least partially retains the immunogenicity of the protein from which it is derived. In some embodiments, the immunogenic fragment is at least about 3 amino acid (aa) long, or at least about 4 aa long, or at least about 5 aa long, or at least about 6 aa long, or at least about 7 aa long, or at least about 8 aa long, or at least about 9 aa long, or at least about 10, aa long, or at least about 15, aa long, or at least about 20 aa long, or at least about 25 aa long, or at least about 30 aa long, or at least about 35 aa long, or at least about 40 aa long, or at least about 50 aa long, or at least about 60 aa long, or at least about 70 aa long, or at least about 80 aa long, or at least about 90 aa long, or at least about 100 aa long, or at least about 120 aa long, or at least about 150 aa long, or at least about 200, or longer.

As used herein, an amino acid (aa) or nucleotide (nt) residue position in a sequence of interest “corresponding to” or “aligned to” an identified position in a reference sequence refers to that the residue position is aligned to the identified position in a sequence alignment between the sequence of interest and the reference sequence. Various programs are available for performing such sequence alignments, such as Clustal Omega and BLAST. In one aspect, equivalent polynucleotides, proteins and corresponding sequences can be determined using BLAST (accessible at blast.ncbi.nlm.nih.gov/Blast.cgi, last accessed on Aug. 1, 2021).

As used herein, an amino acid mutation is referred to herein as two letters separated by an integer, such as L19F. The first letter provides the one letter code of the original amino acid residue to be mutated; while the last letter provides the mutation, such as A indicating a deletion, or one letter code of the mutated amino acid residue. In some embodiments, the integer is the numbering of the to-be-mutated amino acid residue in the amino acid sequence free of the mutation, optionally counting from the N terminus to the C terminus. In some embodiments, the integer is the numbering of the mutated amino acid residue in the mutated amino acid sequence, optionally counting from the N terminus to the C terminus.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively at least about 96% homology or identity, or alternatively at least about 97% homology or identity, or alternatively at least about 98% homology or identity, or alternatively at least about 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

An equivalent of a reference polypeptide comprises, consists essentially of, or alternatively consists of an polypeptide having at least 80%, or at least 85%, or at least 90%, or at least 95%, or at least about 96%, or at least 97%, or at least 98%, or at least 99% amino acid identity to the reference polypeptide (as determined, in one aspect using the Clustal Omega alignment program), or a polypeptide that is encoded by a polynucleotide that hybridizes under conditions of high stringency to the complementary sequence of a polynucleotide encoding the reference polypeptide, optionally wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some embodiments, a first sequence (nucleic acid sequence or amino acid) is compared to a second sequence, and the identity percentage between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

In some embodiments, an equivalent of a reference polypeptide comprises, or consists essentially of, or yet further consists of the reference polypeptide with one or more amino acid residues replaced by a conservative substitution. The substitution can be “conservative” in the sense of being a substitution within the same family of amino acids. The naturally occurring amino acids can be divided into the following four families and conservative substitutions will take place within those families.
(1) Amino acids with basic side chains: lysine, arginine, histidine.
(2) Amino acids with acidic side chains: aspartic acid, glutamic acid
(3) Amino acids with uncharged polar side chains: asparagine, glutamine, serine, threonine, tyrosine.
(4) Amino acids with nonpolar side chains: glycine, alanine, valine, leucine, isoleucine, proline, phenylalanine, methionine, tryptophan, cysteine.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “RNA” as used herein refers to its generally accepted meaning in the art. Generally, the term RNA refers to a polynucleotide comprising at least one ribofuranoside moiety. The term can include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, for example at one or more nucleotides of the RNA. Nucleotides in the nucleic acid molecules can also comprise non-standard nucleotides, such as non-naturally occurring nucleotides or chemically synthesized nucleotides or deoxynucleotides. These altered RNAs can be referred to as analogs or analogs of naturally-occurring RNA. In some embodiments, the RNA is a messenger RNA (mRNA).

“Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some embodiments, an mRNA as disclosed herein comprises, or consists essentially of, or yet further consists of at least one coding region, a 5′ untranslated region (UTR), a 3′ UTR, a 5′ cap and a poly-A tail.

Vaccination is the most successful medical approach to disease prevention and control. The successful development and use of vaccines has saved thousands of lives and large amounts of money. A key advantage of RNA vaccines is that RNA can be produced in the laboratory from a DNA template using readily available materials, less expensively and faster than conventional vaccine production, which can require the use of chicken eggs or other mammalian cells. In addition, mRNA vaccines have the potential to streamline vaccine discovery and development, and facilitate a rapid response to emerging infectious diseases, see, for example, Maruggi et al., Mol Ther. 2019; 27(4):757-772.

Preclinical and clinical trials have shown that mRNA vaccines provide a safe and long-lasting immune response in animal models and humans. mRNA vaccines against infectious diseases may be developed as prophylactic or therapeutic treatments. mRNA vaccines expressing antigens of infectious pathogens have been shown to induce potent T cell and humoral immune responses. See, for example, Pardi et al., Nat Rev Drug Discov. 2018; 17:261-279. The production procedure to generate mRNA vaccines is cell-free, simple, and rapid, compared to production of whole microbe, live attenuated, and subunit vaccines. This fast and simple manufacturing process makes mRNA a promising bio-product that can potentially fill the gap between emerging infectious disease and the desperate need for effective vaccines.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, or protein, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, or protein, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “gene” refers to a polynucleotide containing at least one open reading frame (ORF) that is capable of encoding a particular polypeptide or protein after being transcribed and translated.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

The terms “chemical modification” and “chemically modified” refer to modification with respect to adenosine (A), guanosine (G), uridine (U), thymidine (T) or cytidine (C) ribonucleosides or deoxyribonucleosides in at least one of their position, pattern, percent or population. In some embodiments, the term refers to the ribonucleotide modifications in naturally occurring 5′-terminal mRNA cap moieties. In further embodiments, the chemical modification is selected from pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, or 2′-O-methyl uridine. In some embodiments the extent of incorporation of chemically modified nucleotides has been optimized for improved immune responses to the vaccine formulation. In other embodiments, the term excludes the ribonucleotide modifications in naturally occurring 5′-terminal mRNA cap moieties.

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of residues of the RNA is chemically modified by one or more of modifications as disclosed herein. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of uridine residues of the RNA is chemically modified by one or more of modifications as disclosed herein.

In some embodiments, an RNA as disclosed herein is optimized. Optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide.

A “3′ untranslated region” (3′ UTR) refers to a region of an mRNA that is directly downstream (i.e., 3′) from the stop codon (i.e., the codon of an mRNA transcript that signals a termination of translation) that does not encode a polypeptide. In some embodiments, a 3′ UTR as used herein comprises, or consists essentially of, or yet further consists of one or more of the following:

(SEQ ID NO: 92)
GCUCGCUUUCUUGCUGUCCAAUUUCUAUUAAAGGUUCCUUUGUUCCCU
AAGUCCAACUACUAAACUGGGGGAUAUUAUGAAGGGCCUUGAGCAUCU
GGAUUCUGCCUAAUAAAAAACAUUUAUUUUCAUUGC;
(SEQ ID NO: 93)
GGCGCUCGAGCAGGUUCAGAAGGAGAUCAAAAACCCCCAAGGAUCAAA
CGCCACC;
or
(SEQ ID NO: 94)
GGGCGCUCGAGCAGGUUCAGAAGGAGAUCAAAAACCCCCAAGGAUCAA
AC

A “5′ untranslated region” (5′ UTR) refers to a region of an RNA that is directly upstream (i.e., 5′) from the start codon (i.e., the first codon of an mRNA transcript translated by a ribosome) that does not encode a polypeptide. In some embodiments, a 5′ UTR as used herein comprises, or consists essentially of, or yet further consists of one or both of the following:

(SEQ ID NO: 95)
ACAUUUGCUUCUGACACAACUGUGUUCACUAGCAACCUCAAACAGACAC
C;
or
(SEQ ID NO: 96)
GGCGCACGAGCAGGGAGAGAAGGAGAUCAAAAACCCCCAAGGAUCAAA
CGCCACC

In some embodiments, an RNA further comprises a polyA tail. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. Additionally or alternatively, in a relevant biological setting (e.g., in cells, in vivo) the polyA tail functions to protect mRNA from enzymatic degradation, e.g., in the cytoplasm, and aids in transcription termination, export of the mRNA from the nucleus and translation. In some embodiments, a polyA tail as used herein comprises, or consists essentially of, or yet further consists of one or more of the following:

(SEQ ID NO: 97)
AAAAAAAAAAAAAAAAAAAAAAAAAAAAAAAAAAAAAAAAAAAAAAA
AAAAAAAAAAAAAGAAAAAAAAAAAAAAAAAAAAAAAAAAAAAAAAA
AAAAAAAAAAAAAAAAAAAAAAAAAAA;
or
(SEQ ID NO: 98)
CGGCAAUAAAAAGACAGAAUAAAACGCACGGUGUUGGGUCGUUUGUU
C.

In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et al., Methods Mol Biol. 703:29-41(2011); Rio et al. RNA: A Laboratory Manual. Cold Spring Harbor: Cold Spring Harbor Laboratory Press, 2011, 205-220; and Cooper, Geoffery M. The Cell: A Molecular Approach. 4th ed. Washington D.C.: ASM Press, 2007, 262-299). Generally, IVT utilizes a DNA template featuring a promoter sequence upstream of a sequence of interest. The promoter sequence is most commonly of bacteriophage origin (ex. the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that an RNA as disclosed herein can be made using any appropriate synthesis method. For example, in some embodiments, an RNA is made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide, T3 promoter complementary oligonucleotide, or SP6 promoter complementary oligonucleotide), followed by IVT using an RNA polymerase corresponding to the promoter sequence, e.g., a T7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles, e.g., 3 to 10 cycles. In some embodiments, a double-stranded DNA template is synthesized wholly or in part by chemical synthesis methods. The double-stranded DNA template can be subjected to in vitro transcription as described herein.

“Under transcriptional control”, which is also used herein as “directing expression of” or any grammatical variation thereof, is a term well understood in the art and indicates that transcription and optionally translation of a polynucleotide sequence, usually a DNA sequence, depends on its being operatively linked to an element which contributes to the initiation of, or promotes, transcription.

“Operatively linked” intends the polynucleotides are arranged in a manner that allows them to function in a cell.

The term “a regulatory sequence”, “an expression control element” or “promoter” as used herein, intends a polynucleotide that is operatively linked to a target polynucleotide to be transcribed or replicated, and facilitates the expression or replication of the target polynucleotide.

A promoter is an example of an expression control element or a regulatory sequence. Promoters can be located 5′ or upstream of a gene or other polynucleotide, that provides a control point for regulated gene transcription. In some embodiments, a promoter as used herein is corresponding to the RNA polymerase. In further embodiments, a promoter as sued herein comprises, or consists essentially of, or yet further consists of a T7 promoter, or a SP6 promoter, or a T3 promoter. Non-limiting examples of suitable promoters are provided in WO2001009377A1.

An “RNA polymerase” refers to an enzyme that produces a polyribonucleotide sequence, complementary to a pre-existing template polynucleotide (DNA or RNA). In some embodiments, the RNA polymerase is a bacteriophage RNA polymerase, optionally a T7 RNA polymerase, or a SP6 RNA polymerase, or a T3 RNA polymerase. Non-limiting examples of suitable polymerase are further detailed in U.S. Ser. No. 10/526,629B2.

In some embodiments, the term “vector” intends a recombinant vector that retains the ability to infect and transduce non-dividing and/or slowly-dividing cells and optionally integrate into the target cell's genome. Non-limiting examples of vectors include a plasmid, a nanoparticle, a liposome, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. In one embodiment, the viral vector is a retroviral vector. In one embodiment, the vector is a plasmid. In one embodiment, the vector is a nanoparticle, optionally a polymeric nanoparticle or a lipid nanoparticle.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

As used herein, the term “micelle” refers to a polymer assembly comprised of a hydrophilic shell (or corona) and a hydrophobic and/or ionic interior. In addition, the term micelle may refer to any poly ion complex assembly consisting of a multiblock copolymer possessing a net positive charge and a suitable negatively charged polynucleotide.

A “viral vector” is defined as a recombinantly produced virus or viral particle that comprises a polynucleotide to be delivered into a host cell, either in vivo, ex vivo or in vitro. As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. As used herein, Multiplicity of infection (MOI) refers to the number of viral particles that are added per cell during infection.

The term “adenovirus” is synonymous with the term “adenoviral vector” and refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (Ad11A and Ad 11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. The term bovine adenoviruses includes but is not limited to bovine adenovirus types 1, 2, 3, 4, 7, and 10. The term canine adenoviruses includes but is not limited to canine types 1 (strains CLL, Glaxo, R1261, Utrect, Toronto 26-61) and 2. The term equine adenoviruses includes but is not limited to equine types 1 and 2. The term porcine adenoviruses includes but is not limited to porcine types 3 and 4. In one embodiment of the invention, the adenovirus is derived from the human adenovirus serotypes 2 or 5. For purposes of this invention, adenovirus vectors can be replication-competent or replication deficient in a target cell. In some embodiments, the adenovirus vectors are conditionally or selectively replicating adenoviruses, wherein a gene(s] required for viral replication is/are operatively linked to a cell and/or context-specific promoter. Examples of selectively replicating or conditionally replicating viral vectors are known in the art (see, for example, U.S. Pat. No. 7,691,370).

A retrovirus such as a gammaretrovirus and/or a lentivirus comprises (a) envelope comprising lipids and glycoprotein, (b) a vector genome, which is an RNA (usually a dimer RNA comprising a cap at the 5′ end and a polyA tail at the 3′ end flanked by LTRs) derived to the target cell, (c) a capsid, and (d) proteins, such as a protease. U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

“Plant viruses” as used herein refers to a group of viruses that have been identified as being pathogenic to plants. These viruses rely on the plant host for replication, as they lack the molecular machinery to replicate without the plant host. Accordingly, a plant virus can be used as a vector for safely delivering a gene of interest to a non-plant animal subject. Plant viruses include but are not limited to tobacco mosaic virus, Maize chlorotic mottle virus; Maize rayado fino virus; Oat chlorotic stunt virus; Chayote mosaic tymovirus; Grapevine asteroid mosaic-associated virus; Grapevine fleck virus; Grapevine Red Globe virus; Grapevine rupestris vein feathering virus; Melon necrotic spot virus; Physalis mottle tymovirus; Prunus necrotic ringspot; Nigerian tobacco latent virus; Tobacco mild green mosaic virus; Tobacco necrosis virus; Eggplant mosaic virus; Kennedya yellow mosaic virus; Lycopersicon esculentum TVM viroid; Oat blue dwarf virus; Obuda pepper virus; Olive latent virus 1; Paprika mild mottle virus; PMMV; Tomato mosaic virus; Turnip vein-clearing virus; Carnation mottle virus; Cocksfoot mottle virus; Galinsoga mosaic virus; Johnsongrass chlorotic stripe mosaic virus; Odontoglossum ringspot virus; Ononis yellow mosaic virus; Panicum mosaic virus; Poinsettia mosaic virus; Pothos latent virus; or Ribgrass mosaic virus.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters.

A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg′ normally found in a cell.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, the identity is calculated between two peptides or polynucleotides over their full-length, or over the shorter sequence of the two, or over the longer sequence of the two.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: blast.ncbi.nlm.nih.gov/Blast.cgi, last accessed on Aug. 1, 2021.

In some embodiments, the polynucleotide as disclosed herein is an RNA or an analog thereof. In some embodiments, the polynucleotide as disclosed herein is a DNA or an analog thereof. In some embodiments, the polynucleotide as disclosed herein is a hybrid of DNA and RNA or an analog thereof.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complementary. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” refers to the process whereby a foreign nucleotide sequence is introduced into a cell. In some embodiments, this transduction is done via a vector, viral or non-viral.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, protein or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

A “selection marker” refers to a protein or a gene encoding the protein necessary for survival or growth of a cell grown in a selective culture regimen. Typical selection markers include sequences that encode proteins, which confer resistance to selective agents, such as antibiotics, herbicides, or other toxins. Examples of selection markers include genes for conferring resistance to antibiotics, such as spectinomycin, streptomycin, tetracycline, ampicillin, kanamycin, G 418, neomycin, bleomycin, hygromycin, methotrexate, dicamba, glufosinate, or glyphosate.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

In some embodiments, the cell as disclosed herein is a eukaryotic cell or a prokaryotic cell. In some embodiments, the cell is a human cell. In some embodiments, the cell is a cell line, such as a human embryonic kidney 293 cell (HEK 293 cell or 293 cell), a 293T cell, or an a549 cell. In some embodiments, the cell is a host cell.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The host cell can be a prokaryotic or a eukaryotic cell. In some embodiments, the host cell is a cell line, such as a human embryonic kidney 293 cell (HEK 293 cell or 293 cell), a 293T cell, or an a549 cell. Cultured cells lines are commercially available from the American Type Culture Collection, for example.

As used herein, “Immune cells” includes, e.g., white blood cells (leukocytes, such as granulocytes (neutrophils, eosinophils, and basophils), monocytes, and lymphocytes (T cells, B cells, natural killer (NK) cells and NKT cells)) which may be derived from hematopoietic stem cells (HSC) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSc), hematopoietic stem cells (HSCs), or immortalized cells. In some embodiments, the HSC are derived from umbilical cord blood of a subject, peripheral blood of a subject, or bone marrow of a subject. In some embodiments, the subject from whom the immune cell is directly or indirectly obtained is the same subject to be treated. In some embodiments, the subject from whom the immune cell is directly or indirectly obtained is different from the subject to be treated. In further embodiments, the subject from whom the immune cell is directly or indirectly obtained is different from the subject to be treated and the subjects are from the same species, such as human.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, canine, bovine, porcine, murine, rat, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium. Cultured cells lines are commercially available from the American Type Culture Collection, for example.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include carriers, such as pharmaceutically acceptable carriers. In some embodiments, the carrier (such as the pharmaceutically acceptable carrier) comprises, or consists essentially of, or yet further consists of a nanoparticle, such as an polymeric nanoparticle carrier (for example, an HKP nanoparticle) or an lipid nanoparticle (LNP).

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. In some embodiments, a pharmaceutically acceptable carrier comprises, or consists essentially of, or yet further consists of a nanoparticle, such as an polymeric nanoparticle carrier (for example, an HKP nanoparticle) or an lipid nanoparticle (LNP). Additionally or alternatively, pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination, and can be separately packaged with or without specific dosages. The active ingredients of the combination can be administered concurrently or sequentially.

The four-branched histidine-lysine (HK) peptide polymer H2K4b has been shown to be a good carrier of large molecular weight DNA plasmids (Leng et al. Nucleic Acids Res 2005; 33:e40.), but a poor carrier of relatively low molecular weight siRNA (Leng et al. J Gene Med 2005; 7:977-986.). Two histidine-rich peptides analogs of H2K4b, namely H3K4b and H3K(+H)4b, were shown to be effective carriers of siRNA (Leng et al. J Gene Med 2005; 7: 977-986. Chou et al. Biomaterials 2014; 35:846-855.), although H3K(+H)4b appeared to be modestly more effective (Leng et al. Mol Ther 2012; 20:2282-2290.). Moreover, the H3K4b carrier of siRNA induced cytokines to a significantly greater degree in vitro and in vivo than H3K(+H)4b siRNA polyplexes (Leng et al. Mol Ther 2012; 20:2282-2290.). Suitable HK polypeptides are described in WO/2001/047496, WO/2003/090719, and WO/2006/060182, the contents of each of which are incorporated herein in their entireties. These polypeptides have a lysine backbone (three lysine residues) where the lysine side chain ε-amino groups and the N-terminus are coupled to various HK sequences. HK polypeptide carriers can be synthesized by methods that are well-known in the art including, for example, solid-phase synthesis.

It was found that such histidine-lysine peptide polymers (“HK polymers” or “HKP”) were surprisingly effective as mRNA carriers, and that they can be used, alone or in combination with liposomes, to provide effective delivery of mRNA into target cells. Similar to PEI and other carriers, initial results suggested HK polymers differ in their ability to carry and release nucleic acids. However, because HK polymers can be reproducibly made on a peptide synthesizer, their amino acid sequence can be easily varied, thereby allowing fine control of the binding and release of RNAs, as well as the stability of polyplexes containing the HK polymers and RNA (Chou et al. Biomaterials 2014; 35:846-855. Midoux et al. Bioconjug Chem 1999; 10:406-411. Henig et al. Journal of American Chemical Society 1999; 121:5123-5126.). When mRNA molecules are admixed with one or more HKP carriers the components self-assemble into nanoparticles.

As described herein, advantageously the HK polymer comprises four short peptide branches linked to a three-lysine amino acid core. The peptide branches consist of histidine and lysine amino acids, in different configurations. The general structure of these histidine-lysine peptide polymers (HK polymers) is shown in Formula I, where R represents the peptide branches and K is the amino acid L-lysine.

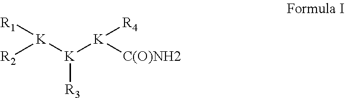

In Formula I where K is L-lysine and each of R₁, R₂, R₃ and R₄ is independently a histidine-lysine peptide. The R₁₋₄ branches may be the same or different in the HK polymers of the invention. When a R branch is “different”, the amino acid sequence of that branch differs from each of the other R branches in the polymer. Suitable R branches used in the HK polymers of the invention shown in Formula I include, but are not limited to, the following R branches R_{A}-R_{-J}:

(SEQ ID NO: 72)
R_{A} = KHKHHKHHKHHKHHKHHKHK-
(SEQ ID NO: 73)
R_{B} = KHHHKHHHKHHHKHHHK-
(SEQ ID NO: 74)
R_{C} = KHHHKHHHKHHHHKHHHK-
(SEQ ID NO: 75)
R_{D} = kHHHkHHHkHHHHHHHk-
(SEQ ID NO: 76)
R_{E} = HKHHHKHHHKHHHHKHHHK-
(SEQ ID NO: 77)
R_{F} = HHKHHHKHHHKHHHHKHHHK-
(SEQ ID NO: 78)
R_{G} = KHHHHKHHHHKHHHHKHHHHK-
(SEQ ID NO: 79)
R_{H} = KHHHKHHHKHHHKHHHHK-
(SEQ ID NO: 80)
R_{I} = KHHHKHHHHKHHHKHHHK-
(SEQ ID NO: 81)
R_{J} = KHHHKHHHHKHHHKHHHHK -

Specific HK polymers that may be used in the mRNA compositions include, but are not limited to, HK polymers where each of R₁, R₂, R₃ and R₄ is the same and selected from R_{A}-R_{J} (Table 1). These HK polymers are termed H2K4b, H3K4b, H3K(+H)4b, H3k(+H)4b, H-H3K(+H)4b, HH-H3K(+H)4b, H4K4b, H3K(1+H)4b, H3K(3+H)4b and H3K(1,3+H)4b, respectively. In each of these 10 examples, upper case “K” represents a L-lysine, and lower case “k” represents D-lysine. Extra histidine residues, in comparison to H3K4b, are underlined within the branch sequences. Nomenclature of the HK polymers is as follows:
1) for H3K4b, the dominant repeating sequence in the branches is -HHHK- (SEQ ID NO: 82), thus “H3K” is part of the name; the “4b” refers to the number of branches;
2) there are four -HHHK- (SEQ ID NO: 82) motifs in each branch of H3K4b and analogues; the first -HHHK- motif (SEQ ID NO: 82) (“1”) is closest to the lysine core;
3) H3K(+H)4b is an analogue of H3K4b in which one extra histidine is inserted in the second -HHHK- motif (SEQ ID NO: 82) (motif 2) of H3K4b;
4) for H3K(1+H)4b and H3K(3+H)4b peptides, there is an extra histidine in the first (motif 1) and third (motif 3) motifs, respectively;
5) for H3K(1,3+H)4b, there are two extra histidines in both the first and the third motifs of the branches.

TABLE 1

| | | Sequence |
|---|---|---|
| Polymer | Branch Sequence | Identifier |
| H2K 4b | R_{A} = KHKHHKHHKHHKHHKHHKHK- | (SEQ ID NO: |
| | | 72) |
| | 4 3 2 1 | |
| H3K4b | R_{B} = KHHHKHHHKHHHKHHHK- | (SEQ ID NO: |
| | | 73) |
| H3K(+H)4b | R_{C} = KHHHKHHHKHHHHKHHHK- | (SEQ ID NO: |
| | | 74) |
| H3k(+H)4b | R_{D} = kHHHkHHHkHHHHkHHHk- | (SEQ ID NO: |
| | | 75) |
| H-H3K(+H)4b | R_{E} = HKHHHKHHHKHHHHKHHHK- | (SEQ ID NO: |
| | | 76) |
| HH-H3K(+H)4b | R_{F} = HHKHHHKHHHKHHHHKHHHK- | (SEQ ID NO: |
| | | 77) |
| H4K4b | R_{G} = KHHHHKHHHHKHHHHKHHHHK- | (SEQ ID NO: |
| | | 78) |
| H3K(1 +H)4b | R_{H} = KHHHKHHHKHHHKHHHHK- | (SEQ ID NO: |
| | | 79) |
| H3K(3 +H)4b | R_{I} = KHHHKHHHHKHHHKHHHK- | (SEQ ID NO: |
| | | 80 |
| H3K(1,3 +H)4b | R_{J} = KHHHKHHHHKHHHKHHHHK- | (SEQ ID NO: |
| | | 81) |

Methods well known in the art, including gel retardation assays, heparin displacement assays and flow cytometry can be performed to assess performance of different formulations containing HK polymer plus liposome in successfully delivering mRNA. Suitable methods are described in, for example, Gujrati et al, Mol. Pharmaceutics 11:2734-2744 (2014), and Parnaste et al., Mol Ther Nucleic Acids. 7: 1-10 (2017).

Detection of mRNA uptake into cells can also be achieved using SMARTFLARE® technology (Millipore Sigma). These smart flares are beads that have a sequence attached that, when recognizing the RNA sequence in the cell, produce an increase in fluorescence that can be analyzed with a fluorescent microscope.

Other methods include measuring protein expressions from an mRNA, for example, an mRNA encoding luciferase can be used to measure the efficiency of transfection. See, for example, He et al (J Gene Med. 2021 February; 23(2):e3295) demonstrating the efficacy of delivering mRNA using a HKP and liposome formulation.

The combination of H3K(+H)4b and DOTAP (a cationic lipid) surprisingly was synergistic in its ability to carry mRNA into MDA-MB-231 cells (H3K(+H)4b/liposomes vs liposomes, P<0.0001). The combination was about 3-fold and 8-fold more effective as carriers of mRNA than the polymer alone and the cationic lipid carrier, respectively. Not all HK peptides demonstrated the synergistic activity with DOTAP lipid. For example, the combination of H3K4b and DOTAP was less effective than the DOTAP liposomes as carriers of luciferase mRNA. Besides DOTAP, other cationic lipids that may be used with HK peptides include Lipofectin (ThermoFisher), Lipofectamine (ThermoFisher), and DOSPER.

The D-isomer of H3k (+H)4b, in which the L-lysines in the branches are replaced with D-lysines, was the most effective polymeric carrier (H3k(+H)4b vs. H3K(+H)4b, P<0.05). The D-isomer/liposome carrier of mRNA was nearly 4-fold and 10-fold more effective than the H3k(+H)4b alone and liposome carrier, respectively. Although the D-H3k(+H)4b/lipid combination was modestly more effective than the L-H3K(+H)4b/lipid combination, this comparison was not statistically different.

Both H3K4b and H3K(+H)4b can be used as carriers of nucleic acids in vitro See, for example, Leng et al. J Gene Med 2005; 7: 977-986; and Chou et al., Cancer Gene Ther 2011; 18: 707-716. Despite these previous findings, H3K(+H)4b was markedly better as a carrier of mRNA compared to other similar analogues (Table 2).

TABLE 2

| | Ratio(wt:wt; | |
|---|---|---|
| Polymer | mRNA:Polymer) | RLU/ug-Protein |
| H3K(+H)4b | 1:4 | 1532.9 ± 122.9 |
| | 1:8 | 1656.3 ± 202.5 |
| | 1:12 | 1033.4 ± 197 |
| H3k(+H)4b | 1:4 | 1851.6 ± 138.3 |
| | 1:8 | 1787.2 ± 195.2 |
| | 1:12 | 1982.3 ± 210.7 |
| H3K 4b | 1:4 | 156.8 ± 41.8 |
| | 1:8 | 62.1 ± 13.2 |
| | 1:12 | 18.1 ± 4.0 |
| H3K(3 + H)4b | 1:4 | 61.7 ± 5.7 |
| | 1:8 | 68.7 ± 3.1 |
| | 1:12 | 59.0 ± 7.5 |
| H3K(1 + H)4b | 1:4 | 24.3 ± 4.5 |
| | 1:8 | 15.0 ± 3.6 |
| | 1:12 | 7.3 ± 2.5 |
| H-H3K(+H)4b | 1:4 | 1107.5 ± 140.4 |
| | 1:8 | 874.6 ± 65.2 |
| | 1:12 | 676.4 ± 25.7 |
| HH-H3K(+H)4b | 1:4 | 1101.9 ± 106.6 |
| | 1:8 | 832.2 ± 75.3 |
| | 1:12 | 739.8 ± 105.4 |
| H4K 4b | 1:4 | 896.4 ± 112.6 |
| | 1:8 | 821.8 ± 115.6 |
| | 1:12 | 522.4 ± 69.2 |
| H3K(1, 3 + H)4b | 1:4 | 518.3 ± 134.7 |
| | 1:8 | 427.7 ± 18.1 |
| | 1:12 | 378 ± 5.2 |
| H2K 4b | 1:4 | 546.7 ± 70.1 |
| | 1:8 | 132.3 ± 58.5 |
| | 1:12 | 194.7 ± 18.4 |

Especially, it has higher mRNA transfection efficiency than H3K4b in various weight:weight (HK:mRNA) ratios. At a 4:1 ratio, luciferase expression was 10-fold higher with H3K(+H)4b than H3K4b in MDA-MB-231 cells without significant cytotoxicity. Moreover, the buffering capacity does not seem to be an essential factor in their transfection differences since the percent of histidines (by weight) in H3K4b and H3K(+H)4b is 68.9 and 70.6%, respectively.

Gel retardation assays show that the electrophoretic mobility of mRNA was delayed by the HK polymers. The retardation effect increased with higher peptide to mRNA weight ratios. However, mRNA was completely retarded in 2:1 ratio of H3K(+H)4b, whereas it was not completely retarded by H3K4b. This suggested that H3K(+H)4b could form a more stable polyplex, which was advantageous for its ability to be a suitable carrier for mRNA delivery.

Further confirmation that the H3K(+H)4b peptide binds more tightly to mRNA was demonstrated with a heparin-displacement assay. Various concentrations of heparin was added into the polyplexes formed with mRNA and HK and it was observed that, particularly at the lower concentrations of heparin, mRNA was released by the H3K4b polymer more readily than the H3K(+H)4b polymer. These data suggest H3K(+H)4b could bind to mRNA and form a more stable polyplex than H3K4b.

With the mRNA labeled with cyanine-5, the uptake of H3K4b and H3K(+H)4b polyplexes into MDA-MB-231 cells was compared using flow cytometry. At different time points (1, 2, and 4 h), the H3K(+H)4b polyplexes were imported into the cells more efficiently than H3K4b polyplexes. Similar to these results, fluorescent microscopy indicated that H3K(+H)4b polyplexes localized within the acidic endosomal vesicles significantly more than H3K4b polyplexes (H3K4b vs. H3K(+H)4b, P<0.001). Interestingly, irregularly-shaped H3K4b polyplexes, which did not overlap endocytic vesicles, were likely extracellular and were not observed with H3K(+H)4b polyplexes.

It is known both that HK polymers and cationic lipids (i.e., DOTAP) significantly and independently increase transfection with plasmids. See, for example, Chen et al. Gene Ther 2000; 7: 1698-1705. Consequently, whether these lipids together with HK polymers enhanced mRNA transfection was investigated. Notably, the H3K(+H)4b and H3k(+H)4b carriers were significantly better carriers of mRNA than the DOTAP liposomes. The combination of H3K(+H)4b and DOTAP lipid was synergistic in the ability to carry mRNA into MDA-MB-231 cells. The combination was about 3-fold and 8-fold more effective as carriers of mRNA than the polymer alone and the liposome carrier, respectively (H3K(+H)4b/lipid vs. liposomes or H3K(+H)4b). Notably, not all HK peptides demonstrated improved activity with DOTAP lipid. The combination of H3K4b and DOTAP carriers was less effective than the DOTAP liposomes as carriers of luciferase mRNA. The combination of DOTAP and H3K(+H)4b carriers were found to be synergistic in their ability to carry mRNA into cells. See, for example, He et al. J Gene Med. 2020 Nov. 10:e3295.

In some embodiments, the carrier, such as the HKP nanoparticle, further comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA, or MC3), and di((Z)-non-2-en-1-yl) 9-((4-(dimethylamino)butanoyl)oxy)heptadecanedioate (L319).

In some embodiments, the carrier is a nanoparticle. As used herein, the term “nanoparticle” refers to any particle having a diameter of less than 1000 nanometers (nm). In some embodiments, the nanoparticles have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g. having diameters of 50 nm or less, e.g., 5 nm-30 nm, are used in some embodiments.

In some embodiments, the carrier is a polymeric nanoparticle. The term “polymeric nanoparticle” refers to a nanoparticle composed of polymer compound (e.g., compound composed of repeated linked units or monomers) including any organic polymers, such as a Histidine-Lysine (HK) polypeptide (HKP).

As used therein, “liposome” refers to one or more lipids forming a complex, usually surrounded by an aqueous solution. Liposomes are generally spherical structures comprising lipids fatty acids, lipid bilayer type structures, unilamellar vesicles and amorphous lipid vesicles. Generally, liposomes are completely closed lipid bilayer membranes containing an entrapped aqueous volume. The liposomes may be unilamellar vesicles (possessing a single bilayer membrane), oligolamellar or multilamellar (an onion-like structure characterized by multiple membrane bilayers, each separated from the next by an aqueous layer).

In some embodiments, the carrier is a lipid nanoparticle (LNP, also referred to herein as a liposomal nanoparticle). In some embodiments, the LNP has a mean diameter of about 50 nm to about 200 nm. In some embodiments, Lipid nanoparticle carriers/formulations typically comprise, or alternatively consist essentially of, or yet further consist of a lipid, in particular, an ionizable cationic lipid, for example, SM-102 as disclosed herein, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319). In some embodiments, the LNP carriers/formulations further comprise a neutral lipid, a sterol (such as a cholesterol) and a molecule capable of reducing particle aggregation, for example a PEG or PEG-modified lipid (also referred to herein as PEGylated lipid). Additional exemplary lipid nanoparticle compositions and methods of making same are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21:1570-1578, the contents of each of which are incorporated herein by reference in their entirety.

In one embodiment, the term “disease” or “disorder” as used herein refers to a cancer, a status of being diagnosed with a cancer, a status of being suspect of having a cancer, or a status of at risk of having a cancer.

As used herein, a “cancer” is a disease state characterized by the presence in a subject of cells demonstrating abnormal uncontrolled replication and in some aspects, the term may be used interchangeably with the term “tumor.” The term “cancer or tumor antigen” or “neoantigen” refers to an antigen known to be associated and expressed in a cancer cell or tumor cell (such as on the cell surface) or tissue, and the term “cancer or tumor targeting antibody” refers to an antibody that targets such an antigen. In some embodiments, the neoantigen does not express in a non-cancer cell or tissue. In some embodiments, the neoantigen expresses in a non-cancer cell or tissue at a level significantly lower compared to a cancer cell or tissue.

In some embodiments, the cancer is selected from: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre- tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. In some embodiments, the cancer is a colon cancer, colorectal cancer or rectal cancer. In some embodiments, the cancer is a lung cancer. In some embodiments, the cancer is a pancreatic cancer. In some embodiments, the cancer is an adenocarcinoma, an adenocarcinoma, an adenoma, a leukemia, a lymphoma, a carcinoma, a melanoma, an angiosarcoma, or a seminoma.

In some embodiments, the cancer is a solid tumor. In other embodiments, the cancer is not a solid tumor. In further embodiments, the cancer is a leukemia cancer. In some embodiments, the cancer is from a carcinoma, a sarcoma, a myeloma, a leukemia, or a lymphoma. In some embodiments, the cancer is a colon cancer, colorectal cancer or rectal cancer. In some embodiments, the cancer is a lung cancer. In some embodiments, the cancer is a pancreatic cancer.

In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

In some embodiments, the cancer expresses a ras mutation as disclosed herein, such as a lung adenocarcinoma, a mucinous adenoma, a ductal carcinoma of the pancreas, a colorectal carcinoma; a rectal cancer, a follicular thyroid cancer, an autoimmune lymphoproliferative syndrome, a Noonan syndrome, a juvenile myelomonocytic leukemia; a bladder cancer, a follicular thyroid cancer, and an oral squamous cell carcinoma. The mutation can be detected by sequencing a biopsy of the cancer, a Southern Blotting, a Northern Blotting, or by contacting with an antibody specifically binding to the mutation, such as Ras (G12D Mutant) Monoclonal Antibody (HL10) available from ThermoFisher, or anti-Ras (mutated G12D) antibody (ab221163) available from abcam.

As used herein, the term “animal” refers to living multi-cellular vertebrate organisms, a category that includes, for example, mammals and birds. The term “mammal” includes both human and non-human mammals such as non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, bat, rat, rabbit, guinea pig).

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, bat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, the subject has or is diagnosed of having a disease. In some embodiments, the subject is suspected of having a disease. In some embodiments, the subject is at risk of having a disease. In some embodiments, the subject is in fully (such as free of cancer) cancer remission. In further embodiments, the subject is at risk of having a recurrence or relapse of a cancer. In some embodiments, the subject is in partially cancer remission. In some embodiments, the subject is at risk of cancer metastasis.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In some embodiments, the terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, a biological sample, or a sample, is obtained from a subject. Exemplary samples include, but are not limited to, cell sample, tissue sample, biopsy, liquid samples such as blood and other liquid samples of biological origin, including, but not limited to, anterior nasal swab, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some embodiments, the biological sample is a tumor biopsy.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

The term “adjuvant” refers to a substance or mixture that enhances the immune response to an antigen. As non-limiting example, the adjuvant can comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a Mycobacterium (See e.g., U.S. Pat. No. 8,241,610). In another embodiment, the synthetic nanocarrier may comprise at least one polynucleotide and an adjuvant. As a non-limiting example, the synthetic nanocarrier comprising and adjuvant can be formulated by the methods described in WO2011150240 and US20110293700, each of which is herein incorporated by reference in its entirety.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

In some embodiments, an RNA, polynucleotide, vector, cell or composition as disclosed herein is administered in an effective amount. An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

In some embodiments, an RNA, polynucleotide, vector, cell or composition as disclosed herein is administered in a therapeutically or pharmaceutically effective amount. “Therapeutically effective amount” or “pharmaceutically effective amount” of an agent refers to an amount of the agent that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. The effect does not necessarily occur by administration of one dose, and may occur only after administration of a series of doses. Thus, a therapeutically or pharmaceutically effective amount may be administered in one or more administrations.

In some embodiments, the treatment method as disclosed herein can be used as a first line treatment, or a second line treatment, or a third line treatment. The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition”. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

An “anti-cancer therapy,” as used herein, includes but is not limited to surgical resection, chemotherapy, cryotherapy, radiation therapy, immunotherapy and targeted therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

In some embodiments where the cancer is an immune cell cancer, an anti-cancer therapy may comprises, or consists essentially of, or consists of a hematopoietic stem cell transplantation.

In some embodiments, a therapeutic agent, such as a cell as disclosed herein, may be combined in treating a cancer with another anti-cancer therapy or a therapy depleting an immune cell. For example, lymphodepletion chemotherapy is performed followed by administration of a cell as disclosed herein, such as four weekly infusions. In further embodiments, these steps may be repeated for once, twice, three or more times until a partial or complete effect is observed or a clinical end point is achieved.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

In some embodiments, the immunotherapy regulates immune checkpoints. In further embodiments, the immunotherapy comprises, or consists essentially of, or yet further consists of an immune checkpoint inhibitor, such as an Cytotoxic T-Lymphocyte Associated Protein 4 (CTLA4) inhibitor, or a Programmed Cell Death 1 (PD-1) inhibitor, or a Programmed Death Ligand 1 (PD-L1) inhibitor. In yet further embodiments, the immune checkpoint inhibitor comprises, or consists essentially of, or yet further consists of an antibody or an equivalent thereof recognizing and binding to an immune checkpoint protein, such as an antibody or an equivalent thereof recognizing and binding to CTLA4 (for example, Yervoy (ipilimumab), CP-675,206 (tremelimumab), AK104 (cadonilimab), or AGEN1884 (zalifrelimab)), or an antibody or an equivalent thereof recognizing and binding to PD-1 (for example, Keytruda (pembrolizumab), Opdivo (nivolumab), Libtayo (cemiplimab), Tyvyt (sintilimab), BGB-A317 (tislelizumab), JS001 (toripalimab), SHR1210 (camrelizumab), GB226 (geptanolimab), JS001 (toripalimab), AB122 (zimberelimab), AK105 (penpulimab), HLX10 (serplulimab), BCD-100 (prolgolimab), AGEN2034 (balstilimab), MGA012 (retifanlimab), AK104 (cadonilimab), HX008 (pucotenlimab), PF-06801591 (sasanlimab), JNJ-63723283 (cetrelimab), MGD013 (tebotelimab), CT-011 (pidilizumab), or Jemperli (dostarlimab)), or an antibody or an equivalent thereof recognizing and binding to PD-L1 (for example, Tecentriq (atezolizumab), Imfinzi (durvalumab), Bavencio (avelumab), CS1001 (sugemalimab), or KN035 (envafolimab)).

As used herein, a “targeted therapy” refers to a cancer therapy using drugs or other substances that block the growth and spread of cancer by interfering with specific molecules (“molecular targets”) that are involved in the growth, progression, relapse, and spread of cancer, such as T cells or NK cells or other immune cells expressing a chimeric antigen receptor (CAR) which specifically targets and binds a neoantigen. In some embodiments, the neoantigen targeted by this targeted therapy can be the same with one encoded by an RNA as disclosed herein. In other embodiments, the neoantigen targeted by this targeted therapy is different from those encoded by an RNA as disclosed herein.

As used herein, a cleavable peptide, which is also referred to as a cleavable linker, means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving (SEQ ID NO: 99).

In some embodiments, the term “linker” refers to any amino acid sequence comprising from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (G4S)n, wherein n is 1, or 2, or 3, or 4, or 5, or 6, or 7, or 8, or 9, or 10, or 11, or 12, or 13, or 14, or 15 (SEQ ID NO: 100).

As used herein, the phrase “derived” means isolated, purified, mutated, or engineered, or any combination thereof. For example, a ras derived peptide refers to a peptide engineered from a ras gene or a RAS protein, such as a wild-type one. In some embodiments, a ras derived peptide is a RAS mutant, or a fragment thereof.

In some embodiments, a “signal peptide” refers to a peptide sequence that directs the transport and localization of the protein within a cell, e.g. to a certain cell organelle (such as the endoplasmic reticulum) and/or the cell surface and/or secreted outside of the cell. In some embodiments, the signal peptide is at the N terminus of the protein and can be cleaved to produce the mature protein. In some embodiments, the signal peptide is about 15 to about 30 amino acid long.

As used herein, an open reading frame (ORF) refers to a sequence of nucleotides that encodes a polypeptide or a portion thereof. In some embodiments, the ORF is an RNA.

As used herein, a mutation refers to an insertion, a substitution, a deletion, a missense mutation, or a combination thereof. In some embodiments, the terms “mutation” and “mutant” are used interchangeably. In some embodiments, a mutant refers to a mutated polypeptide, or polynucleotide, or a fragment thereof.

As used herein, the term “ras” refers to A family of genes that make proteins involved in cell signaling pathways that control cell growth and cell death. Mutated forms of the ras gene can be found in some types of cancer. These changes may cause cancer cells to grow and spread in the body. Members of the ras gene family include kras (also referred to herein as k-ras), hras (also referred to herein as h-ras), and nras (also referred to herein as n-ras). In some embodiments, the gene name in non-capitalized letters also refer to the encoded protein. In other embodiments, the capitalized name, such as RAS, KRAS, NRAS, refers to the encoded protein.

As used herein, the terms “kras,” and “k-ras” refer to Kirsten Rat Sarcoma Viral Proto-Oncogene, or a protein encoded thereby. This gene encodes a protein that is a member of the small GTPase superfamily. A single amino acid substitution is responsible for an activating mutation. The transforming protein that results is implicated in various malignancies, including lung adenocarcinoma, mucinous adenoma, ductal carcinoma of the pancreas and colorectal carcinoma. Non-limiting exemplary sequences of this protein or the underlying gene may be found under Gene Cards ID: GC12M025204 (retrieved from www.genecards.org/cgi-bin/carddisp.pl?gene=KRAS, last accessed on Oct. 9, 2021), HGNC: 6407 (retrieved from www.genenames.org/data/gene-symbol-report/#!/hgnc_id/6407, last accessed on Oct. 9, 2021), NCBI Entrez Gene: 3845 (retrieved from www.ncbi.nlm.nih.gov/gene/3845, last accessed on Oct. 9, 2021), Ensembl: ENSG00000133703 (retrieved from useast.ensembl.org/Homo_sapiens/Gene/Summary?g=ENSG00000133703; r=12:2520 5246-25250936, last accessed on Oct. 9, 2021), OMIM®: 190070 (retrieved from omim.org/entry/190070, last accessed on Oct. 9, 2021), or UniProtKB/Swiss-Prot: P01116 (retrieved from www.uniprot.org/uniprot/P01116, last accessed on Oct. 9, 2021), which are incorporated by reference herein.

In some embodiments, a KRAS protein is a wild-type KRAS protein (such as of a healthy subject or a subject free of a cancer) that comprises, or consists essentially of, or yet further consists of

(SEQ ID NO: 101)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQRVEDAFYTLVREIRQYRLKKISKEEKTPGCVKIKKCIIM
or
(SEQ ID NO: 102)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHHYRE
QIKRVKDSEDVPMVLVGNKCDLPSRTVDTKQAQDLARSYGIPFIETSAK
TRQGVDDAFYTLVREIRKHKEKMSKDGKKKKKKSKTKCVIM

As used herein, the terms “nras,” and “n-ras” refer to Neuroblastoma RAS Viral Oncogene Homolog, or a protein encoded thereby. This is an N-ras oncogene encoding a membrane protein that shuttles between the Golgi apparatus and the plasma membrane. This shuttling is regulated through palmitoylation and depalmitoylation by the ZDHHC9-GOLGA7 complex. The encoded protein, which has intrinsic GTPase activity, is activated by a guanine nucleotide-exchange factor and inactivated by a GTPase activating protein. Mutations in this gene have been associated with somatic rectal cancer, follicular thyroid cancer, autoimmune lymphoproliferative syndrome, Noonan syndrome, and juvenile myelomonocytic leukemia. Non-limiting exemplary sequences of this protein or the underlying gene may be found under Gene Cards ID: GC01M114704 (retrieved from www.genecards.org/cgi-bin/carddisp.pl?gene=NRAS, last accessed on Oct. 9, 2021), HGNC: 7989 (retrieved from www.genenames.org/data/gene-symbol-report/#!/hgnc_id/7989, last accessed on Oct. 9, 2021), NCBI Entrez Gene: 4893 (retrieved from www.ncbi.nlm.nih.gov/gene/4893, last accessed on Oct. 9, 2021), Ensembl: ENSG00000213281 (retrieved from useast.ensembl.org/Homo_sapiens/Gene/Summary?g=ENSG00000133703; r=12:2520 5246-25250936, last accessed on Oct. 9, 2021), OMIM®: 164790 (retrieved from omim.org/entry/164790, last accessed on Oct. 9, 2021), or UniProtKB/Swiss-Prot: P01111 (retrieved from www.uniprot.org/uniprot/P01111, last accessed on Oct. 9, 2021), which are incorporated by reference herein.

In some embodiments, a NRAS protein is a wild-type NRAS protein (such as of a healthy subject or a subject free of a cancer) that comprises, or consists essentially of, or yet further consists of

(SEQ ID NO: 103)
MTEYKLVVVGAGGVGKSALTIQLIQNHFVDEYDPTIEDSYRKQVVIDGE
TCLLDILDTAGQEEYSAMRDQYMRTGEGFLCVFAINNSKSFADINLYRE
QIKRVKDSDDVPMVLVGNKCDLPTRTVDTKQAHELAKSYGIPFIETSAK
TRQGVEDAFYTLVREIRQYRMKKLNSSDDGTQGCMGLPCVVM

As used herein, the terms “hras,” and “h-ras” refer to Harvey Rat Sarcoma Viral Oncogene Homolog, or a protein encoded thereby. The products encoded by these genes function in signal transduction pathways. These proteins can bind GTP and GDP, and they have intrinsic GTPase activity. This protein undergoes a continuous cycle of de- and re-palmitoylation, which regulates its rapid exchange between the plasma membrane and the Golgi apparatus. Mutations in this gene cause Costello syndrome, a disease characterized by increased growth at the prenatal stage, growth deficiency at the postnatal stage, predisposition to tumor formation, cognitive disability, skin and musculoskeletal abnormalities, distinctive facial appearance and cardiovascular abnormalities. Defects in this gene are implicated in a variety of cancers, including bladder cancer, follicular thyroid cancer, and oral squamous cell carcinoma. Non-limiting exemplary sequences of this protein or the underlying gene may be found under Gene Cards ID: GC11M001525 (retrieved from www.genecards.org/cgi-bin/carddisp.pl?gene=HRAS, last accessed on Oct. 9, 2021), HGNC: 5173 (retrieved from www.genenames.org/data/gene-symbol-report/#!/hgnc_id/5173, last accessed on Oct. 9, 2021), NCBI Entrez Gene: 3265 (retrieved from www.ncbi.nlm.nih.gov/gene/3265, last accessed on Oct. 9, 2021), Ensembl: ENSG00000174775 (retrieved from useast.ensembl.org/Homo_sapiens/Gene/Summary?g=ENSG00000174775; r=11:5322 42-537321, last accessed on Oct. 9, 2021), OMIM®: 190020 (retrieved from omim.org/entry/190020, last accessed on Oct. 9, 2021), or UniProtKB/Swiss-Prot: P01112 (retrieved from www.uniprot.org/uniprot/P01112, last accessed on Oct. 9, 2021), which are incorporated by reference herein.

In some embodiments, a HRAS protein is a wild-type HRAS protein (such as of a healthy subject or a subject free of a cancer) that comprises, or consists essentially of, or yet further consists of

(SEQ ID NO: 104)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHQYRE
QIKRVKDSDDVPMVLVGNKCDLAARTVESRQAQDLARSYGIPYIETSAK
TRQGVEDAFYTLVREIRQHKLRKLNPPDESGPGCMSCKCVLS
or
(SEQ ID NO: 105)
MTEYKLVVVGAGGVGKSALTIQLIQNHFVDEYDPTIEDSYRKQVVIDGE
TCLLDILDTAGQEEYSAMRDQYMRTGEGFLCVFAINNTKSFEDIHQYRE
QIKRVKDSDDVPMVLVGNKCDLAARTVESRQAQDLARSYGIPYIETSAK
TRQGSRSGSSSSSGTLWDPPGPM

Of all genes that their mutations lead to cancer, the ras family is one of the earliest identified and also one of the most common. Ras genes were discovered more than 30 years ago. They encode a family of proteins with 188 amino acid residues that has GTPase activity and plays an essential role in cellular signal transduction pathways that regulate a wide range of normal cellular functions which include controlling cell growth and death. However, mutations of three human ras genes, Kirsten rat sarcoma viral oncogene homolog (kras), neuroblastoma RAS viral oncogene homolog (nras) and Harvey rat sarcoma viral oncogene homolog (hras), had been shown as a driving force in human cancers. Among these three genes, kras alone was believed to be involved in approximately one third of human cancer cases. In fact, ras gene mutation is one of the most common driver mutations in the top three most deadly cancers, lung, colorectal and pancreatic cancers.

Targeting gain-of-function mutations of ras had been proposed long ago as a potential effective cancer treatment. However, despite a significant amount of research and development effort invested in this field, ras specific inhibitors, either small molecules or biologics that block the function of mutated versions of RAS proteins had not been successfully developed.

Developing a ras inhibitor faces tremendous technical challenge due to the location, function and structure of the RAS protein. First, as an intracellular protein, RAS is inaccessible for many biologics and small molecules. Second, RAS is a globular protein, and does not have large cervices or grooves on its surface that a small molecule inhibitor can bind effectively. Finally, normal ras is a house keeping gene that plays an important role in maintaining essential cellular functions. Shutting down the activity of the mutated RAS protein only without unwanted impact on normal RAS is extremely challenging. Often, there is only single amino acid change in RAS mutants. Targeted inhibition of the sequence difference between normal and mutant RAS in such a miniature scale continues to be a goal of ras based cancer drug development.

### MODES FOR CARRYING OUT THE DISCLOSURE

Cancer treatment modalities traditionally include surgery, chemotherapy, and radiation therapy. More recently, with in-depth knowledge gained about the molecular pathology of cancer, targeted therapy and immunotherapy had been developed. Both have demonstrated promising results in cancer management. Cancer targeted therapy utilizes sequence information to inhibit the activities of protein products of cancer driver mutations. Because most somatic mutations extend beyond single anatomical sites or cancer types, targeted therapy can be applied to different tumors that share the same underlying mutations regardless their tissue locations. Since 2017, the U.S. Food and Drug Administration (FDA) has approved several treatments for specific genetic defect regardless tissue distribution. Examples include pembrolizumab that is approved for patients with unresectable or metastatic, microsatellite instability-high (MSI-H) or mismatch repair deficient (dMMR) solid tumors and entrectinib for patients with NTRK (neurotrophic tyrosine receptor kinase) gene fusion.

Like targeted therapy, cancer immunotherapy also has potential to treat more than one type of cancer. Cancer immunotherapy utilizes a patient's immune system to fight against tumor cells. Some cancer immunotherapies primarily focus on humoral components of immune systems, the antibodies, to kill cancer cells by inhibiting the function of proteins expressed by cancer cells. Other cancer immunotherapies exert its function through cytotoxic T cells that have the ability to destroy tumor cells directly. The human immune system, as part of its normal functions, surveils and kills abnormal cells by recognizing mutated gene products that do not appear in normal cells and, thus, prevents or curbs the growth of cancers. The mutated version of proteins produced by cancer cells are often called tumor associated antigens, also known as neoantigens. By exposing the immune system to cancer neoantigens, it is possible to enhance the human immune system's ability to target and kill tumor cells. This modality is called cancer treatment vaccine. Human tumor cell lysates or purified tumor neoantigens can be used to stimulate tumor specific immune response from cancer patients. Many different cell components of the immune system can be used to produce a cancer vaccine. As the first FDA approved cancer treatment vaccine, a fusion protein that is consisted of a tumor neoantigen, prostatic acid phosphatase and an adjuvant, granulocyte-macrophage colony-stimulating factor, was loaded into patient's own dendritic cells. Dendritic cells function as the primary antigen-presenting cells (APC) that are responsible for displaying the neoantigens to be recognized by cytotoxic cells. Other cells can also function as APCs.

Despite the great promise of cancer treatment vaccines, there are a great number of technical challenges from immune-epitope discovery to vaccine manufacturing. RNA based vaccines are proposed as a possible solution to the challenges and have shown promise in preclinical and clinical studies. A key advantage of mRNA vaccines is that mRNA can be produced in the laboratory from a DNA template using readily available materials, less expensively and faster than conventional vaccine production, which can require the use of chicken eggs or other mammalian cells. In addition, mRNA vaccines have the potential to streamline vaccine discovery and development, and facilitate a rapid response to emerging infectious diseases (see, for example, Maruggi et al., Mol Ther. 2019; 27(4): 757-772).

During the last two decades, there has been broad interest in RNA-based technologies for the development of prophylactic and therapeutic vaccines. In this field, mRNA vaccines have been investigated extensively for infectious disease prevention, and for cancer prophylaxis and treatment. Preclinical and clinical trials have shown that mRNA vaccines provide a safe and long-lasting immune response in animal models and humans. mRNA vaccines expressing antigens of infectious pathogens induce potent T cell and humoral immune responses (Pardi et al. Nat Rev Drug Discov. 2018; 17: 261-279). As previously described, the production procedure to generate mRNA vaccines is entirely cell-free, simple, and rapid, if compared to production of whole microbe, live attenuated, and subunit vaccines. This fast and simple manufacturing process makes mRNA a promising bio-product that can potentially fill the gap between emerging infectious disease and the desperate need for effective vaccines.

Compared with traditional plasmid and viral-based approaches, this approach allows design of patient-personalized mRNAs that also benefit from eliminating needing to pass through the nuclear membrane (unlike DNA) and thus carries little to no risk of genomic integration. Furthermore, mRNA vaccines are safe, simple, and inexpensive and possess maximum flexibility. Particularly compared with peptide vaccines, they have self-adjuvanting properties, lack of MHC haplotype restriction, and do not need to enter the nucleus (Schlake et al., RNA Biol. 2012; 9(11): 1319-1330]. mRNA does not integrate into the genome and therefore it avoids oncogenesis and mutagenesis (McNamara et al., J Immunol Res. 2015; 2015:794528]. These vaccines are temporary information carriers due to early metabolic degradation within a few days. Last but not least is that any protein can be encoded for development of therapeutic and prophylactic vaccines, without affecting the properties of the mRNA.

Recently, self-amplifying mRNA vaccines have been proved to be safe and effective against human viral pathogens (e.g. influenza). Influenza mRNA vaccines hold great promises, being an egg-free platform and leading to production of antigen with high fidelity in mammalian cells. Recent published results demonstrated that the loss of a glycosylation site by a mutation in the hemagglutinin (HA) of the egg-adapted H3N2 vaccine strain resulted in poor neutralization of circulating H3N2 viruses in vaccinated humans and ferrets (Zost et al., Proc Natl Acad Sci USA. 2017; 114: 12578-12583). By contrast, the process of mRNA vaccine production is egg-free, and mRNA-encoded proteins are properly folded and glycosylated in host cells after vaccine administration, thus avoiding the risk of producing incorrect antigens.

Generation of a robust immune response in infants and the elderly has always been an issue for influenza vaccines. However, mRNA vaccines may benefit in that they have been demonstrated to induce balanced, long-lived and protective immunity to influenza A virus infections in even very young and very old mice. Vaccines based on mRNA or RNA replicons have also been shown to be immunogenic in a variety of animal models, including nonhuman primates (Maruggi et al., Vaccine. 2017; 35(2):361-368).

Target Selection of Pan-Ras mRNA Vaccine

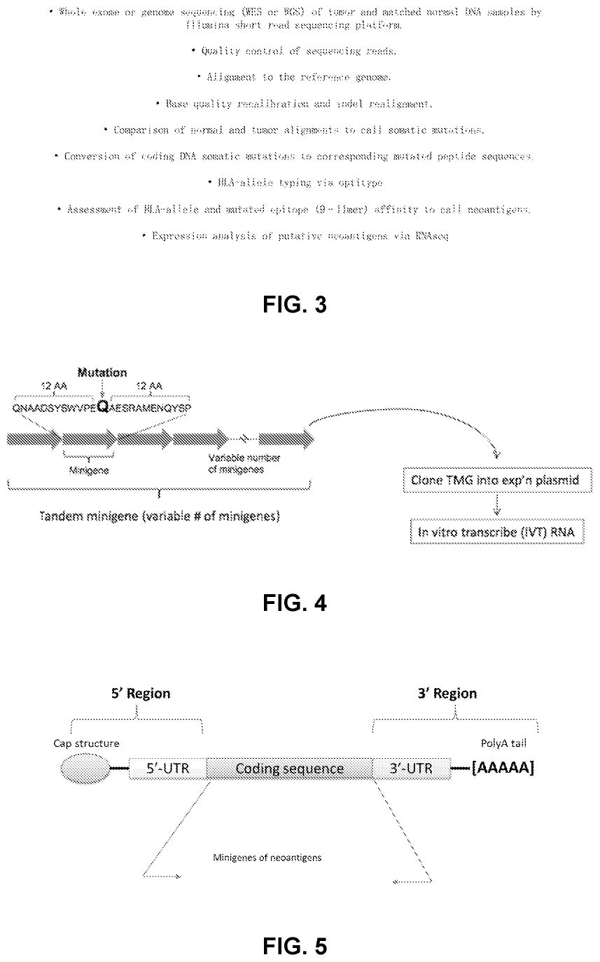

Due to the prominent role ras genes play in cancer molecule pathology, a great amount of preclinical and clinical research had been conducted to investigate the relationship between specific ras mutations, the activity of the mutated RAS proteins and subsequent tumor cell transformation in many different types of cancers. When large scale genomic sequencing technology became available in characterizing somatic mutations of cancer cells, the nature of ras gene somatic mutations was extensively profiled. In the Cancer Genome Atlas Project (TCGA), the largest and most comprehensive effort to date to characterize the genetic changes that drive human cancers, the mutation landscape of ras genes was documented in more than 30 cancer types with over 10,000 tumor samples. Using combination of different sequencing technologies include exome or whole genome sequencing, as well as RNAseq (for transcription and miRNAs) and methylation profiling (for epigenetic correlations), the frequency and tissue type of different somatic ras mutations had been recorded. The mutation sequence frequency established a basis of selecting potential neoantigen epitope for mRNA based ras vaccines. Specifically, next generation sequencing technologies were used to compare both tumor and matched normal samples' sequence data to identify neoantigens. Ras mutations such as single nucleotide variations (SNV) and insertions/deletions had been characterized with statistics software and then validated for the capability in stimulating CD4 and CD8 T cell responses. This RAS candidate neoantigen prediction process involves multiple steps, including somatic mutation identification, HLA typing, peptide processing, and peptide-MHC binding prediction. The selected SNVs were subjected to selection using HLA binding prediction algorithms to screen and identify candidate peptide sequences with strong HLA binding affinity. These peptides are envisioned to have best chance to elicit strong effector T cell inside human body. The peptide candidates that had been predicted by computational methods were then get validated using cancer patients derived peripheral blood monocytes (PBMC) to measure their ability to induce strong in vitro T cell activity. The sequence of the neoantigen peptide candidates with proven in vitro activity was used in mRNA expression construct. Overall, the general workflow has been illustrated in the chart of FIG. 3, including next generation sequencing reads alignment, bam file processing, somatic calling, false positive filtering, neoantigen prediction, HLA typing, HLA binding, and neoantigen prioritization, delivery, and validation. Details of selection and validation neoantigen is further elucidated in FIG. 3.

Construct of Pan-Ras mRNA Vaccine and its Expression Vector

A single mRNA molecule can be engineered to express a polypeptide that had been selected as described herein and be delivered into human cells. The expressed mutated ras peptide can be processed and presented on the surface of APCs and elicit cytotoxic T cells to target destroy cells expressing mutant ras proteins such as tumor cells. Furthermore, because the size of the epitope to be presented by APCs is normally 20-27 amino acid residual long, a single RNA expression construct has the capacity of expressing multiple ras mutant peptides of different sequences. Therefore, several ras mutation peptides can be packed into a single RNA expression product which has the ability to elicit effector T cells towards more than one type of ras mutations. Thus, a pan ras RNA vaccine can be produced in such manner.

Specifically, in some embodiments, each ras neoantigen (also referred to herein as an immunogenic fragment of ras or a ras derived peptide) has 25 amino acid residues with the mutated amino acid residue occupying the 13th position of the ras neoantigen. Multiple ras neoantigens with different mutation sequences can be arranged in tandem separated by non-immunogenic glycine/serine linkers (start linker LQ for P01-P07 or GGSGGGGSGG, SEQ ID NO: 83; middle linker GGSGGGGSGG, SEQ ID NO: 84; and end linker GGSLGGGGSG, SEQ ID NO: 85). Synthetic DNA fragments that encode multiple ras neoantigens in configuration of “tandem minigene” was inserted into an mRNA expression vector. The detailed peptide sequence of producing such pan-ras vaccine is disclosed herein.

As described herein, high frequency somatic ras mutation sequences were identified, and, based on that, polypeptide sequence most likely to induce clinically significant effector T cell activity against ras mutation driven cancer cells were determined. Several pan kras vaccines have been developed, using an immunogenic composition that comprises, or consists essentially of, or further consists of a messenger ribonucleic acid (mRNA) comprising, or consisting essentially of, or yet further consisting of an open reading frame (ORF) encoding one or multiple peptides of different ras mutations, formulated in a pharmaceutically acceptable carrier. In some embodiments, the pharmaceutically acceptable carrier comprises, or consists essentially of, or yet further consists of a polymeric nanoparticle or a liposomal nanoparticle or both. The composition can be administered to a subject in an amount effective to induce a specific immune response against ras neoantigens in such subject.

Accordingly, in one aspect, provided is an isolated ribonucleic acid (RNA) comprising, or consisting essentially of, or yet further consisting of an open reading frame (ORF) encoding a ras derived peptide. In some embodiments, the RNA is formulated in a carrier, such as a pharmaceutically carrier. In further embodiments, the RNA is encapsulated in a nanoparticle. In some embodiments, the encoded ras derived peptide comprises any one or more (such as any one, or any two, or any three, or any four, or all five) of the following mutations:
a mutated residue, such as a phenylalanine (F), aligned to the 19^{th} amino acid residue of SEQ ID NO: 70 (referred to herein as L19F);
a mutated residue, such as a threonine (T), a glycine (G), a glutamic acid (E) or a serine (S), aligned to the 59^{th} amino acid residue of SEQ ID NO: 70 (referred to herein as A59T, A59G, A59E, or A59S respectively);
a mutated residue, such as an aspartic acid (D), a glutamic acid (E), a valine (V), or an arginine (R), aligned to the 60^{th} amino acid residue of SEQ ID NO: 70 (referred to herein as G60D, G60E, G60V, or G60R, respectively);
a mutated residue, such as an asparagine (N) or an R, aligned to the 117^{th} amino acid residue of SEQ ID NO: 70 (referred to herein as K117N or K117R respectively); or
a mutated residue, such as a T, a V, or a proline (P), aligned to the 146^{th} amino acid residue of SEQ ID NO: 70 (referred to herein as A146T, A146V, or A146P, respectively).

In some embodiments, the encoded ras derived peptide further comprises any one or more (such as any one, or any two, or all three) of the following mutations:
a mutated residue, such as a D, an alanine (A), a cysteine (C), an R, an S, or a V, aligned to the 12^{th} amino acid residue of SEQ ID NO: 70 (referred to herein as G12D, G12A, G12C, G12R, G12S, or G12V, respectively);
a mutated residue, such as a D, an A, a C, an R, an S, or a V, aligned to the 13^{th} amino acid residue of SEQ ID NO: 70 (referred to herein as G13D, G13A, G13C, G13R, G13S, or G13V, respectively); or
a mutated residue, such as a histidine (H), an E, a lysine (K), a leucine (L), a P, or an R, aligned to the 61^{th} amino acid residue of SEQ ID NO: 70 (referred to herein as Q61H, Q61E, Q61K, Q61L, Q61P, or Q61R, respectively).

In some embodiments, the encoded ras derived peptide comprises the following mutations: D aligned to the 12^{th} amino acid residue of SEQ ID NO: 70 (G12D), D aligned to the 13^{th} amino acid residue of SEQ ID NO: 70 (G13D); F aligned to the 19^{th} amino acid residue of SEQ ID NO: 70 (L19F); T aligned to the 59^{th} amino acid residue of SEQ ID NO: 70 (A59T); D aligned to the 60^{th} amino acid residue of SEQ ID NO: 70 (G60D); H aligned to the 61^{th} amino acid residue of SEQ ID NO: 70 (Q61H); N aligned to the 117^{th} amino acid residue of SEQ ID NO: 70 (K117N); or T aligned to the 146^{th} amino acid residue of SEQ ID NO: 70 (A146T).

In some embodiments, the ras derived peptide comprises, or consists essentially of, or yet further consist of the polypeptide as set forth in SEQ ID NO: 70, or an equivalent thereof. In some embodiment, the equivalent to SEQ ID NO: 70 retains the following mutations: D aligned to the 12^{th} amino acid residue of SEQ ID NO: 70 (G12D), D aligned to the 13^{th} amino acid residue of SEQ ID NO: 70 (G13D); F aligned to the 19^{th} amino acid residue of SEQ ID NO: 70 (L19F); T aligned to the 59^{th} amino acid residue of SEQ ID NO: 70 (A59T); D aligned to the 60^{th} amino acid residue of SEQ ID NO: 70 (G60D); H aligned to the 61^{th} amino acid residue of SEQ ID NO: 70 (Q61H); N aligned to the 117^{th} amino acid residue of SEQ ID NO: 70 (K117N); and T aligned to the 146^{th} amino acid residue of SEQ ID NO: 70 (A146T).

In one embodiment, the composition comprises, or consists essentially of, or yet further consists of one mRNA encoding eight different kras high-frequency mutation peptides. In some embodiments, each of the peptides comprises a mutation of the following: a mutated residue, such as a phenylalanine (F), aligned to the 19^{th} amino acid residue of SEQ ID NO: 70 (referred to herein as L19F); a mutated residue, such as a threonine (T), a glycine (G), a glutamic acid (E) or a serine (S), aligned to the 59^{th} amino acid residue of SEQ ID NO: 70 (referred to herein as A59T, A59G, A59E, or A59S respectively); a mutated residue, such as an aspartic acid (D), a glutamic acid (E), a valine (V), or an arginine (R), aligned to the 60^{th} amino acid residue of SEQ ID NO: 70 (referred to herein as G60D, G60E, G60V, or G60R, respectively); a mutated residue, such as an asparagine (N) or an R, aligned to the 117^{th} amino acid residue of SEQ ID NO: 70 (referred to herein as K117N or K117R respectively); a mutated residue, such as a T, a V, or a proline (P), aligned to the 146^{th} amino acid residue of SEQ ID NO: 70 (referred to herein as A146T, A146V, or A146P, respectively); a mutated residue, such as a D, an alanine (A), a cysteine (C), an R, an S, or a V, aligned to the 12^{th} amino acid residue of SEQ ID NO: 70 (referred to herein as G12D, G12A, G12C, G12R, G12S, or G12V, respectively); a mutated residue, such as a D, an A, a C, an R, an S, or a V, aligned to the 13^{th} amino acid residue of SEQ ID NO: 70 (referred to herein as G13D, G13A, G13C, G13R, G13S, or G13V, respectively); or a mutated residue, such as a histidine (H), an E, a lysine (K), a leucine (L), a P, or an R, aligned to the 61^{th} amino acid residue of SEQ ID NO: 70 (referred to herein as Q61H, Q61E, Q61K, Q61L, Q61P, or Q61R, respectively). In some embodiments, each of the peptides comprises a mutation of the following: D aligned to the 12^{th} amino acid residue of SEQ ID NO: 70 (G12D), D aligned to the 13^{th} amino acid residue of SEQ ID NO: 70 (G13D); F aligned to the 19^{th} amino acid residue of SEQ ID NO: 70 (L19F); T aligned to the 59^{th} amino acid residue of SEQ ID NO: 70 (A59T); D aligned to the 60^{th} amino acid residue of SEQ ID NO: 70 (G60D); H aligned to the 61^{th} amino acid residue of SEQ ID NO: 70 (Q61H); N aligned to the 117^{th} amino acid residue of SEQ ID NO: 70 (K117N); or T aligned to the 146^{th} amino acid residue of SEQ ID NO: 70 (A146T). In further embodiments, the peptides are different with each other, i.e., comprise different mutations. On the basis of the cDNA clones, the BepiPred linear epitope prediction algorithm was used to select 8 short peptide fragments that can be potential epitopes as targets. The short peptide selected is 25 amino acid residual long with the mutated amino acid residual occupies the central position (amino acid residue 13). Based on these short peptide sequences, corresponding mRNA sequences were designed.

In another embodiment, provided is an mRNA sequence that encodes one, or two, or three, or four, or five, or six, or seven, or eight hras derived peptides. In some embodiments, the mRNA encodes four derived peptides and the peptides comprises the following four mutations of:
a mutated residue, such as a D, an A, a C, an R, an S, or a V, aligned to the 12^{th} amino acid residue of SEQ ID NO: 70 (referred to herein as G12D, G12A, G12C, G12R, G12S, or G12V, respectively);
a mutated residue, such as a D, a C, an R, an S, or a V, aligned to the 13^{th} amino acid residue of SEQ ID NO: 70 (referred to herein as G13D, G13C, G13R, G13S, or G13V, respectively);
a mutated residue, such as an H, a K, an L, a P, or an R, aligned to the 61^{th} amino acid residue of SEQ ID NO: 70 (referred to herein as Q61H, Q61K, Q61L, Q61P, or Q61R, respectively); and
a mutated residue, such as an N, aligned to the 117^{th} amino acid residue of SEQ ID NO: 70 (referred to herein as K117N).

In another embodiment, provided is an mRNA sequence that encodes one, or two, or three, or four, or five, or six, or seven, or eight nras derived peptides. In some embodiments, the mRNA encodes four derived peptides and the peptides comprises the following four mutations of:
a mutated residue, such as a D, an A, a C, an R, an S, or a V, aligned to the 12^{th} amino acid residue of SEQ ID NO: 70 (referred to herein as G12D, G12A, G12C, G12R, G12S, or G12V, respectively);
a mutated residue, such as a D, an A, a C, an R, an S, or a V, aligned to the 13^{th} amino acid residue of SEQ ID NO: 70 (referred to herein as G13D, G13A, G13C, G13R, G13S, or G13V, respectively);
a mutated residue, such as a D, a C, an R, an S, or a V, aligned to the 13^{th} amino acid residue of SEQ ID NO: 70 (referred to herein as G13D, G13C, G13R, G13S, or G13V, respectively);
a mutated residue, such as an E, a V, or an R, aligned to the 60^{th} amino acid residue of SEQ ID NO: 70 (referred to herein as G60E, G60V, or G60R, respectively); and
a mutated residue, such as an H, an E, a K, an L, a P, or an R, aligned to the 61^{th} amino acid residue of SEQ ID NO: 70 (referred to herein as Q61H, Q61E, Q61K, Q61L, Q61P, or Q61R, respectively).

In the description herein, SEQ ID NO: 70 has been used as a reference sequence when identifying a ras mutation. However, one of skill in the art can align the sequence as set forth in SEQ ID NO: 70 with another ras polypeptide and use the other ras polypeptide as a reference sequence to identifying a ras mutation as disclosed herein. For example, an alignment among the sequences set forth in SEQ ID NOs: 70, 101, 103 and 104 was performed with the default setting using Clustal Omega accessible at www.ebi.ac.uk/Tools/msa/clustalo/. The result is provided in FIG. 14. The sequences were aligned with each other from the 1st amino acid residue to the 175th one. Accordingly, the mutations as disclosed herein can be identified by referring to SEQ ID NO: 70, or alternatively by any one of SEQ ID NOs: 101, 103 or 104, without changing the amino acid number as specified.

In another embodiment, provided is an mRNA sequence that encodes sixteen peptides that correspond to, eight kras mutations, four hras mutations, and four different nras mutations.

Besides traditional mRNA-based vaccines, self-amplifying mRNA (SAM) vaccines have been developed. The SAM vaccine uses the host cell's transcription system to produce target antigens to stimulate adaptive immunity. The SAM vaccine encodes the same sets of neoantigens. The SAM vaccine can express antigen at a high level.

With appropriate modification and optimization, as well as well-defined delivery carriers and administration route, pan-ras mRNA vaccines demonstrate improved stability, increased translation efficiency, and enhanced immunogenicity in both mouse and non-human primates (NHP) models.

In one aspect, provided is a ribonucleic acid (RNA) comprising, or consisting essentially of, or yet further consisting of an open reading frame (ORF) encoding one or more ras derived peptides. In some embodiments, each of the one or more ras derived peptides consists of between 23 and 29 amino acid residues. In further embodiments, each of the one or more ras derived peptides consists of about 25 amino acid residues. In some embodiments, the encoded peptides are selected from the group as set forth in SEQ ID NOs:1-69, or an equivalent of each thereof. In some embodiments, the ras derived peptides are selected from kras derived peptides, for example those as set forth in SEQ ID NOs:1-31, or an equivalent of each thereof. In some embodiments, the ras derived peptides are selected from nras derived peptides, for example those as set forth in SEQ ID NOs:32-52, or an equivalent of each thereof. In some embodiments, the ras derived peptides are selected from hras derived peptides, for example those as set forth in SEQ ID NOs: 53-69, or an equivalent of each thereof. In some embodiments, the ras derived peptides do not comprise any one or more of SEQ ID NOs: 1-18, 32-49 or 53-68. Additionally or alternatively, the ras derived peptides are selected from the group as set forth in SEQ ID NOs: 19-31, 50-52 or 69. In some embodiments, the equivalent of any one of SEQ ID NOs: 1-69 retains the mutation of the one of SEQ ID NOs: 1-69.

Peptide sequences of ras neoantigens:
KRAS
SEQ ID NO: 1 G12C:
mteyklvvvgacgvgksaltiqliq
SEQ ID NO: 2 G12A:
mteyklvvvgaagvgksaltiqliq
SEQ ID NO: 3 G12D:
mteyklvvvgadgvgksaltiqliq
SEQ ID NO: 4 G12R:
mteyklvvvgargvgksaltiqliq
SEQ ID NO: 5 G12S:
mteyklvvvgasgvgksaltiqliq
SEQ ID NO: 6 G12V:
mteyklvvvgavgvgksaltiqliq
SEQ ID NO: 7 G13C:
mteyklvvvgagcvgksaltiqliq
SEQ ID NO: 8 G13A:
mteyklvvvgagavgksaltiqliq
SEQ ID NO: 9 G13D:
mteyklvvvgagdvgksaltiqliq
SEQ ID NO: 10 G13R:
mteyklvvvgagrvgksaltiqliq
SEQ ID NO: 11 G13S:
mteyklvvvgagsvgksaltiqliq
SEQ ID NO: 12 G13V:
mteyklvvvgagvvgksaltiqliq
SEQ ID NO: 13 Q61E:
etclldildtageeeysamrdqymr
SEQ ID NO: 14 Q61H:
etclldildtagheeysamrdqymr
SEQ ID NO: 15 Q61K:
etclldildtagkeeysamrdqymr
SEQ ID NO: 16 Q61L:
etclldildtagleeysamrdqymr
SEQ ID NO: 17 Q61P:
etclldildtagpeeysamrdqymr
SEQ ID NO: 18 Q61R:
etclldildtagreeysamrdqymr
SEQ ID NO: 19 A146T:
qdlarsygipfietstktrqrvedafytlv
SEQ ID NO: 20 A146V:
qdlarsygipfietsvktrqrvedafytlv
SEQ ID NO: 21 A146P:
qdlarsygipfietspktrqrvedafytlv
SEQ ID NO: 22 G60D:
getclldildtadqeeysamrdqym
SEQ ID NO: 23 G60V:
getclldildtavqeeysamrdqym
SEQ ID NO: 24 G60R:
getclldildtarqeeysamrdqym
SEQ ID NO: 25 K117N:
dsedvpmvlvgnncdlpsrtvdtkq
SEQ ID NO: 26 K117R:
dsedvpmvlvgnrcdlpsrtvdtkq
SEQ ID NO: 27 A59T:
dgetclldildttgqeeysamrdqy
SEQ ID NO: 28 A59G:
dgetclldildtggqeeysamrdqy
SEQ ID NO: 29 A59E:
dgetclldildtegqeeysamrdqy
SEQ ID NO: 30 A59S:
dgetclldildtsgqeeysamrdqy
SEQ ID NO: 31 L19F:
vvvgaggvgksaftigliqnhfvde
NRAS
SEQ ID NO: 32 Q61R:
etclldildtagreeysamrdqymr
SEQ ID NO: 33 Q61K:
etclldildtagkeeysamrdqymr
SEQ ID NO: 34 Q61L:
etclldildtagleeysamrdqymr
SEQ ID NO: 35 Q61H:
etclldildtagheeysamrdqymr
SEQ ID NO: 36 Q61P:
etclldildtagpeeysamrdqymr
SEQ ID NO: 37 Q61E:
etclldildtageeeysamrdqymr
SEQ ID NO: 38 G12D:
mteyklvvvgadgvgksaltiqliq
SEQ ID NO: 39 G12A:
mteyklvvvgaagvgksaltiqliq
SEQ ID NO: 40 G12C:
mteyklvvvgacgvgksaltiqliq
SEQ ID NO: 41 G12R:
mteyklvvvgargvgksaltiqliq
SEQ ID NO: 42 G12S:
mteyklvvvgasgvgksaltiqliq
SEQ ID NO: 43 G12V:
mteyklvvvgavgvgksaltiqliq
SEQ ID NO: 44 G13C:
mteyklvvvgagcvgksaltiqliq
SEQ ID NO: 45 G13A:
mteyklvvvgagavgksaltiqliq
SEQ ID NO: 46 G13D:
mteyklvvvgagdvgksaltiqliq
SEQ ID NO: 47 G13R:
mteyklvvvgagrvgksaltiqliq
SEQ ID NO: 48 G13S:
mteyklvvvgagsvgksaltiqliq
SEQ ID NO: 49 G13V:
mteyklvvvgagvvgksaltiqliq
SEQ ID NO: 50 G60E:
getclldildtaeqeeysamrdqym
SEQ ID NO: 51 G60R:
getclldildtarqeeysamrdqym
SEQ ID NO: 52 G60V:
getclldildtavqeeysamrdqym
HRAS
SEQ ID NO: 53 Q61R:
etclldildtagreeysamrdqymr
SEQ ID NO: 54 Q61H:
etclldildtagheeysamrdqymr
SEQ ID NO: 55 Q61K:
etclldildtagkeeysamrdqymr
SEQ ID NO: 56 Q61L:
etclldildtagleeysamrdqymr
SEQ ID NO: 57 Q61P:
etclldildtagpeeysamrdqymr
SEQ ID NO: 58 G13R:
mteyklvvvgagrvgksaltiqliq
SEQ ID NO: 59 G13C:
mteyklvvvgagcvgksaltiqliq
SEQ ID NO: 60 G13D:
mteyklvvvgagdvgksaltiqliq
SEQ ID NO: 61 G13S:
mteyklvvvgagsvgksaltiqliq
SEQ ID NO: 62 G13V:
mteyklvvvgagvvgksaltiqliq
SEQ ID NO: 63 G12V:
mteyklvvvgavgvgksaltiqliq
SEQ ID NO: 64 G12A:
mteyklvvvgaagvgksaltiqliq
SEQ ID NO: 65 G12C:
mteyklvvvgacgvgksaltiqliq
SEQ ID NO: 66 G12D:
mteyklvvvgadgvgksaltiqliq
SEQ ID NO: 67 G12R:
mteyklvvvgargvgksaltiqliq
SEQ ID NO: 68 G12S:
mteyklvvvgasgvgksaltiqliq
SEQ ID NO: 69 K117N:
dsddvpmvlvgnncdlaartvesrq
PAN-RAS

SEQ ID NO:70 G12D, G13D, L19F, A59T, G60D, Q61H, K117N, A146T:
mteyklvvvg addvgksaft iqliqnhfvd eydptiedsy rkqvvidget clldildttd
heeysamrdq ymrtgegflc vfainntksf edihhyreqi krvkdsedvp mvlvgnncdl
psrtvdtkqa qdlarsygip fietstktrq rvedafytly reirqyfikk iskeektpgc
vkikkciim

In some embodiments, each of the ras derived peptides can be encoded by a single ORF. In other embodiments, the ras derived peptides can be encoded by more than one ORFs, such as two ORFs, three ORFs, or four ORFs, or more ORFs.

In some embodiments, the ORF encodes the polypeptide as set forth in SEQ ID NO: 70, or an equivalent thereof. In some embodiments, the equivalent of SEQ ID NO: 70 retains the following mutations: D aligned to the 12^{th} amino acid residue of SEQ ID NO: 70 (G12D), D aligned to the 13^{th} amino acid residue of SEQ ID NO: 70 (G13D); F aligned to the 19^{th} amino acid residue of SEQ ID NO: 70 (L19F); T aligned to the 59^{th} amino acid residue of SEQ ID NO: 70 (A59T); D aligned to the 60^{th} amino acid residue of SEQ ID NO: 70 (G60D); H aligned to the 61^{th} amino acid residue of SEQ ID NO: 70 (Q61H); N aligned to the 117^{th} amino acid residue of SEQ ID NO: 70 (K117N); and T aligned to the 146^{th} amino acid residue of SEQ ID NO: 70 (A146T).

In some embodiments, the ORF comprises, or consists essentially of, or yet further consists of the polynucleotide as set forth in AUGUUUGUUUUUCUUGUUUUAUUGCCACUAGUCUCUAGUCAGUGUAUG ACUGAAUAUAAACUUGUGGUAGUUGGAGCUGAUGACGUAGGCAAGAGU GCCUUUACGAUACAGCUAAUUCAGAAUCAUUUUGUGGACGAAUAUGAU CCAACAAUAGAGGAUUCCUACAGGAAGCAAGUAGUAAUUGAUGGAGAA ACCUGUCUCUUGGAUAUUCUCGACACAACAGAUCACGAGGAGUACAGU GCAAUGAGGGACCAGUACAUGAGGACUGGGGAGGGCUUUCUUUGUGUA UUUGCCAUAAAUAAUACUAAAUCAUUUGAAGAUAUUCACCAUUAUAGA GAACAAAUUAAAAGAGUUAAGGACUCUGAAGAUGUACCUAUGGUCCUA GUAGGAAAUAAUUGUGAUUUGCCUUCUAGAACAGUAGACACAAAACAG GCUCAGGACUUAGCAAGAAGUUAUGGAAUUCCUUUUAUUGAAACAUCA ACAAAGACAAGACAGAGAGUGGAGGAUGCUUUUUAUACAUUGGUGAGA GAGAUCCGACAAUACAGAUUGAAAAAAAUCAGCAAAGAAGAAAAGACU CCUGGCUGUGUGAAAAUUAAAAAAUGCAUUAUAAUGUAA (SEQ ID NO: 88), or nucleotide (nt) 1 to nt 612 of SEQ ID NO: 88, or an equivalent of each thereof encoding the same ras derived peptide.

In some embodiments, the ORF encodes a polypeptide comprising, or consisting essentially of, or yet further consisting of two or more (such as two, or three, or four, or five, or six, or seven, or eight, or nine, or ten, or more) ras derived peptides and an optionally peptide linker between any two adjacent ras derived peptides. In some embodiments, the linker comprises, or consists essentially of, or further consists of a peptide comprising about 1 aa to about 200 aa (including any integer or subrange within this range) of random amino acids. In some embodiments, the linker comprises, or consists essentially of, or yet further consists of a peptide as set forth in any one of SEQ ID NOs: 83-85. Additionally or alternatively, the linker comprises, or consists essentially of, or yet further consists of a cleavable peptide, such as a self-cleaving peptide.

In some embodiments, the encoded ras derived peptide or peptides comprise a wildtype residue (i.e., an unmutated residue, such as a glycine (G)) aligned to the 12^{th} amino acid residue of SEQ ID NO: 70, or a wildtype residue (i.e., an unmutated residue, such as G) aligned to the 13^{th} amino acid residue of SEQ ID NO: 70, or both.

In some embodiments, the ORF further encodes a signal peptide. In further embodiments, the signal peptide is located at the N terminus of the ras derived peptide, such as conjugated directly or indirectly to the N terminus of the ras derived peptide. In some embodiments, the single peptide is of or derived from a surface glycoprotein of a severe acute respiratory syndrome coronavirus 2 (SARS-CoV-2), or albumin, or an interleukin-2 (IL-2). In some embodiments, the signal peptide comprises, or consists essentially of, or yet further consists of MFVFLVLLPLVSSQC (SEQ ID NO: 87). In some embodiments, the signal peptide comprises, or consists essentially of, or yet further consists of MYRMQLLSCIALSLALVTNS (SEQ ID NO: 86).

In some embodiments, the RNA further comprises a 3′-UTR and a 5′-UTR. In some embodiments, the RNA further comprises one or more additional elements that stabilize the RNA and enhance expression of the peptides encoded by the ORF.

In some embodiments, the 5′-UTR comprises, or consists essentially of, or yet further comprises an m7G cap structure and a start codon. In some embodiments, the 5′-UTR comprises, or consists essentially of, or yet further comprises AGGACAUUUGCUUCUGACACAACUGUGUUCACUAGCAACCUCAAACAG ACACCGCCACC (SEQ ID NO: 89) or an equivalent thereof.

In some embodiments, the 3′-UTR comprises, or consists essentially of, or yet further comprises a stop codon and a polyA tail. In some embodiments, the 3′-UTR comprises, or consists essentially of, or yet further consists of GCUCGCUUUCUUGCUGUCCAAUUUCUAUUAAAGGUUCCUUUGUUCCCU AAGUCCAACUACUAAACUGGGGGAUAUUAUGAAGGGCCUUGAGCAUCU GGAUUCUGCCUAAUAAAAAACAUUUAUUUUCAUUGCCAAUAGGCCGAA AUCGGCAAGCGCGAUCGC (SEQ ID NO: 90) or an equivalent thereof.

In some embodiments, the RNA is prepared by transcribing a polynucleotide encoding the RNA in an in vitro transcription (IVT) system. In some embodiments, the RNA is prepared by transcribing a plasmid DNA (pDNA) vector encoding the RNA. In some embodiments, the vector is pUC57, or pSFV1, or pcDNA3, or pTK126. In some embodiments, the vector comprises, or consists essentially of, or yet further consists of TCGCGCGTTTCGGTGATGACGGTGAAAACCTCTGACACATGCAGCTCCCG GAGACGGTCACAGCTTGTCTGTAAGCGGATGCCGGGAGCAGACAAGCCC GTCAGGGCGCGTCAGCGGGTGTTGGCGGGTGTCGGGGCTGGCTTAACTAT GCGGCATCAGAGCAGATTGTACTGAGAGTGCACCATATGCGGTGTGAAAT ACCGCACAGATGCGTAAGGAGAAAATACCGCATCAGGCGCCATTCGCCAT TCAGGCTGCGCAACTGTTGGGAAGGGCGATCGGTGCGGGCCTCTTCGCTA TTACGCCAGCTGGCGAAAGGGGGATGTGCTGCAAGGCGATTAAGTTGGGT AACGCCAGGGTTTTCCCAGTCACGACGTTGTAAAACGACGGCCAGTGAAT TCGAGCTCGGTACCTCGCGAATGCATCTAGATATCGGATCCCGGGCCCGT CGACTGCAGAGGCCTGCATGCAAGCTTTAATACGACTCACTATAAGGACA TTTGCTTCTGACACAACTGTGTTCACTAGCAACCTCAAACAGACACCGCCA CCATGTTTGTTTTTCTTGTTTTATTGCCACTAGTCTCTAGTCAGTGTATGAC TGAATATAAACTTGTGGTAGTTGGAGCTGATGACGTAGGCAAGAGTGCCT TTACGATACAGCTAATTCAGAATCATTTTGTGGACGAATATGATCCAACA ATAGAGGATTCCTACAGGAAGCAAGTAGTAATTGATGGAGAAACCTGTCT CTTGGATATTCTCGACACAACAGATCACGAGGAGTACAGTGCAATGAGGG ACCAGTACATGAGGACTGGGGAGGGCTTTCTTTGTGTATTTGCCATAAATA ATACTAAATCATTTGAAGATATTCACCATTATAGAGAACAAATTAAAAGA GTTAAGGACTCTGAAGATGTACCTATGGTCCTAGTAGGAAATAATTGTGA TTTGCCTTCTAGAACAGTAGACACAAAACAGGCTCAGGACTTAGCAAGAA GTTATGGAATTCCTTTTATTGAAACATCAACAAAGACAAGACAGAGAGTG GAGGATGCTTTTTATACATTGGTGAGAGAGATCCGACAATACAGATTGAA AAAAATCAGCAAAGAAGAAAAGACTCCTGGCTGTGTGAAAATTAAAAAA TGCATTATAATGTAAGCTCGCTTTCTTGCTGTCCAATTTCTATTAAAGGTTC CTTTGTTCCCTAAGTCCAACTACTAAACTGGGGGATATTATGAAGGGCCTT GAGCATCTGGATTCTGCCTAATAAAAAACATTTATTTTCATTGCCAATAGG CCGAAATCGGCAAGCGCGATCGCAAAAAAAAAAAAAAAAAAAAAAAAA AAAAAAAAAAAAAAAAAAAAAAAAAAAAAAAAAAAGAAAAAAAAAAA AAAAAAAAAAAAAAAAAAAAAAAAAAAAAAAAAAAAAAAAAAAAAAA AAGAATTCCTCGAGGCGCGCCCGCTTCCTCGCTCACTGACTCGCTGCGCTC GGTCGTTCGGCTGCGGCGAGCGGTATCAGCTCACTCAAAGGCGGTAATAC GGTTATCCACAGAATCAGGGGATAACGCAGGAAAGAACATGTGAGCAAA AGGCCAGCAAAAGGCCAGGAACCGTAAAAAGGCCGCGTTGCTGGCGTTTT TCCATAGGCTCCGCCCCCCTGACGAGCATCACAAAAATCGACGCTCAAGT CAGAGGTGGCGAAACCCGACAGGACTATAAAGATACCAGGCGTTTCCCCC TGGAAGCTCCCTCGTGCGCTCTCCTGTTCCGACCCTGCCGCTTACCGGATA CCTGTCCGCCTTTCTCCCTTCGGGAAGCGTGGCGCTTTCTCATAGCTCACG CTGTAGGTATCTCAGTTCGGTGTAGGTCGTTCGCTCCAAGCTGGGCTGTGT GCACGAACCCCCCGTTCAGCCCGACCGCTGCGCCTTATCCGGTAACTATC GTCTTGAGTCCAACCCGGTAAGACACGACTTATCGCCACTGGCAGCAGCC ACTGGTAACAGGATTAGCAGAGCGAGGTATGTAGGCGGTGCTACAGAGTT CTTGAAGTGGTGGCCTAACTACGGCTACACTAGAAGAACAGTATTTGGTA TCTGCGCTCTGCTGAAGCCAGTTACCTTCGGAAAAAGAGTTGGTAGCTCTT GATCCGGCAAACAAACCACCGCTGGTAGCGGTGGTTTTTTTGTTTGCAAG CAGCAGATTACGCGCAGAAAAAAAGGATCTCAAGAAGATCCTTTGATCTT TTCTACGGGGTCTGACGCTCAGTGGAACGAAAACTCACGTTAAGGGATTT TGGTCATGAGATTATCAAAAAGGATCTTCACCTAGATCCTTTTAAATTAAA AATGAAGTTTTAAATCAAGCCCAATCTGAATAATGTTACAACCAATTAAC CAATTCTGATTAGAAAAACTCATCGAGCATCAAATGAAACTGCAATTTAT TCATATCAGGATTATCAATACCATATTTTTGAAAAAGCCGTTTCTGTAATG AAGGAGAAAACTCACCGAGGCAGTTCCATAGGATGGCAAGATCCTGGTAT CGGTCTGCGATTCCGACTCGTCCAACATCAATACAACCTATTAATTTCCCC TCGTCAAAAATAAGGTTATCAAGTGAGAAATCACCATGAGTGACGACTGA ATCCGGTGAGAATGGCAAAAGTTTATGCATTTCTTTCCAGACTTGTTCAAC AGGCCAGCCATTACGCTCGTCATCAAAATCACTCGCATCAACCAAACCGT TATTCATTCGTGATTGCGCCTGAGCGAGACGAAATACGCGATCGCTGTTA AAAGGACAATTACAAACAGGAATCGAATGCAACCGGCGCAGGAACACTG CCAGCGCATCAACAATATTTTCACCTGAATCAGGATATTCTTCTAATACCT GGAATGCTGTTTTTCCGGGGATCGCAGTGGTGAGTAACCATGCATCATCA GGAGTACGGATAAAATGCTTGATGGTCGGAAGAGGCATAAATTCCGTCAG CCAGTTTAGTCTGACCATCTCATCTGTAACATCATTGGCAACGCTACCTTT GCCATGTTTCAGAAACAACTCTGGCGCATCGGGCTTCCCATACAAGCGAT AGATTGTCGCACCTGATTGCCCGACATTATCGCGAGCCCATTTATACCCAT ATAAATCAGCATCCATGTTGGAATTTAATCGCGGCCTCGACGTTTCCCGTT GAATATGGCTCATAACACCCCTTGTATTACTGTTTATGTAAGCAGACAGTT TTATTGTTCATGATGATATATTTTTATCTTGTGCAATGTAACATCAGAGATT TTGAGACACGGGCCAGAGCTGCA (SEQ ID NO: 91) or an equivalent thereof. In some embodiments, the equivalent of SEQ ID NO: 91 still expresses the ras derived peptide.

In some embodiments, the RNA is a messenger RNA (mRNA).

In some embodiments, the GC content of the full-length RNA is about 35% to about 70% (including any percentage or any subranges within the range) of the total RNA content, such as about 40%, about 45%, about 50%, about 55%, about 60%, about 65%, or about 70%.

In some embodiments, the RNA is chemically modified. In some embodiments, the chemical modification comprises, or consists essentially or, or yet further consists of one or both of the incorporation of an N1-methyl-pseudouridine residue or a pseudouridine residue. In some embodiments, at least about 50% to about 100% of the uridine residues in the RNA are N1-methyl pseudouridine or pseudouridine. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at east about 90%, at least about 91%, at least about 92%, at least about 93%, at least about 94%, at least about 95%, at least about 96%, at least about 97%, at least about 98%, at least about 99%, or higher percentage of residues of the RNA is chemically modified by one or more of modifications as disclosed herein. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of uridine residues of the RNA is chemically modified by one or more of modifications as disclosed herein. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or higher percentage of uridine residues of the RNA is N1-methyl pseudouridine or pseudouridine.

In some embodiments, all or some of uridine residues are replaced by pseudouridines during in vitro transcription. This modification stabilizes the mRNA against enzymatic degradation in the cell, leading to enhanced translation efficiency of the mRNA. The pseudouridines used can be N1-methyl-pseudouridine, or other modifications that are well known in the art such as N6m -ethyladenosine (m6A), inosine, pseudouridine, 5-methylcytidine (m5C), 5-hydroxymethylcytidine (hm5C), and N1-methyladenosine (m1A). The modification optionally is made throughout the entire mRNA. The skilled artisan will recognize that other modified RNA residues can be used to stabilize the protein's 3 dimensional structure and increase protein translation.

Further provided is a polynucleotide encoding an RNA as disclosed herein, or a polynucleotide complementary thereto, or both. In some embodiments, the polynucleotide is selected from the group of: a deoxyribonucleic acid (DNA), an RNA, a hybrid of DNA and RNA, or an analog of each thereof. In further embodiments, the analog comprises, or consists essentially of, or yet further consists of a peptide nucleic acid or a locked nucleic acid or both.

In some embodiments, the polynucleotide further comprises a regulatory sequence directing the transcription thereof. In some embodiments, the regulatory sequence is suitable for use in an in vitro transcription system. In further embodiments, the regulatory sequence comprises, or consists essentially of, or yet further consists of a promotor. In yet further embodiments, the promoter comprises, or consists essentially of, or yet further consists of: a bacteriophage RNA polymerase promoter, such as a T7 promoter, or a SP6 promoter, or a T3 promoter. In some embodiments, the polynucleotide comprises a marker selected from a detectable marker, a purification marker, or a selection marker.

In a further aspect, provided is a vector comprising, or consisting essentially of, or yet further consisting of a polynucleotide as disclosed herein.

In some embodiments, the vector further comprises a regulatory sequence operatively linked to the polynucleotide to direct the transcription thereof. In some embodiments, the regulatory sequence is suitable for use in an in vitro transcription system. In further embodiments, the regulatory sequence comprises, or consists essentially of, or yet further consists of a promotor. In yet further embodiments, the promoter comprises, or consists essentially of, or yet further consists of: a bacteriophage RNA polymerase promoter, such as a T7 promoter, or a SP6 promoter, or a T3 promoter. In some embodiments, the vector further comprises a marker selected from a detectable marker, a purification marker, or a selection marker.

In some embodiments, the vector further comprises a regulatory sequence operatively linked to the polynucleotide to direct the replication thereof. In further embodiments, the regulatory sequence comprises, or alternatively consists essentially of, or yet further consists of one or more of the following: an origin of replication or a primer annealing site, a promoter, or an enhancer.

In some embodiments, the vector is a non-viral vector. In further embodiments, the non-viral vector is a plasmid, or a liposome, or a micelle. In some embodiments, the vector is pUC57, or pSFV1, or pcDNA3, or pTK126, or another plasmid available at addgene or Standard European Vector Architecture (SEVA). In some embodiments, the vector comprises, or consists essentially of, or yet further consists of SEQ ID NO: 91 or an equivalent thereof. In some embodiments, the equivalent of SEQ ID NO: 91 still expresses the ras derived peptide.

In some embodiments, the vector is a viral vector. In further embodiments, the viral vector is selected from the group consisting of an adenoviral vector, or an adeno-associated viral vector, or a retroviral vector, or a lentiviral vector, or a plant viral vector.

In yet a further aspect, provided is a cell comprising one or more of: an RNA as disclosed herein, a polynucleotide as disclosed herein, or a vector as disclosed herein. In some embodiments, the cell is suitable for replicating any one or more of: the RNA, the polynucleotide, or the vector, thereby producing the one or more of: the RNA, the polynucleotide, or the vector. In some embodiments, the cell is suitable for transcribing the polynucleotide or the vector to the RNA, thereby producing the RNA.

In some embodiments, the cell is a prokaryotic cell. In further embodiments, the prokaryotic cell is an Escherichia coli cell.

In some embodiments, the cell is a eukaryotic cell. In further embodiments, the eukaryotic cell is any one of a mammal cell, an insect cell, or a yeast cell.

In some embodiments, a cell as disclosed herein is suitable for producing (such as transcribing or expressing) an RNA as disclosed herein. Such production can be in vivo or in vitro. For example, the cell can be used to produce the RNA in vitro. Such RNA is then administrated to a subject in need thereof optionally with a suitable pharmaceutical acceptable carrier. Alternatively, the cell can be used as a cell therapy and directly administrated to a subject in need thereof optionally with a suitable pharmaceutical acceptable carrier. In further embodiments, the cell therapy can additionally deliver other prophylactic or therapeutic agent to the subject. In some embodiments, the cell used as a cell therapy is an immune cell, such as a T cell, a B cell, an NK cell, an NKT cell, a dendritic cell, a myeloid cell, a monocyte, or a macrophage.

In one aspect, provided is a composition comprising, or consisting essentially of, or yet further consisting of a carrier, and one or more of: an RNA as disclosed herein, a polynucleotide as disclosed herein, a vector as disclosed herein, or a cell as disclosed herein. In some embodiments, the carrier is a pharmaceutically acceptable carrier. In some embodiments, the composition further comprises an additional anti-cancer therapy. Additionally or alternatively, the composition further comprises an adjuvant.

In a further aspect, provided is a method of producing an RNA, such as those as disclosed herein. In some embodiments, the method comprises, or consists essentially of, or yet further consists of culturing a cell as disclosed herein under conditions suitable for expressing the RNA (such as transcribing a DNA to the RNA). In some embodiments, the cell comprises the DNA encoding the RNA of the disclosure. In some embodiments, the method comprises, or consists essentially of, or yet further consists of contacting a polynucleotide as disclosed herein or a vector as disclosed herein with an RNA polymerase, adenosine triphosphate (ATP), cytidine triphosphate (CTP), guanosine-5′-triphosphate (GTP), and uridine triphosphate (UTP) or a chemically modified UTP under conditions suitable for expressing the RNA (such as transcribing a DNA to the RNA). In some embodiments, the method further comprises isolating the RNA. In some embodiments, the method further comprises storing the RNA.

In yet a further aspect, provided is an RNA produced by a method as disclosed herein, or a composition comprising, or consisting essentially of, or yet further consisting of the produced RNA.

Improving mRNA Vaccine Expression Efficiency

To improve the mRNA vaccine expression efficiency in the mammalian cells, mRNA stability can be enhanced by partial chemical modification. To further increase the translation efficiency, short and double strand RNAs derived from aberrant RNA polymerase activities are removed. To improve the potency of mRNA vaccines, sequence optimization can be used, together with usage of modified nucleosides, such as pseudouridine (φ), 5-methylcytidine (5mC), Cap-1 structure and optimized codons, which in turn improve translation efficiency. During in vitro transcription of mRNA, immature mRNA can be produced as a contaminant which inhibits translation through stimulating innate immune activation. FPLC and HPLC purification can be used to remove these contaminants.

In a composition presented herein, the template for in vitro transcription of mRNA contains five cis-acting structural elements, namely from 5′ to 3′ end: (i) an optimized cap structure, (ii) an optimized 5′ untranslated region (UTR), (iii) a codon optimized coding sequence, (iv) an optimized 3′ UTR and (v) a stretch of repeated adenine nucleotides (polyA tail) (FIG. 5). These cis-acting structural elements are further optimized in the endeavor for better mRNA features. Provided herein, the 5′-UTR includes a start codon and some other elements, but does not encode polypeptide (i.e. it is non-coding). In some embodiments, a 5′-UTR of the present disclosure comprises, or consists essentially of, or yet further consists of a cap structure with 7-methylguanosine (7mG) sequences. The 3′-UTR is directly downstream (3′) from the stop codon (the codon of an mRNA transcript representing a termination signal) and does not encode a polypeptide (is non-coding). A polyA tail is a special region of mRNA that is downstream from 3′-UTR and contains multiple consecutive adenosine monophosphates.

A typical mRNA production cassette comprises, or consists essentially of, or yet further consists of a Cap structure at its 5′-UTR region, followed by an in-frame mRNA sequence coding for a corresponding protein or peptide. In some embodiments, 3′-UTR with polyA tail is required for efficient mRNA production. In some embodiments, an expression cassette is used not only for efficiency of mRNA production but also for the subsequent protein or peptide production (FIG. 5).

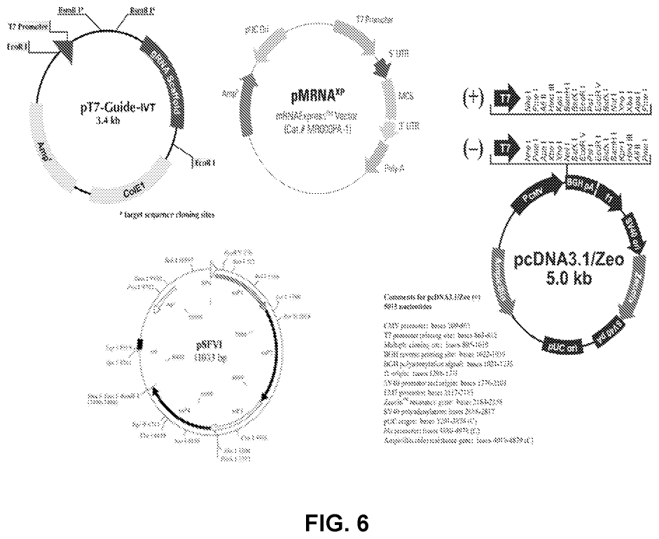

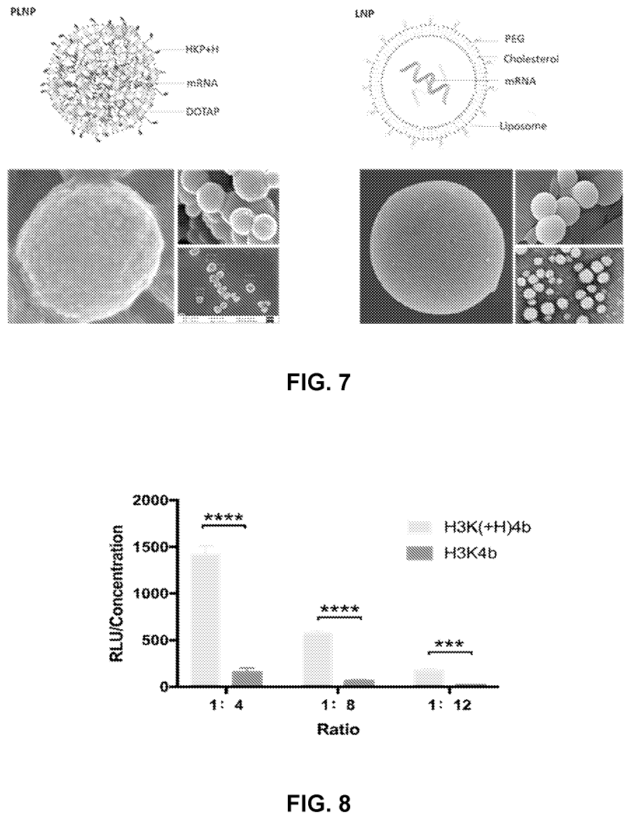

In some embodiments, mRNA is produced by in vitro transcription (IVT) from a linear DNA template containing a bacteriophage promoter, the optimized UTR's and the codon optimized sequence by using an RNA polymerase (T7, T3 or SP6) and a mix of the different nucleosides. In other embodiments, the linear DNA template can be cloned into a plasmid DNA (pDNA) as a delivery vector. The plasmid vectors can be adapted for mRNA vaccine production. Commonly used plasmids include pSFV1, pcDNA3 and pTK126 (FIG. 6). One unique mRNA expression system is pEVL (see Grier et al. Mol Ther Nucleic Acids. 19; 5:e306 (2016), “pEVL: A Linear Plasmid for Generating mRNA IVT Templates With Extended Encoded Poly(A) Sequences,” the disclosure of which is incorporated herein by reference in its entirety).

In some embodiments, the vaccine comprises, or consists essentially of, or yet further consists of an effective amount of an mRNA, which comprises, or consists essentially of, or yet further consists of an open reading frame encoding one or more of ras neoantigens, or other neoantigens and a pharmaceutically acceptable carrier. The effective amount is an amount effective to induce in the subject a neoantigen-specific, such as ras-specific, immune response. In one embodiment, the carrier comprises, or consists essentially of, or yet further consists of a polymeric nanoparticle or a liposomal nanoparticle. In some embodiments, the carrier is a Histidine-Lysine-copolymer or a Spermine-Liposome Conjugate. In some embodiments, the carrier further comprises DOTAP or MC3 or both.

In some embodiments, the vaccine comprises, or consists essentially of, or yet further consists of an effective amount of an mRNA, which comprises, or consists essentially of, or yet further consists of an open reading frame encoding multiple neoantigens separated by self-cleaving 2A peptide sites, signal sequences to incorporate the neoantigen into the membrane and/or be secreted using different signal sequences, such as the albumin signal sequence.

Histidine-Lysine (HK) Polypeptides as mRNA Vaccine Delivery Systems

Despite significant progress in the rational design of mRNA vaccines and elucidation of their mechanism of action during the past few years, their widespread application is limited by the presence of ubiquitous ribonucleases (RNases), as well as the need to facilitate vaccine entry into cells and subsequent escape from endosomes, and to target them to lymphoid organs or particular cells. See, for example, Midoux and Pichon, Expert Rev Vaccines. 2015; 14(2): 221-34. mRNA formulations with chemical carriers provide more specificity and internalization in dendritic cells (DCs) for better immune responses and dose reduction.

A non-viral delivery system is more advantageous than the viral delivery system. See, for example, Brito et al. Adv Genet. 2015; 89: 179-233. One non-limiting examples is that non-viral methods are preferred over viral delivery systems for their safety and cost-effectiveness. See, for example, Juliano et al. Nucleic Acids Res. 2008; 36: 4158-4171. Non-viral methods for delivery of vaccines include naked mRNA vaccines, gene gun, protamine condensation, adjuvant based vaccines, and encapsulated mRNA vaccines. Positive-sense RNA viruses, alpha viruses can be used for the viral delivery system. The glycoproteins (E1 and E2) of alpha virus can be used for endosomal escape and cell targeting in the host. In addition to direct delivery by viral or non-viral mediated methods, ex vivo transfected mRNA is an alternative to naked mRNA vaccination. In this method, mRNAs are transfected into monocytes, macrophages, T cells, dendritic cells (DCs) and mesenchymal stem cells (MSC), see, for example, Sahin et al., Nat Rev Drug Discov. 2014; 13: 759-780, before administration. A strong immune response can be induced by ex vivo transfected mRNA vaccination when compared to naked mRNA vaccination, which offers only optimal expression.

As described herein, a series of branched Histidine-Lysine (HK) polypeptides (HKP) can be applied to encapsulate mRNAs by electrostatic action. The HKPs used herein are a group of linear and branched peptides that consist of histidine and lysine residues and these peptides, in most cases, form spherical nanoparticles when mixed with nucleic acids. Such polypeptides are disclosed in U.S. Pat. No. 7,070,807 B2, issued Jul. 4, 2006, and in U.S. Pat. No. 7,163,695 B2, issued Jan. 16, 2007. The disclosures of each of these patents are incorporated herein by reference in their entireties. Similar to other carriers, HKP carriers differ in their ability to carry various nucleic acids. For instance, the four-branched HK peptide (H2K4b) is a good carrier of plasmids (see, for example, Chen, et al., Nucleic Acids Res. 2001; 29: 1334-1340; and Zhang et al., Methods Mol Biol. 2004; 245: 33-52), but is a poor carrier for siRNA. In addition, H3K4b, H3K(+H)4b, and H3K8b are excellent carriers of siRNA (see, for example, Leng et al., J Gene Med. 2005; 7: 977-986), but only H3K(+H)4b shows effectiveness in carrying mRNA into the targeted cells. (See FIG. 8) Furthermore, H3K(+H)4b is a more effective carrier of mRNA than DOTAP liposomes. In addition, a delivery carrier combination of H3K(+H)4b, MC3 and/or DOTAP can be used as described herein to enhance the efficacy of mRNA delivery. The results as described herein showed that the H3k(+H)4b, MC3 and/or DOTAP combination was the most effective carrier of mRNA. This combination was synergistic for its ability to carry mRNA into cells (FIGS. 8-12).

### Formulation and Related Methods

Accordingly, in one aspect, provided is a composition (such as an immunogenic composition) comprising, or consisting essentially of, or yet further consisting of, for example an effective amount of, an RNA as disclosed herein formulated in a pharmaceutically acceptable carrier. In some embodiments, the composition comprises, or consists essentially of, or yet further consists of the RNA and the pharmaceutically acceptable carrier.

In some embodiments, the pharmaceutically acceptable carrier comprises, or consists essentially of, or yet further consists of a nanoparticle. In some embodiments, the nanoparticle is a polymeric nanoparticle or a liposomal nanoparticle or both. In some embodiments, the nanoparticle is a lipid nanoparticle (LNP). In some embodiments, the pharmaceutically acceptable carrier comprises, or consists essentially of, or yet further consists of a polymeric nanoparticle or a liposomal nanoparticle or both.

In some embodiments, the polymeric nanoparticle carrier comprises, or consists essentially of, or yet further consists of a Histidine-Lysine co-polymer (HKP). In further embodiments, the HKP comprises, or consists essentially of, or yet further consists of H3K(+H)4b. In yet further embodiments, the HKP comprises, or consists essentially of, or yet further consists of H3k(+H)4b. In some embodiments, the HKP comprises a side chain selected from SEQ ID NOs: 72-81.

In some embodiments, the mass ratio of HKP and the RNA in the composition is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of HKP and the RNA in the composition is about 2.5:1. In another embodiment, the mass ratio of HKP and the RNA in the composition is about 4:1.

In some embodiments, the polymeric nanoparticle carrier further comprises a lipid. In further embodiments, the lipid is a cationic lipid. In yet further embodiments, the cationic lipid is ionizable.

In some embodiments, the cationic lipid comprises, or consists essentially of, or yet further consists of Dlin-MC3-DMA (MC3) or dioleoyloxy-3-(trimethylammonio)propane (DOTAP) or both.

In some embodiments, the lipid further comprises one or more of: a helper lipid, a cholesterol, or a PEGylated lipid. In some embodiments, the lipid further comprises PLA or PLGA.

In some embodiments, the HKP and the mRNA self-assemble into nanoparticles upon admixture.

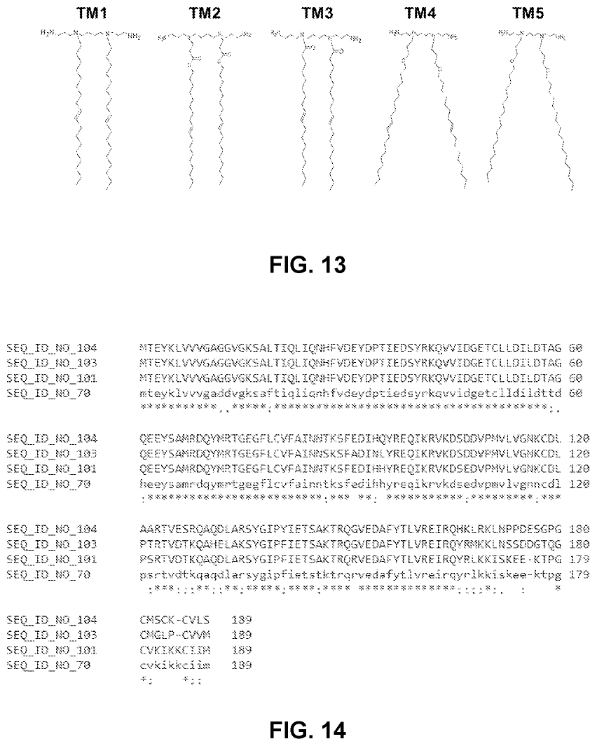

In some embodiments, the liposomal nanoparticle carrier comprises, or consists essentially of, or yet further consists of a Spermine-Lipid Cholesterol (SLiC). In further embodiments, the SLiC is selected from the group consisting of TM1-TM5, the structures of which are illustrated in FIG. 13.

In some embodiments, the pharmaceutical acceptable carrier is a lipid nanoparticle (LNP). In some embodiments, the lipid is a cationic lipid. In further embodiments, the cationic lipid is ionizable. In some embodiments, the LNP comprises, or consists essentially of, or yet further consists of one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof. In some embodiments, the LNP further comprises one or more of: a helper lipid, a cholesterol, or a PEGylated lipid.

In some embodiments, the mass ratio of LNP and the RNA in the composition is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of LNP and the RNA in the composition is about 2.5:1. In another embodiment, the mass ratio of LNP and the RNA in the composition is about 4:1.

In some embodiments, the helper lipid comprises, or consists essentially of, or yet further consists of one or more of: disteroylphosphatidyl choline (DSPC), Dipalmitoylphosphatidylcholine (DPPC), (2R)-3-(Hexadecanoyloxy)-2-{[(9Z)-octadec-9-enoyl]oxy}propyl 2-(trimethylazaniumyl)ethyl phosphate (POPC), or dioleoyl phosphatidylethanolamine (DOPE).

In some embodiments, the cholesterol comprises, or consists essentially of, or yet further consists of a plant cholesterol or an animal cholesterol or both.

In some embodiments, the PEGylated lipid comprises, or consists essentially of, or yet further consists of one or more of: PEG-c-DOMG (R-3-[(ω-methoxy-poly(ethyleneglycol)2000)carbamoyl)]-1,2-dimyristyloxypropyl-3-amine), PEG-DSG (1,2-Distearoyl-sn-glycerol, methoxypolyethylene glycol), PEG-DMG (1,2-Dimyristoyl-sn-glycerol) optionally PEG2000-DMG ((1,2-dimyristoyl-sn-glycero-3-phosphoethanolamine-N-[methoxy(polyethylene glycol)-2000)], or PEG-DPG (1,2-Dipalmitoyl-sn-glycerol, methoxypolyethylene glycol).

In some embodiments, the mass ratio of the cationic lipid and the helper lipid is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and the helper lipid is about 1:1.

In some embodiments, the mass ratio of the cationic lipid and cholesterol is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and cholesterol is about 1:1.

In some embodiments, the mass ratio of the cationic lipid and PEGylated lipid is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the cationic lipid and PEGylated lipid is about 1:1.

The mass ratio of the cationic lipid, helper lipid, cholesterol and PEGylated lipid can be calculated by one of skill in the art based on the ratios of the cationic lipid and the helper lipid, the cationic lipid and the cholesterol and the cationic lipid and the PEGylated lipid as disclosed herein.

In some embodiments, the LNP comprises, or consists essentially of, or yet further consists of SM-102, DSPC, cholesterol and PEG2000-DMG. In some embodiments, the mass ratio of the SM-102, DSPC, cholesterol and PEG200-DMG is about 1:1:1:1. In some embodiments, the molar ratio of the SM-102, DSPC, cholesterol and PEG2000-DMG is about 50:10:38.5:1.5.

In some embodiments, a mass ratio as provided here can be substituted with another parameter, such as a molar ratio, a weight percentage over the total weight, a component's weight over the total volume, or a molar percentage over the total molar amount. Knowing the component and its molecular weight, one of skill in the art would have no difficulty in converting a mass ratio to a molar ratio or other equivalent parameters.

In a further aspect, provided is a method of producing a composition as disclosed herein. The method comprises, or consists essentially of, or yet further consists of contacting an RNA as disclosed herein with an HKP, thereby the RNA and the HKP are self-assembled into nanoparticles.

In some embodiments, the mass ratio of HKP and the RNA in the contacting step is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of HKP and the RNA in the contacting step is about 2.5:1. In another embodiment, the mass ratio of HKP and the RNA in the contacting step is about 4:1.

In some embodiments, the method further comprises contacting the HKP and RNA with a cationic lipid. In further embodiments, the cationic lipid comprises, or consists essentially of, or yet further consists of Dlin-MC3-DMA (MC3) or DOTAP (dioleoyloxy-3-(trimethylammonio)propane) or both. In yet further embodiments, the mass ratio of the cationic lipid and the RNA in the contacting step is about 10:1 to about 1:10, including any range or ratio there between, for example, about 5:1 to 1:5, about 5:1 to 1:1, about 10:1, about 9.5:1, about 9:1, about 8.5:1, about 8:1, about 7.5:1, about 7:1, about 6.5:1, about 6:1, about 5.5:1, about 5:1, about 4.5:1, about 4:1, about 3.5:1, about 3:1, about 2:5:1, about 2:1, about 1.5:1, about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the mass ratio of the RNA and the cationic lipid in the contacting step is about 1:1. Accordingly, the mass ratio of the HKP, the RNA and the cationic lipid in the contacting step can be calculated based on the ratio between the HKP and the RNA and the ratio between the RNA and the cationic lipid. For example, if the ratio of the HKP to the RNA is about 4:1 and the ratio of the RNA to the cationic lipid is about 1:1, the ratio of the HKP to the RNA to the cationic lipid is about 4:1:1.

In yet a further aspect, provided is a method of producing a composition as disclosed herein. The method comprises, or consists essentially of, or yet further consists of contacting an RNA as disclosed herein with a lipid, thereby the RNA and the lipid are self-assembled into lipid nanoparticles (LNPs).

In some embodiments, the LNPs comprise, or consist essentially of, or yet further consist of one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof.

In some embodiments, the LNPs further comprise one or more of: a helper lipid, a cholesterol, or a PEGylated lipid. In some embodiments, the helper lipid comprises, or consists essentially of, or yet further consists of one or more of: disteroylphosphatidyl choline (DSPC), Dipalmitoylphosphatidylcholine (DPPC), (2R)-3-(Hexadecanoyloxy)-2-{[(9Z)-octadec-9-enoyl]oxy}propyl 2-(trimethylazaniumyl)ethyl phosphate (POPC), or dioleoyl phosphatidylethanolamine (DOPE). In some embodiments, the cholesterol comprises, or consists essentially of, or yet further consists of a plant cholesterol or an animal cholesterol or both. In some embodiments, the PEGylated lipid comprises, or consists essentially of, or yet further consists of one or more of: PEG-c-DOMG (R-3-[(ω-methoxy-poly(ethyleneglycol)2000)carbamoyl)]-1,2-dimyristyloxypropyl-3-amine), PEG-DSG (1,2-Distearoyl-sn-glycerol, methoxypolyethylene glycol), PEG-DMG (1,2-Dimyristoyl-sn-glycerol) optionally PEG2000-DMG ((1,2-dimyristoyl-sn-glycero-3-phosphoethanolamine-N-[methoxy(polyethylene glycol)-2000)], or PEG-DPG (1,2-Dipalmitoyl-sn-glycerol, methoxypolyethylene glycol).

In some embodiments, the LNPs comprise, or consist essentially of, or yet further consist of SM-102, DSPC, cholesterol and PEG2000-DMG. In some embodiments, the mass ratio of the SM-102, DSPC, cholesterol and PEG200-DMG is about 1:1:1:1. Additionally or alternatively, the molar ratio of the SM-102, DSPC, cholesterol and PEG2000-DMG is about 50:10:38.5:1.5.

In some embodiments, the contacting step is performed in a microfluidic mixer. In further embodiments, the microfluidic mixer is a slit interdigital micromixer, or a staggered herringbone micromixer (SHM).

Also provided is a composition produced by a method as disclosed herein.

### Method of Treatment

Also provided is a method of treating a subject having a cancer, or at risk of having a cancer, or suspect of having a cancer. In some embodiments, the cancer comprises a ras mutation as disclosed herein. In some embodiments, the ras mutation is a mutation of the ras gene. In some embodiments, the ras mutation is a mutation of the RAS protein. In some embodiments, the cancer comprises a mutated ras gene encoding an amino acid RAS mutation as disclosed herein. In further embodiments, the cancer comprises any one or more of: a mutation of SEQ ID NOs: 1 to 69. Methods to determine when the method is successful are known in the art and briefly described herein.

Further provided is a method of inhibiting the growth of a tumor or cancer cell. The method comprises, or consists essentially of, or yet further consists of contacting an immune cell with any one or more of an RNA as disclosed herein, a polynucleotide as disclosed herein, a vector as disclosed herein, a cell as disclosed herein, or a composition as disclosed herein, thereby activating the immune cell, and contacting the tumor or cancer cell with the activated immune cell. In some embodiments, the cancer cell or tumor comprises a ras mutation as disclosed herein. In some embodiments, the ras mutation is a mutation of the ras gene. In some embodiments, the ras mutation is a mutation of the RAS protein. In some embodiments, the cancer comprises a mutated ras gene encoding an amino acid RAS mutation as disclosed herein. In further embodiments, the cancer comprises any one or more of: a mutation of SEQ ID NOs: 1 to 69. Either or both of the contacting steps can be in vitro or in vivo.

Additionally or alternatively, provided is a screening method or a screening step of a method as disclosed herein for personalized or precision method, or alternatively to test for new combination therapies. The method comprises, or consists essentially of, or yet further consists of detecting a mutation as disclosed herein. In some embodiments, a mutation of the ras gene can be detected using sequencing, southern blots, or northern blots. In some embodiments, a mutation of the ras protein can be detected using flow cytometry or western blots. The method can be practiced in an animal to produce an animal model for treatment or to treat an animal, as determined by a treating veterinarian. Methods to determine when the method is successful are known in the art and briefly described herein.

In some embodiments, the cancer is an adenocarcinoma, an adenocarcinoma, an adenoma, a leukemia, a lymphoma, a carcinoma, a melanoma, an angiosarcoma, a pancreatic cancer, a colon cancer, a colorectal cancer, a rectal cancer, or a seminoma. The cancer can be primary or metastatic. The subject in need thereof may be suffering from an active cancer or be in remission, or at risk of developing a cancer, primary or secondary.

Additionally or alternatively, provided is a method for inducing an immune response, for example to a ras mutation as disclosed herein, in a subject in need thereof. In some embodiments, the immune response comprises, or consists essentially of, or yet further consists of any one or more of: an Th1 immune response, activation of CD8+ T cells, or production of a pro-inflammatory cytokine, such as interleukin-2 (IL-2), interferon-gamma (IFN-γ), or tumor necrosis factor-beta (TNF-β). Methods to determine when the method is successful are known in the art and briefly described herein.

These methods comprise, or consist essentially of, or yet further consist of administering to the subject, for example an effective amount of (e.g., a pharmaceutically effective amount of), any one or more of: an RNA as disclosed herein, a polynucleotide as disclosed herein, a vector as disclosed herein, a cell as disclosed herein, or a composition as disclosed herein.

In some embodiments, the RNA encodes SEQ ID NO: 70. In further embodiments, the RNA further encodes a signal peptide as set forth in SEQ ID NO: 87 which is conjugated to the N terminus of SEQ ID NO: 70. In some embodiments, the RNA comprises, or consists essentially of, or yet further consists of SEQ ID NO: 88 or (nt) 1 to nt 612 of SEQ ID NO: 88. In further embodiments, the RNA further comprise a 5′UTR (for example comprising, or consisting essentially of, or yet further consisting of SEQ ID NO: 89) and a 3′UTR (for example comprising, or consisting essentially of, or yet further consisting of SEQ ID NO: 90). In some embodiments, the vector comprises, or consists essentially of, or yet further consists of SEQ ID NO: 91. In some embodiments, the composition comprises the RNA formulated in a carrier, such as an LNP or a HKP nanoparticle as disclosed herein.

In some embodiments, the administration is intratumoral, or intravenous, or intramuscular, or intradermal, or subcutaneous.

In some embodiments, the subject is a mammal, or a human.

In some embodiments, the method further comprises administering to the subject an additional anti-cancer therapy. In some embodiments, the anti-cancer therapy is administrated prior to, or concurrently with, or after the administration of any one or more of the following: the RNA as disclosed herein, the polynucleotide as disclosed herein, the vector as disclosed herein, the cell as disclosed herein, or the composition as disclosed herein.

In some embodiments, the administration was repeated for at least one time, at least two times, at least three times, at least four times, or more. In further embodiments, the interval between any two administrations can be 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer.

In some embodiments, the method further comprises detecting a ras mutation as disclosed herein in a biological sample of the subject, such as a tumor biopsy or a circulating tumor DNA, prior to the administration.

In some embodiments, the ras mutation is a mutation of the ras gene. In some embodiments, the ras mutation is a mutation of the RAS protein.

In some embodiments, the method further comprises monitoring a ras mutation as disclosed herein in a biological sample of the subject, such as a tumor biopsy or a circulating tumor DNA, after the administration.

In some embodiments, the ras mutation is a mutation of the ras gene. In some embodiments, the ras mutation is a mutation of the RAS protein.

In some embodiments, the method further comprises detecting antibodies recognizing and binding to a ras mutation as disclosed herein in a biological sample of the subject, such as a blood sample, after the administration.

As used herein, an effective dose of an RNA, or polynucleotide, or vector, or cell or composition as disclosed herein is the dose required to produce a protective immune response in the subject to be administered. A protective immune response in the present context is one that treats a cancer in a subject. The RNA, or polynucleotide, or vector, or cell or composition as disclosed herein can be administered one or more times. An initial measurement of an immune response to the vaccine may be made by measuring production of antibodies in the subject receiving the RNA, or polynucleotide, or vector, or cell, or composition. Methods of measuring antibody production in this manner are also well known in the art, is that dose required to prevent, inhibit the occurrence, or treat (alleviate a symptom to some extent, preferably all of the symptoms) of a cancer. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the formulated composition.

In some embodiments, the RNA compositions can be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic or prophylactic effect. In some embodiments, the RNA composition is administered at a dosage of about 10 to about 500 μg/kg of body weight, or any dosage or subranges therein, such as about 28.5-285 μg/kg of body weight. The desired dosage can be delivered three times a day, two times a day, once a day, every other day, every third day, every week, every two weeks, every three weeks, every four weeks, every 2 months, every three months, every 6 months, etc. In certain embodiments, the desired dosage can be delivered using multiple administrations (e.g., two, three, four, five, six, seven, eight, nine, ten, eleven, twelve, thirteen, fourteen, or more administrations). When multiple administrations are employed, split dosing regimens such as those described herein can be used. In some embodiments, the RNA compositions can be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg. In some embodiments, the RNA compositions can be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the RNA compositions can be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a the RNA composition can be administered three or four times.

### Kits

In one aspect, provided is a kit for use in a method as disclosed herein.

In some embodiments, the kit comprises, or alternatively consists essentially of, or yet further consist of instructions for use and one or more of: an RNA as disclosed herein, a polynucleotide as disclosed herein, a vector as disclosed herein, a cell as disclosed herein, or a composition as disclosed herein. In further embodiments, the kit is suitable for use in a method of treatment as disclosed herein. In some embodiments, the kit further comprises an anti-cancer therapy.

In some embodiments, the kit comprises, or alternatively consists essentially of, or yet further consist of instructions for use and one or more of: an RNA as disclosed herein, a polynucleotide as disclosed herein, a vector as disclosed herein, a cell as disclosed herein, a composition as disclosed herein, an HKP, or a lipid optionally a cationic lipid. In further embodiments, the kit is suitable for use in a method producing an RNA or a composition as disclosed herein.

In some embodiments, the kit comprises, or alternatively consists essentially of, or yet further consist of instructions of use, a polynucleotide or a vector as disclosed herein, an RNA polymerase, ATP, CTP, GTP, and UTP or a chemically modified UTP. In further embodiments, the kit is suitable for use in an in vitro method producing an RNA or a composition as disclosed herein.

### EXPERIMENTAL METHODS

The following examples are illustrative of procedures which can be used in various instances in carrying the disclosure into effect.

### Example 1: Design mRNA Targeting Kras Mutations

As described herein, the vaccine comprises, or consists essentially of, or yet further consists of a synthetic mRNA containing a whole or part of a protein-encoding open reading frame (ORF). Optimally, the ORF is flanked by two elements: a “cap,” i.e., a 7-methyl-guanosine residue joined to the 5′-end via a 5′-5′ triphosphate, and a polyA tail at the 3′-end. In some embodiments, the mRNA vaccines are linear RNA fragments including additional components. Such mRNA vaccines were constructed. A single chromatographic step was performed to ensure that mRNA was separated according to size and to remove both shorter and longer transcripts, yielding a pure single mRNA product.

In other embodiments, the mRNA vaccine is a vector-based expression system, which comprises, or consists essentially of, or yet further consists of a promoter, an ORF, optionally a poly(d(A/T)) sequence transcribed into polyA and a unique restriction site for linearization of the vector to ensure defined termination of transcription (the cap is not encoded by the template). Such vectors were constructed. In order to facilitate the operation, DNA fragments corresponding to individual ras neoantigens were cloned into a specific vector as tandem minigenes (FIG. 4). RNA molecules transcribed from this vector can be translated into a polypeptide through an in vitro expression system (FIG. 5) and function as an mRNA pan ras vaccine.

### Example 2: mRNA Transfection into Cells and Measurement of mRNA Expression In Vitro

The mRNA constructs expressing epitopes of ras neoantigens were transfected into human cells in vitro using a variety of commercially available transfection reagents. Cells used for these studies included Huh7, Vero cells, A549 cells and others. Electroporation (using technology from MaxCyte, Gaithersburg, MD) was also examined as an option for delivery. The various delivery processes were tested and compared in order to determine the one having good uptake into a variety of cells and to evaluate subsequent expression of the construct. The protein production by each construct was determined and it was also determined whether the product is secreted from the cells. mRNA was detected in live cells using SmartFlare probes (Millipore) or using Q-RT-PCR.

### Example 3: Detection of mRNA Uptaking into Cells Using SmartFlare Technology

SmartFlare probes have recently emerged as a promising tool for visualization and quantification of specific RNAs in living cells. These smart flares are beads that have a sequence attached that, when recognizing the RNA sequence in the cell, produce an increase in fluorescence. Smartflares (Merck) were designed against several regions along the constructs in case steric hindrance reduces signal from one region.

Vero or other cells were cultured in collagen-coated 24-well glass-bottom plates at a concentration of 1×10⁴ cells per well, in 1 ml of RPMI-1640 for 12 h. SmartFlare probes (3 μl) (Cy3 labeled mRNA, or scramble control detection probes, purchased from Millipore) pre-diluted into 50 μl of PBS were added to each well in triplicates. Cells were incubated overnight (˜16 h) at 37° C. and 5% CO₂ and analyzed with a fluorescent microscope, and digital pictures were taken with similar light exposure for expression of mRNA.

### Example 4: Detection of Protein Expression in Culture Media

The protein expressed by the mRNA construct was identified and quantitated by RP-HPLC using an analytical C18 column (250 mm×2.1 mm; Phenomenex). Protein detection used a dual wavelength detector. A gradient of was adjusted over time to allow analytical separation of protein peaks. In initial experiments, fractions were collected and submitted for Mass Spectrometry to determine the presence of the expected sequence. The secreted product and the product manufactured within the cells were compared using protein sequencing. To mitigate enzyme degradation of the sample, enzyme inhibitors were used in the media and concentrated media from multiple wells in order to detect the product on HPLC.

### Example 5: Determine Best Nanoparticle for Delivery

Sequence and Structure of Polymers: The biopolymer core facility at the University of Maryland synthesized HK polymers on a Rainin Voyager synthesizer (PTI). Linear, four-branched, and eight-branch HK peptides were investigated for their ability to carry mRNA. In the 4-branched HK peptides, the branches emanate from a 3-lysine core.

In Vitro mRNA Transfection: Several HK peptides were examined for their ability to carry a luciferase-expressing mRNA (Trilink Biotechnologies, Inc., CleanCap Firefly Luciferase mRNA) into MDA-MB-231 cells. In brief, 1×10⁵ cells were plated into a 24-well plate containing 500 μl of DMEM and 10% serum. After 24 h, when the cells were 60 to 80% confluent, the media in each well was changed to Opti-MEM. To prepare HK polyplexes, HK peptide (4 to 12 mg) was mixed in 50 ml of Opti-MEM, mRNA (1 mg) was briefly added into the mixture and maintained at room temperature for 30 min. This polyplex was then added dropwise to the cells. After 4 h, the Opti-MEM media was removed, and replaced with 1 ml of DMEM/10% serum. Twenty-four hours later, the cells were lysed, and the luciferase activity was measured.

Transfection with HK lipoplexes was done similarly as above with few exceptions. In brief, HK peptide was mixed initially with mRNA at various ratios and incubated for 30 minutes in Opti-MEM. This was followed by adding MC3 or DOTAP cationic liposome (1,2-dioleoyl-3-trimethylammonium-propane; 1 or 1.5 g; Roche) and an incubation for 30 min. The Opti-MEM mixture (1001) was then added to the cells.

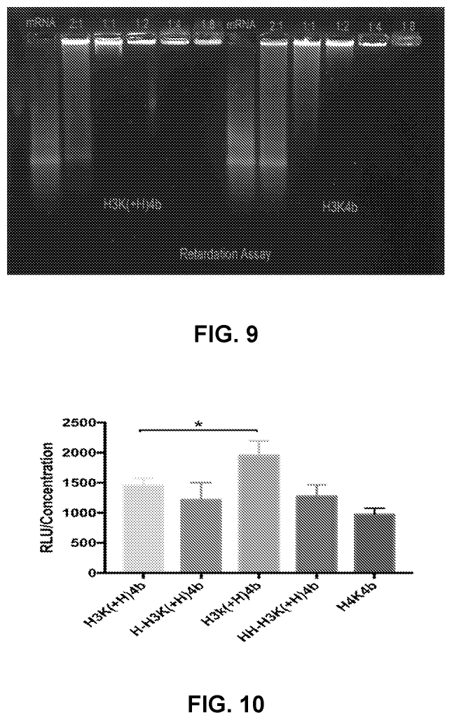

Gel Retardation Assay: Various amounts of HK peptides were mixed with 1 μg of mRNA and incubated for 30 min at room temperature. Specifically, the following HK/mRNA ratios (w/w) were prepared in water: 1/2; 1/1; 2/1, 4/1, 8/1. After 30 min, the HK polyplex was loaded onto the gel (1% agarose containing ethidium bromide), electrophoresis was then carried out at a constant voltage of 75 V for 60 minutes in TBE buffer. Images were acquired by the UV imager (ChemiDoc Touch, BIO-RAD, CA). See, for example, FIG. 9.

Heparin Displacement Assay: Complexation of HK and plasmids (4:1 wt/wt ratio) was assessed by a fluorescent assay in mQ water. Complexes were prepared as described previously, followed by the addition of diluted SG. For detection, the complexes (1/5 of volume), water (3/5) and SG (1/5) working dilution were incubated for 5 minutes and fluorescence was measured by a fluorimeter (λex=300 nm, λem=537 nm)(SynergyMx, BioTek). A control sample was prepared with the same amount of naked mRNA, water and SG. For the heparin displacement, instead of water, heparin salt (Sigma-Aldrich, St. Louis, MO, USA) solutions at different concentrations were used and the complexes were incubated at 37° C. for 30 min before addition of the SG dilution. Complex formation was also confirmed by gel electrophoresis.

Flow Cytometry: In brief, 1×10⁵ MDA-MB-231 cells were plated into 24-well plate containing 500 μl of DMEM and 10% serum. Transfection with HK polypeptides including H3K(+H)4b and H3K4b was conducted similarly as described above with cyanine 5-labeled mRNA (Trilink Biotechnologies, Inc., CleanCap Cyanine 5 Fluc mRNA). At the time of 30 minutes, 1, 2 and 4 hours after transfection, cells were digested and neutralized with 10% serum. A control sample without transfection was also collected. After centrifuging at 1000 rpm for 1 min, cells were resuspended with 250 μl PBS. For analysis, a typical forward- and side-scatter gate was set to exclude dead cells and aggregates. Events in each sample were collected using the Beckman Coulter Cytoflex (Beckman Coulter, CA, USA). The percentage of control sample was defined as 0%. The values of other samples were relative values and recorded as polycomplexes uptake percentages.

The results confirmed that both H3K4b and H3K(+H)4b are effective as carriers of mRNAs in vitro. H3K(+H)4b was shown as a markedly better as a carrier of mRNA compared to its close H3K4b analog (FIG. 8). The retardation assay showed the effect of polypeptides in different weight ratios of mRNA and polypeptide. The results indicated that the electrophoretic mobility of free mRNA was retarded by HK polypeptides. With 1:2 ratio and 1:4 ratio of H3K(+H)4b and H3K4b, the mRNA was completely trapped in the well, which indicate that H3K(+H)4b binds to mRNA more tightly than H3K4b (FIG. 9). All the HK peptides with an extra histidine in the second -FH HK (SEQ ID NO: 82) motif of the branches were effective carriers of mRNA (FIG. 9). Of these peptides, H3k(+H) was determined to be the optimal carrier of mRNA (H3k(+H)4b vs. H3K(+H)4b, P<0.05).

### Example 6: Synergistic Activity of MC3 or DOTAP with HK Carriers in mRNA Delivery

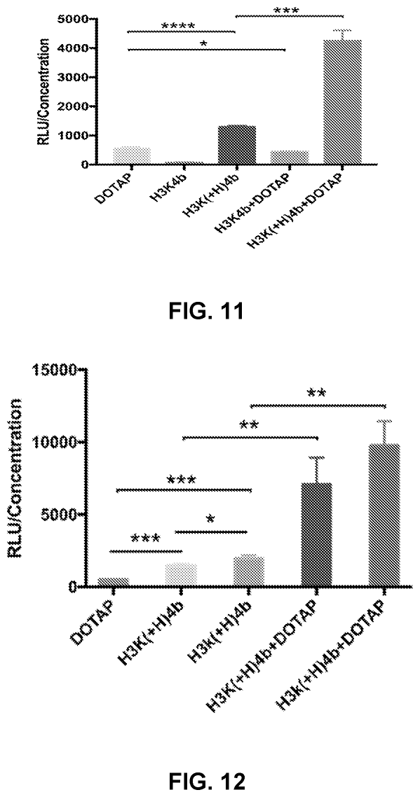

The combination of H3K(+H)4b and MC3/DOTAP liposomes was synergistic in its ability to carry mRNA into MDA-MB-231 cells (H3K(+H)4b/liposomes vs liposomes, P<0.0001). The combination was about 3-fold and 8-fold more effective as carriers of mRNA than the polymer alone and the liposome carrier, respectively. Notably, not all HK peptides demonstrated the synergistic activity with MC3/DOTAP liposomes. The combination of H3K4b and MC3/DOTAP carriers was less effective than the DOTAP liposomes as carriers of luciferase mRNA. Besides DOTAP and MC3, other cationic liposomes that may be used with HK peptides include Lipofectin (Invitrogen), Lipofectamine (Invitrogen), and DOSPER (FIG. 11).

The D-isomer of H3k (+H)4b, in which the L-lysines in the branches were replaced with D-lysines, was the most effective polymeric carrier (H3k(+H)4b vs. H3K(+H)4b, P<0.05). The D-isomer/liposome carrier of mRNA was nearly 4-fold and 10-fold more effective than the H3k(+H)4b alone and liposome carrier, respectively. Although the D-H3K(+H)k4b/liposome combination was modestly more effective than the L-H3K(+H)4b/liposome combination, this comparison was not statistically different (FIG. 12).

### Example 7: Spermine-Liposome Conjugates/mRNA Nanoparticle Preparation

A spermine-liposome conjugates (SLiC) delivery system (FIG. 13) was also developed. Regular methods were tried at first to prepare liposomes with newly synthesized SLiC molecules, such as thin film method, solvent injection etc. without much success. As described herein, lipids dissolved in ethanol are in a so-called metastable state in which liposomes are not very stable and tend to aggregate. Un-loaded or pre-formed liposomes were then prepared using a modified Norbert Maurer's method. It was found that stable liposome solution can be made by simply diluting ethanol to the final concentration of 12.5% (v/v). Liposomes were prepared by addition of lipids (cationic SLiC/cholesterol, 50:50, mol %) dissolved in ethanol to sterile dd-H₂O. The ethanolic lipid solution was added slowly under rapid mixing.

Slow addition of ethanol and rapid mixing proved successful in making SLiC liposomes, as the process allows formation of small and more homogeneous liposomes. Unlike conventional methods, in which mRNAs are loaded during the process of liposome formulation and ethanol or other solvent is removed at end of manufacturing, these SLiC liposomes were formulated with ethanol still remaining in the solution so that liposomes were thought to be still in a metastable state. When the mRNA solution was mixed/loaded with liposome solution cationic groups, lipids interact with anionic mRNA and condense to form a core. The SLiC liposomes' metastable state helped or facilitated liposome structure transformation to entrap mRNA more effectively. Because of the entrapment of mRNA, SLiC liposomes became more compact and homogeneous.

### Example 8: Development and Characterization of Nanoparticles for In Vivo Delivery of mRNA

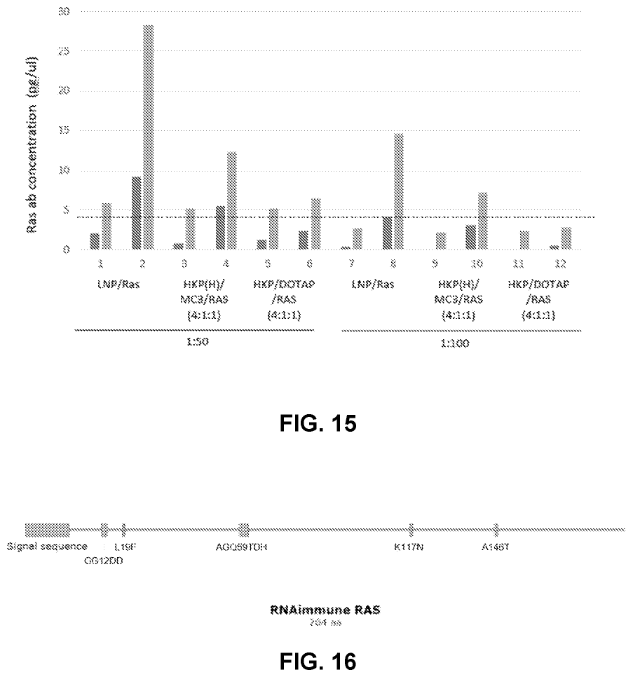

Developing an mRNA-based vaccine includes a successful delivery of the mRNA into the cells. As an example of vaccine delivery methods, mRNA expressed in vitro with a linearized plasmid based construct with 5′ and 3′ UTRs, including a poly-A tail, was collected and quantified. In one example, mRNA, HKP+H polymer and MC3 mixture was prepared with weight ratio of 1:2:4. In another example, mRNA, HKP+H polymer and PLA mixture was prepared with weight ratio of 1:2:4. In yet another example, lipid nanoparticles was prepared using the mixture of MC3, DSPC, CHOL and DSPE-PEG2000 with molar ratio 50:10:38.5:1.5. The LNP was then mixed with mRNA with a weight ratio of 4:1. All formulations were tested for particle size, mRNA encapsulation, and endotoxin prior to injection into animals. A single dose of 50-20011.1 solution was injected into mice. Delivery methods include intratumoral, intravenous, intramuscular, intradermal, and subcutaneous injection. In a specific example, the mRNA constructs expressing epitopes of ras neoantigens were formulated with different HK peptides and injected into mice (30 μg/dose) RAS antibody titer was assessed by ELISA (FIG. 15).

The pan-RAS antigen of SEQ ID NO:70 contains all identified RAS amino acid alternations. The full length mutated ras mRNA can be packaged directly by adding the delivery nanoparticle to the full length mutated ras mRNA, without the need for linkers or construction of a minigene as described above.

### Example 9: Design mRNA Targeting Kras Mutations

As described herein, the vaccine comprises, or consists essentially of, or yet further consists of a synthetic mRNA containing a whole or part of several protein-encoding open reading frames (ORFs). Particularly a ras neoantigen further comprising the SARS-cov2 signal sequence.

### Example 10: Cancer Vaccine: Pan-Ras Neoantigen

KRAS mutation (downstream of the EGFR protein) results in constitutive activation of the RAS-RAF-ERK pathway and is hypothesized to cause resistance to anti-EGFR therapy. Majority of the mutations are at one of three mutational hotspots: G12, G13 and Q61 (COSMIC v92). Mutations are also localized to other codons, such as 19, 117 and 146 have been shown to have phenotypes similar to the hotspot mutations, such as those disclosed in Table 3 or cancer.sanger.ac.uk/cosmic/gene/analysis?ln=KRAS, last accessed on Sep.s 21, 2021. In some embodiments, the selected mutations comprise, or consist essentially of, or yet further consist of those with the highest frequency at the mutation spot.

TABLE 3
Frequency of KRAS mutations at selected amino acids.

| Position | Mutation | Mutation | |
|---|---|---|---|
| (AA) | (CDS) | (Amino Acid) | Count |
| 12 | c.35G > A | p.G12D | 15848 |
| 13 | c.38G > A | p.G13D | 5986 |
| 19 | c.53G > C | p.L19F | 21 |
| 19 | c.53G > T | p.L29F | 24 |
| 59 | c.175G > A | p.A59T | 38 |
| 60 | c.179G > A | p.G60D | 12 |
| 61 | c.183A > C | p.Q61H | 324 |
| 61 | c.183A > T | p.Q61H | 143 |
| 117 | c.351A > C | p.K117N | 28 |
| 117 | c.351A > T | p.K117N | 47 |
| 146 | c.436G > A | p.A146T | 296 |

As shown in FIG. 16, a 615 bp ras gene encodes a 204 aa RAS protein, comprising 8 mutations covering all mutational hotspots. The signal sequence of a SPIKE protein from SARS-COV-2 was used. The designed RNAs were ordered from two vendors, Trillink and Codex.

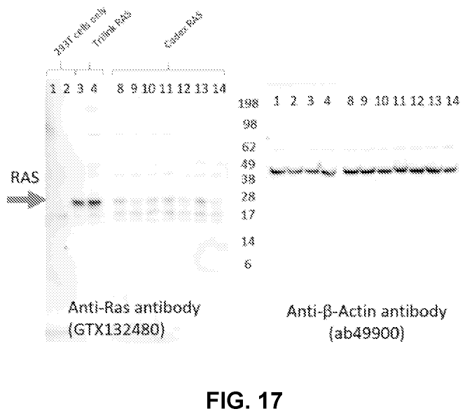

RAS expression was confirmed using western blot analysis. As shown in FIG. 17, much stronger RAS band was detected from Trilink supplied RNA, while the Codex RNA was shown as functional but expressed at a much lower level. Loading controls showed similar protein contents. Also, the background noise signal was very low.

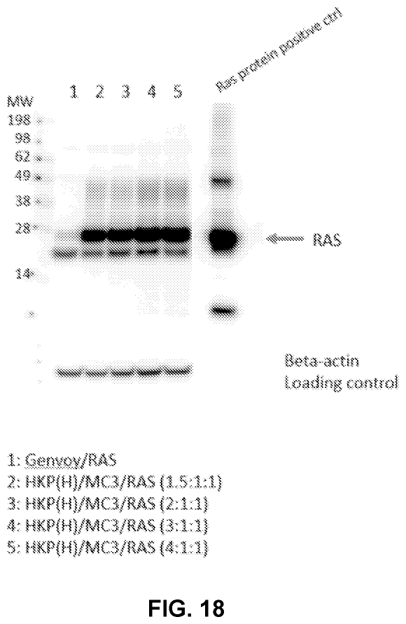

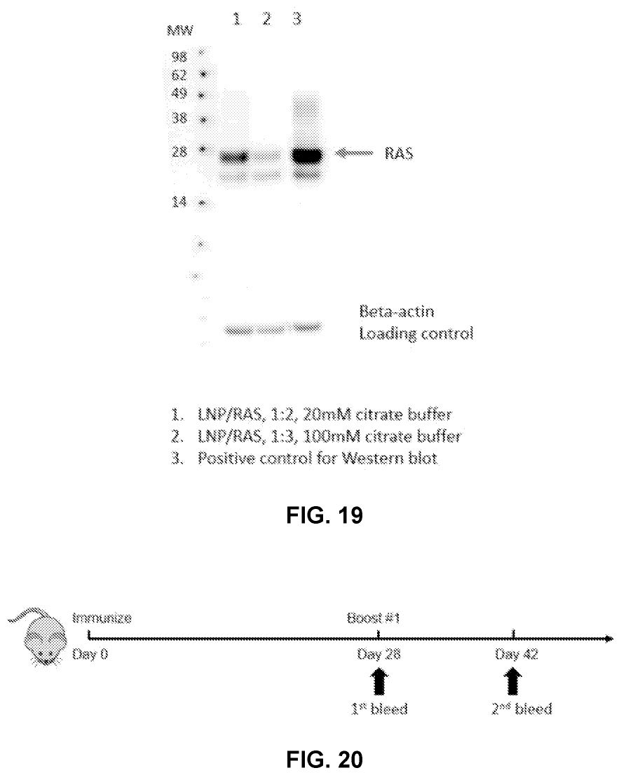

In vitro RAS expression was further evaluated using LNP or HKP(H) formulation. A representative result is shown in FIG. 18 and FIG. 19. A significant expression of RAS protein after transfection with HKP(H) formulated ras mRNA or LNP formulated ras mRNA was observed.

### Example 11: Animal Study: A Mouse In Vivo Study—Treatment Approach

As illustrated in FIG. 20, Balb/c mice were immunized intramuscularly (i.m.) with ras mRNA vaccine formulated with various nanoparticle, such as LNP (1:3), HKP(H)/MC3 (4:1:1) or HKP/DOTAP (4:1:1). 2 mice were tested for each group. Sera were collected on Day 28 before the first boost and 14 days after first boost (i.e., on Day 42). ELISA was performed on sera collected on Day 28 and Day 42 to detect anti-RAS antibodies induced as well as to identify the antibodies' IgG isotype. Mice were sacrificed, spleens were removed, then RNA was extracted for qRT-PCR in order to measure gene expression of Th1 and Th2 related genes and other genes.

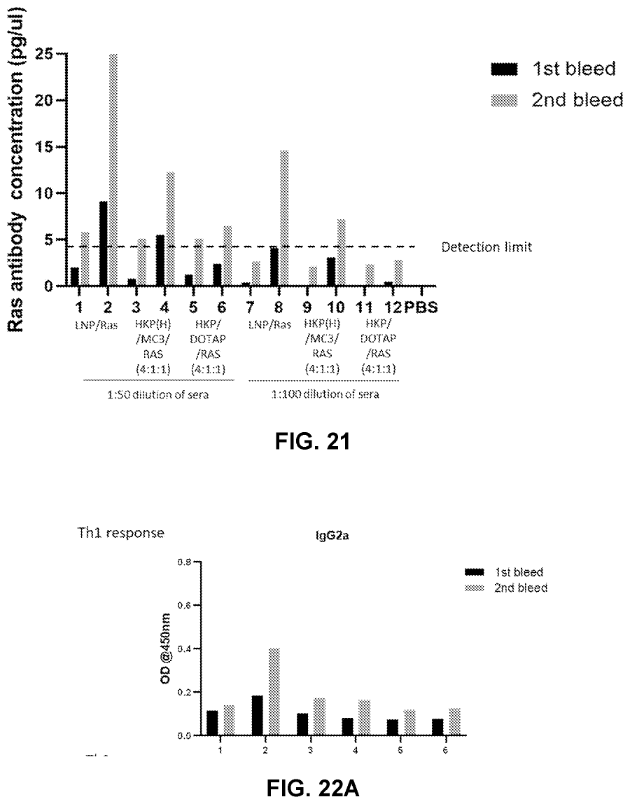

The obtained ELISA result detecting anti-RAS antibodies in the collected sera is shown in FIG. 21. It shows that after the boost, anti-RAS antibodies were readily detected in mouse sera.

Th1 cytokines promote the development of an anti-tumor cell-mediated immune response. Therefore, for an ideal KRAS cancer vaccine, Th1 response is critical. See, for example, Lin, et al. (2017). International Journal of Head and Neck Science, Vol 1. No. 2, Jun. 1, 2017, pages 105-113. Naïve T cells become Th1 cells or Th2 cells, following the stimulation by different factors. In Th1 immunity, cells produce pro-inflammatory cytokines, such as interleukin-2 (IL-2), interferon-gamma (IFN-γ), tumor necrosis factor-beta (TNF-β). In Th2 immunity, cells produce anti-inflammatory cytokines, such as IL-4, IL-5, IL-6, IL-10 and IL-13. In normal circumstances Th1 immunity and Th2 immunity approach a balance. But, the presence of tumor cells disrupts this balance. This occurring increased Th2 immunity and decreased Th1 immunity, because of down-regulation of adaptive immunity. This eventually leads to tumor progression. However, if Th1 immunity becomes predominant, this stimulation of immunity can lead to tumor regression.

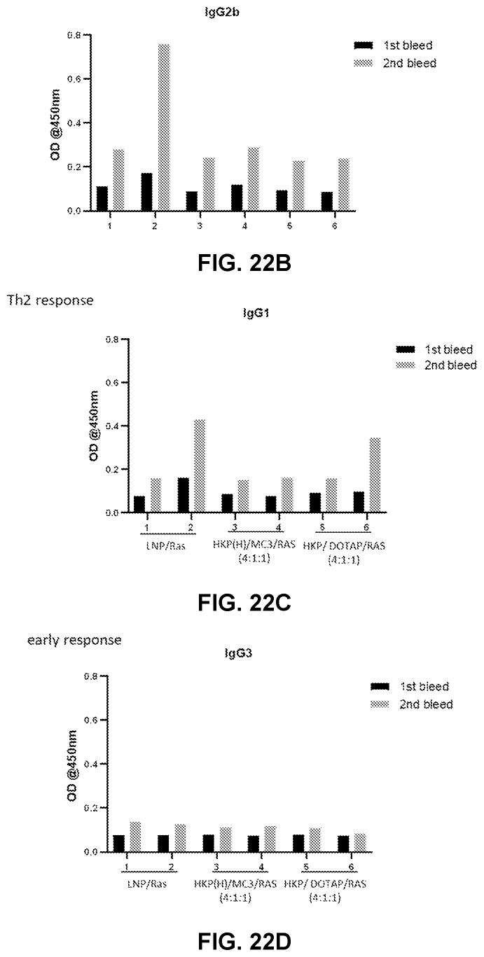

IgG isotype can predict the T helper phenotype involved in initiating the immune response in an animal model: IgG2a and IgG2b are correlated with Th1 response; gG1 is correlated with Th2 response; and IgG3 normally appears early in response. Accordingly, IgG isotype of the anti-RAS antibodies induces in mice was evaluated and the result is shown in FIG. 22. The dominant IgG isotype in mice immunized with Ras vaccine was shown as IgG2b.

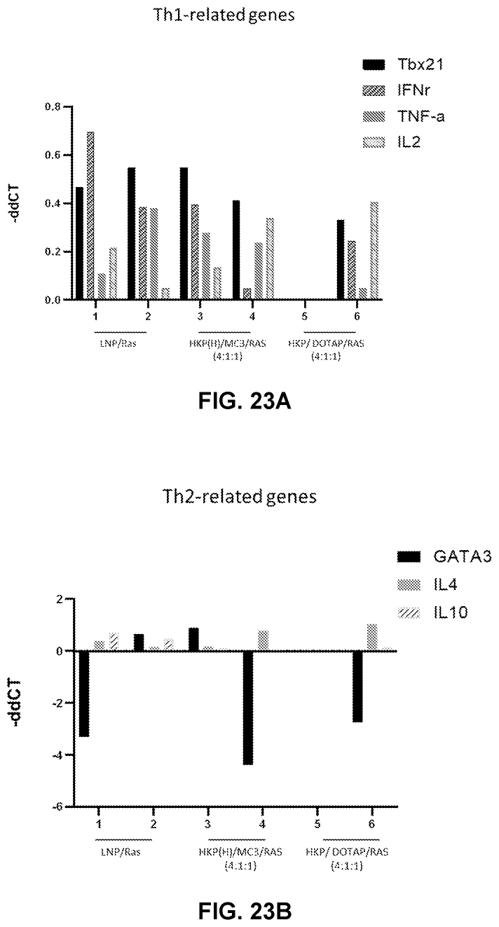

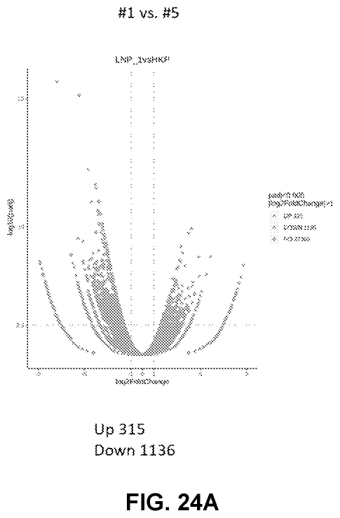

Further, gene expression of Th1 and Th2 related genes was evaluated. Briefly, RNAs were isolated from spleen. Th1 related genes, such as Tbet (Tbx21), IFN-γ, IL-2, and TNF, as well as Th2 related genes, such as GATA3, IL-4, and IL-10, were evaluated using qRT-PCR and NGS. The RT-PCR result is shown in FIG. 23. No actual negative control was used while mouse #5 was provided as a relative control.

The obtained RNAs were also evaluated for transcriptomics profiling using next-generation sequencing (NGS). Briefly, RNAs from spleen were isolated from 6 mice, and analyzed using NGS. After quality control, NGS was performed using RNAs from mice #1, #2, #3, and #5. Such mouse numbering is also used in FIGS. 21-23. Additionally, based on the ELISA result, #5 mouse was used as relative negative control.

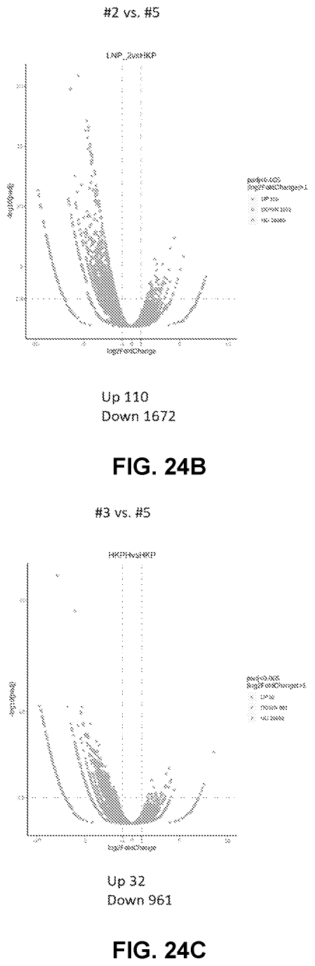

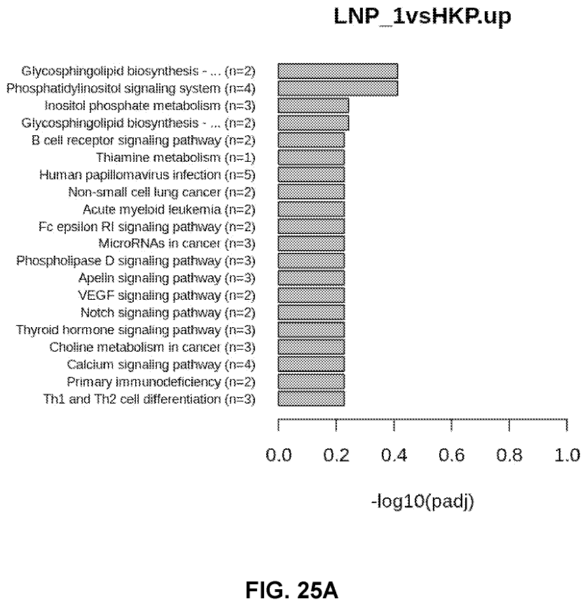

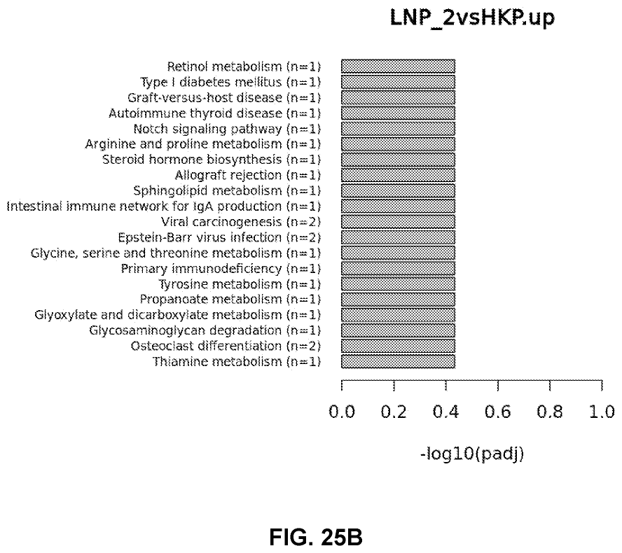

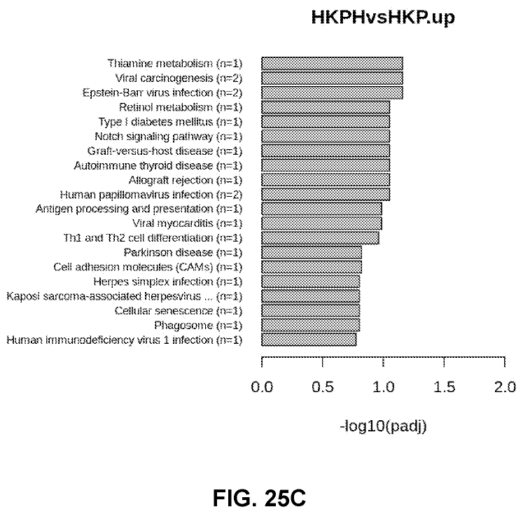

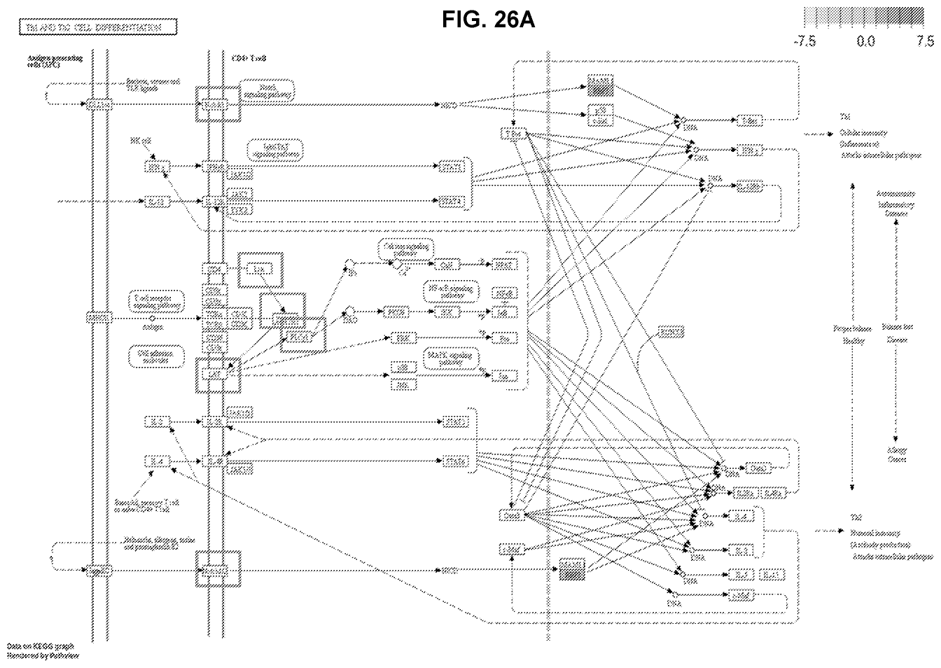

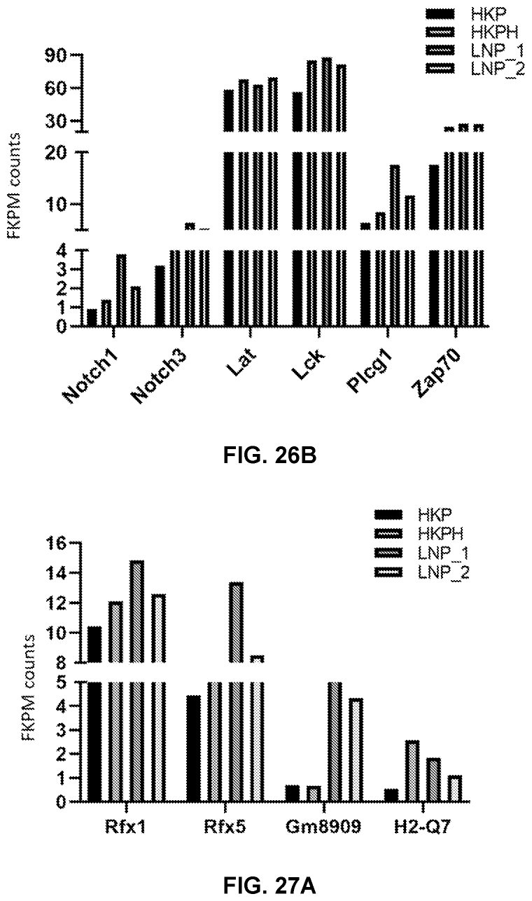

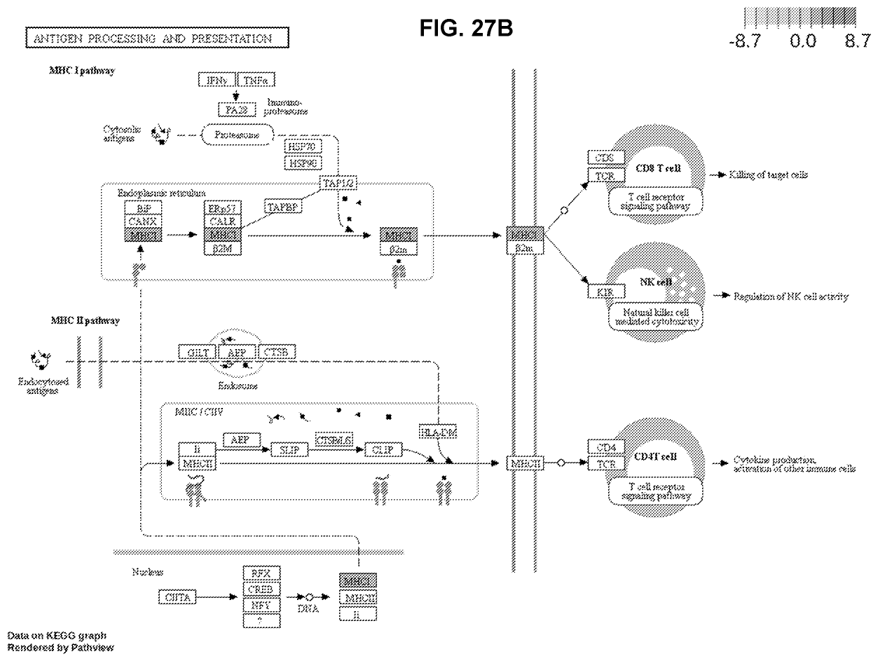

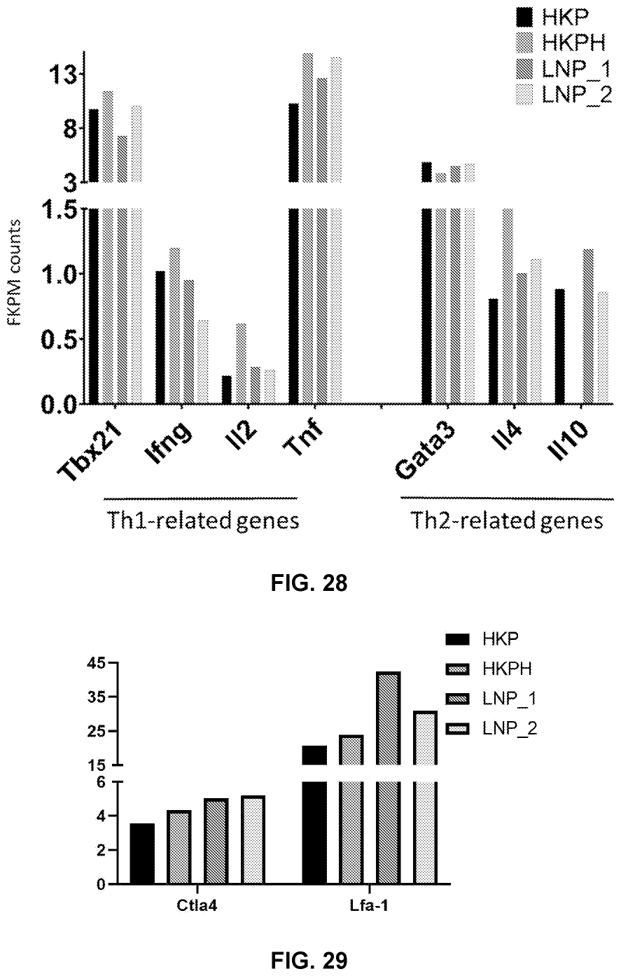

The NGS analysis results are then disclosed herein. Briefly, the differential expressed genes are plotted in FIG. 24, while the top 20 KEGG pathways, including the Th1 and Th2 cell differentiation pathway, are identified in FIG. 25. Further, expression levels of six genes involved in the Th1 and Th2 cell differentiation pathway, i.e., Notch 1, Notch 3, Lat, Lck, Plcg1 and Zap70, were plotted as FKPM counts in FIG. 26B. The Th1 and Th2 related genes investigated as shown in FIG. 23 using qRT-PCR were also analyzed using NGS, and the result is plotted in FIG. 28. The results show that: the master transcription factor of Th1, Tbx21, was slightly increased in two mice, while the master transcription factor of Th2, GATA3, was slightly decreased in three mice when compared with mouse #5; Th1 related genes, IL-2 and TNF were increased; and Th2 related genes, IL4 and IL10 were observed with either no change or slightly increase, which is consistent with qRT-PCR results as shown in FIG. 23. It indicates that the tested formulated mRNAs stimulate anti-cancer Th1 immune responses.

Additionally evaluated were expression levels of four genes involved in antigen processing and presentation pathway, including Rfx1, Rfx5, Gm89096 and H2-Q7.

Several markers for CD8+ T cell activation have been identified, such as LFA-1 and CTLA-4. See, for example, Slifka M K and Whitton J L. J Immunol. 2000 Jan. 1; 164(1):208-16, which is incorporated herein by reference in its entirety. Accordingly, these two phenotypic marks for activated CD8+ cells were also evaluated and the result is shown in FIG. 29. Increased expression was observed indicating activation of CD8+ cells was improved by the tested formulated mRNAs.

The anti-tumor effects of the formulated mRNAs were also tested in vivo. Briefly, Balb/c mice were immunized with 100 μl of the formulated mRNA prepared as described herein per mice on Day 0 and another 100 μl per mice on Day 10. CT26 cells were inoculated to the hind leg via subcutaneous injection. The animal were euthanized on Day 14.

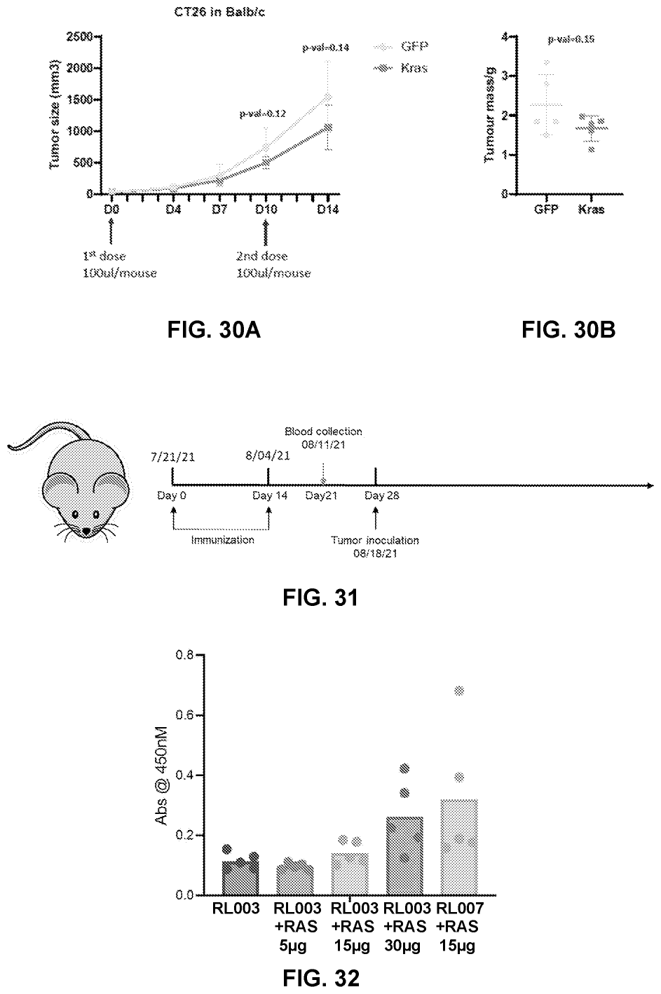

The tumor sizes were measured daily and the result is plotted in FIG. 30A. After sacrificing the mice, the tumor mass was dissected and weighted, and the result is plotted in FIG. 30B. Smaller and lighter tumors were observed in mice treated with the formulated mRNA, indicating such formulated mRNA can be used as a promising anti-cancer drug.

### Example 12: A Preventive Approach

The formulated mRNA were produced and tested in vivo with dose titration as indicated in Table 4. RL003 indicates the mRNA is formulated using MC3 while RL007 indicated the mRNA is formulated using SM102. The MC3 formulation is served as a negative control while the SM102 formulated ras mRNA is considered as a positive control.

TABLE 4
Animal cohorts and major experimental procedures.

| | | mRNA | μg/dose | animals | Day 0 | Day 14 | Day 21 | Day 28 |
|---|---|---|---|---|---|---|---|---|
| 1 | RL003 | 0 | 0 | 5 | 1st | 2nd | 1 st | Tumor |
| | | | | | injection | injection | bleeding | inoculation, |
| | | | | | | | | 2 × 10⁵ cell |
| | | | | | | | | injection |
| 2 | RL003 | RAS | 5 | 5 | 1st | 2nd | 1st | Tumor |
| | | | | | injection | injection | bleeding | inoculation, |
| | | | | | | | | 2 × 10⁵ cell |
| | | | | | | | | injection |
| 3 | RL003 | RAS | 15 | 5 | 1st | 2nd | 1st | Tumor |
| | | | | | injection | injection | bleeding | inoculation, |
| | | | | | | | | 2 × 10⁵ cell |
| | | | | | | | | injection |
| 4 | RL003 | RAS | 30 | 5 | 1^{st} | 2^{nd} | 1st | Tumor |
| | | | | | injection | injection | bleeding | inoculation, |
| | | | | | | | | 2 × 10⁵ cell |
| | | | | | | | | injection |
| 5 | RL007 | RAS | 15 | 5 | 1st | 2nd | 1st | Tumor |
| | | | | | injection | injection | bleeding | inoculation, |
| | | | | | | | | 2 × 10⁵ cell |
| | | | | | | | | injection |

As illustrated in FIG. 31, mice aged 6-7 weeks were immunized with the RAS vaccine as disclosed herein or controls on Day 0 and Day 14. 5 mice were tested in each group, and a total of 5 groups as shown in Table 4 were investigated. Blood was collected on Day 21 to measure the anti-RAS antibody production. On Day 28, mice are challenged intramuscularly (i.m.) with 2×10⁵ CT26 cells. Tumor growth is monitored and survival curve is generated. T-cell mediated immune response is assessed and a transcriptomic analysis is performed at the end of study as described herein. An ELISA result detecting anti-RAS antibodies in sera after the first vaccine injection is shown in FIG. 32. A dosage dependent effect was observed.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. An isolated ribonucleic acid (RNA) comprising an open reading frame (ORF) encoding a ras derived peptide, wherein the encoded ras derived peptide comprises any one or more of the following mutations:
a phenylalanine (F) aligned to the 19^{th} amino acid residue of SEQ ID NO: 70;
a threonine (T) aligned to the 59^{th} amino acid residue of SEQ ID NO: 70;
an aspartic acid (D) aligned to the 60^{th} amino acid residue of SEQ ID NO: 70;
an asparagine (N) aligned to the 117^{th} amino acid residue of SEQ ID NO: 70; or
a T aligned to the 146^{th} amino acid residue of SEQ ID NO: 70,
and wherein the RNA is encapsulated in a nanoparticle.

2. The isolated RNA of claim 1, wherein the encoded ras derived peptide further comprises any one or more of the following mutations:
a D aligned to the 12^{th} amino acid residue of SEQ ID NO: 70,
a D aligned to the 13^{th} amino acid residue of SEQ ID NO: 70; or
a histidine (H) aligned to the 61^{th} amino acid residue of SEQ ID NO: 70.

3. The isolated RNA of claim 2, wherein the encoded ras derived peptide comprises the following mutations:
D aligned to the 12^{th} amino acid residue of SEQ ID NO: 70,
D aligned to the 13^{th} amino acid residue of SEQ ID NO: 70;
F aligned to the 19^{th} amino acid residue of SEQ ID NO: 70;
T aligned to the 59^{th} amino acid residue of SEQ ID NO: 70;
D aligned to the 60^{th} amino acid residue of SEQ ID NO: 70;
H aligned to the 61^{th} amino acid residue of SEQ ID NO: 70;
N aligned to the 117^{th} amino acid residue of SEQ ID NO: 70; or
T aligned to the 146^{th} amino acid residue of SEQ ID NO: 70.

4. The RNA of claim 3, wherein the ras derived peptide comprises the polypeptide as set forth in SEQ ID NO: 70, or an equivalent thereof retaining the following mutations:
D aligned to the 12^{th} amino acid residue of SEQ ID NO: 70,
D aligned to the 13^{th} amino acid residue of SEQ ID NO: 70;
F aligned to the 19^{th} amino acid residue of SEQ ID NO: 70;
T aligned to the 59^{th} amino acid residue of SEQ ID NO: 70;
D aligned to the 60^{th} amino acid residue of SEQ ID NO: 70;
H aligned to the 61^{th} amino acid residue of SEQ ID NO: 70;
N aligned to the 117^{th} amino acid residue of SEQ ID NO: 70; or
T aligned to the 146^{th} amino acid residue of SEQ ID NO: 70.

5. A ribonucleic acid (RNA) comprising an open reading frame (ORF) encoding one or more ras derived peptides, wherein each of the one or more ras derived peptides consists of between 23 and 29 amino acid residues, and wherein the encoded peptides are selected from the group as set forth in SEQ ID NOs:1-69, or an equivalent of each thereof.

6. The RNA of claim 5, wherein the ras derived peptides are selected from the group as set forth in SEQ ID NOs:1-31, or an equivalent of each thereof.

7. The RNA of claim 5, wherein the ras derived peptides are selected from the group as set forth in SEQ ID NOs:32-52, or an equivalent of each thereof.

8. The RNA of claim 5, wherein the ras derived peptides are selected from the group as set forth in SEQ ID NOs: 53-69 or an equivalent of each thereof.

9. The RNA of claim 5, wherein the ras derived peptides are selected from the group as set forth in SEQ ID NOs: 19-31, 50-52 or 69.

10. The RNA of claim 9, wherein the ORF encodes the polypeptide as set forth in SEQ ID NO: 70, or an equivalent thereof retaining the following mutations:
D aligned to the 12^{th} amino acid residue of SEQ ID NO: 70,
D aligned to the 13^{th} amino acid residue of SEQ ID NO: 70;
F aligned to the 19^{th} amino acid residue of SEQ ID NO: 70;
T aligned to the 59^{th} amino acid residue of SEQ ID NO: 70;
D aligned to the 60^{th} amino acid residue of SEQ ID NO: 70;
H aligned to the 61^{th} amino acid residue of SEQ ID NO: 70;
N aligned to the 117^{th} amino acid residue of SEQ ID NO: 70; or
T aligned to the 146^{th} amino acid residue of SEQ ID NO: 70.

11. The RNA of claim 4, wherein the ORF comprises the polynucleotide as set forth in AUGUUUGUUUUUCUUGUUUUAUUGCCACUAGUCUCUAGUCAGUGUAUGACUGAA UAUAAACUUGUGGUAGUUGGAGCUGAUGACGUAGGCAAGAGUGCCUUUACGAUA CAGCUAAUUCAGAAUCAUUUUGUGGACGAAUAUGAUCCAACAAUAGAGGAUUCC UACAGGAAGCAAGUAGUAAUUGAUGGAGAAACCUGUCUCUUGGAUAUUCUCGAC ACAACAGAUCACGAGGAGUACAGUGCAAUGAGGGACCAGUACAUGAGGACUGGG GAGGGCUUUCUUUGUGUAUUUGCCAUAAAUAAUACUAAAUCAUUUGAAGAUAUU CACCAUUAUAGAGAACAAAUUAAAAGAGUUAAGGACUCUGAAGAUGUACCUAUG GUCCUAGUAGGAAAUAAUUGUGAUUUGCCUUCUAGAACAGUAGACACAAAACAG GCUCAGGACUUAGCAAGAAGUUAUGGAAUUCCUUUUAUUGAAACAUCAACAAAG ACAAGACAGAGAGUGGAGGAUGCUUUUUAUACAUUGGUGAGAGAGAUCCGACAA UACAGAUUGAAAAAAAUCAGCAAAGAAGAAAAGACUCCUGGCUGUGUGAAAAUU AAAAAAUGCAUUAUAAUGUAA (SEQ ID NO: 88) or nucleotide (nt) 1 to nt 612 of SEQ ID NO: 88, or an equivalent thereof encoding the same ras derived peptide.

12. The RNA of claim 5, wherein the ORF encodes a polypeptide comprising two or more ras derived peptides and a peptide linker between any two adjacent ras derived peptides.

13. The RNA of claim 1, wherein the encoded ras derived peptide or peptides comprise a wildtype residue aligned to the 12^{th} amino acid residue of SEQ ID NO: 70, or a wildtype residue aligned to the 13^{th} amino acid residue of SEQ ID NO: 70, or both.

14. The RNA of claim 1, wherein the ORF further encodes a signal peptide, optionally wherein the single peptide comprises MFVFLVLLPLVSSQC (SEQ ID NO: 87).

15. The RNA of claim 1, further comprising a 3′-UTR and a 5′-UTR.

16. The RNA of claim 15, wherein the 5′-UTR comprises an m7G cap structure and a start codon, optionally wherein the 5′-UTR comprises AGGacaUUUgcUUcUgacacaacUgUgUUcacUagcaaccUcaaacagacaCCGCCACC (SEQ ID NO: 89) or an equivalent thereof.

17. The RNA of claim 15, wherein the 3′-UTR comprises a stop codon and a polyA tail, optionally wherein the 3′-UTR comprises GCUCGCUUUCUUGCUGUCCAAUUUCUAUUAAAGGUUCCUUUGUUCCCUAAGUCCA ACUACUAAACUGGGGGAUAUUAUGAAGGGCCUUGAGCAUCUGGAUUCUGCCUAA UAAAAAACAUUUAUUUUCAUUGCCAAUAGGCCGAAAUCGGCAAGCGCGAUCGC (SEQ ID NO: 90) or an equivalent thereof.

18. The RNA of claim 1, prepared by transcribing a polynucleotide encoding the RNA in an in vitro transcription (IVT) system.

19. The RNA of claim 1, prepared by transcribing a plasmid DNA (pDNA) vector, optionally a pUC57 plasmid, encoding the RNA, optionally wherein the plasmid comprises SEQ ID NO: 91 or an equivalent thereof.

20. The RNA of claim 1, wherein the GC content of the full-length RNA is about 35% to about 70% of the total RNA content.

21. The RNA of claim 1, wherein the RNA is chemically modified, optionally wherein the chemical modification comprising one or both of the incorporation of an N1-methyl-pseudouridine residue or a pseudouridine residue, further optionally wherein at least about 50% to about 100% of the uridine residues in the RNA are N1-methyl pseudouridine or pseudouridine.

22. A polynucleotide encoding the RNA of claim 1, or a polynucleotide complementary thereto, or both.

23. (canceled)

24. A vector comprising the polynucleotide of claim 22.

25.-31. (canceled)

32. A cell comprising one or more of: the RNA of claim 1.

33.-34. (canceled)

35. A composition comprising a carrier, optionally a pharmaceutically acceptable carrier, and the RNA of claim 1.

36. A method of producing an RNA, comprising culturing the cell of claim 32 under conditions suitable for transcribing a DNA encoding the RNA to the RNA.

37. A method of producing an RNA, comprising contacting the polynucleotide of claim 22 an RNA polymerase, adenosine triphosphate (ATP), cytidine triphosphate (CTP), guanosine-5′-triphosphate (GTP), and uridine triphosphate (UTP) or a chemically modified UTP under conditions suitable for transcribing the polynucleotide or the vector to the RNA.

38. (canceled)

39. An RNA produced by the method of claim 36.

40. An immunogenic composition comprising an effective amount of the RNA of claim 1 formulated in a pharmaceutically acceptable carrier, optionally a polymeric nanoparticle or a liposomal nanoparticle or both, or further optionally.

41. The composition according to claim 40, wherein the pharmaceutically acceptable carrier comprises a polymeric nanoparticle or a liposomal nanoparticle or both optionally wherein the liposomal nanoparticle is selected from a Histidine-Lysine co-polymer (HKP) or H3K(+H)4b or H3k(+H)4b or both or wherein the polymeric nanoparticle comprises a lipid, a lipid nanoparticle (LNP), a cationic lipid, and optionally wherein the cationic lipid is ionizable.

42.-52. (canceled)

53. The composition of claim 41, wherein the LNP comprises a lipid, optionally a cationic lipid, optionally wherein the cationic lipid is ionizable, and optionally wherein the LNP comprises one or more of: 9-Heptadecanyl 8-{(2-hydroxyethyl)[6-oxo-6-(undecyloxy)hexyl]amino}octanoate (SM-102), 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), or an equivalent of each thereof.

54.-57. (canceled)

58. The composition of claim 41, wherein the LNP comprises SM-102, DSPC, cholesterol and PEG2000-DMG.

59. The composition of claim 58, wherein the mass ratio of the SM-102, DSPC, cholesterol and PEG200-DMG is about 1:1:1:1 and/or wherein the molar ratio of the SM-102, DSPC, cholesterol and PEG2000-DMG is about 50:10:38.5:1.5.

60. A method of producing a composition, comprising contacting the RNA of claim 1 with an HKP or a lipid, thereby preparing the composition.

61.-69. (canceled)

70. A method of treating a subject having a cancer, the method comprising administering to said subject a pharmaceutically effective amount of the polynucleotide of claim 22.

71.-74. (canceled)

75. A kit comprising instructions for use and the RNA of claim 1.
